(19) United States
(12) Patent Application Publication
(10) Pub. No.: US 2009/0092983 A1
(43) Pub. Date: Apr. 9, 2009

(54) IDENTIFICATION OF AN ERBB2 GENE EXPRESSION SIGNATURE IN BREAST CANCERS

(76) Inventors: Daniel Birnbaum, Marseille (FR); Francois Bertucci, Marseille (FR); Jocelyne Jacquemier, Marseille (FR); Stephane Debono, Marseille (FR); Nathalie Borie, Marseille (FR); Christophe Ginestier, Marseille (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 12/212,282

(22) Filed: Sep. 17, 2008

Related U.S. Application Data
(62) Division of application No. 10/928,465, filed on Aug. 27, 2004, now abandoned.
(60) Provisional application No. 60/498,497, filed on Aug. 28, 2003.

(51) Int. Cl. C12Q 1/68

(52) U.S. Cl. 435/6

(57) ABSTRACT

A method for analyzing differential gene expression associated with breast tumor is based on the analysis of the over-expression or under-expression of polynucleotide sequences in a biological sample. The analysis includes the detection of the over-expression of at least one polynucleotide sequence(s), subsequence(s) or complement(s) thereof selected from predefined polynucleotide sequence sets.

### RELATED APPLICATIONS

This is a divisional of U.S. Ser. No. 10/928,465, filed Sep. 27, 2004, which claims the benefit of U.S. Ser. No. 60/498,497, filed on Aug. 28, 2003, the entire disclosure of which is herein incorporated by reference.

### TECHNICAL FIELD

This disclosure relates to polynucleotide analysis and, in particular, to polynucleotide expression profiling of breast tumors and cancers using libraries or arrays of polynucleotides.

### BACKGROUND

The ERBB2 oncogene, also called HER2 or NEU, is located in band q12 of chromosome 17. It codes for a 185-kDa transmembrane tyrosine kinase related to members of the ERBB family, which also includes epidermal growth factor receptor. ERBB2 is amplified and over-expressed in 15-30% of breast cancers (1). Although its exact role in mammary oncogenesis remains unclear (2, 3, for reviews), the receptor is a clinically relevant target for the treatment of breast cancer for two reasons. First, ERBB2 gene amplification and over-expression of ERRB2 gene products have been associated in many studies with prognosis or response to anticancer therapies (4, 5, for reviews). Second, therapy based on a humanized monoclonal antibody (trastuzumab/Herceptin™) aimed at reducing the aberrant expression of the receptor has shown benefits in metastatic breast cancer patients (6-8, for reviews). However, modifications of chemotherapy and hormonal therapy strategies based on ERBB2 status remain controversial. Furthermore, the clinical efficacy of trastuzumab is unexpectedly variable, implying that additional and/or alternate methods to accurately identify appropriate patients for treatment with ERBB2 antagonists may be warranted.

Currently, ERBB2 status is primarily determined by two different methods: fluorescence in situ hybridization (FISH), which reveals gene amplification, and immunohistochemistry (IHC), which detects the over-expressed ERBB2 protein (9-12, for recent reviews). FISH is a good method for ERBB2 testing, but is technically more difficult to implement than IHC. IHC is easier to perform, but is difficult to standardize (13). IHC is currently the only FDA-approved test for selection of patients for treatment with trastuzumab. The American Society for Clinical Oncology and National Comprehensive Cancer Network guidelines recommend the use of either FISH (PathVysion™) or the HercepTest™, which is a specific IHC test made by the Dako Corporation.

This Herpceptin™ method includes a calibrated internal control to semi-quantitatively assess positive staining on a scale ranging from 0 (absence of ERBB2 protein over-expression) to 3+ (maximum of ERBB2 over-expression). Results are scored by a pathologist; interpretation is relatively straightforward in ERBB2-negative individuals (0-1+) and in patients who strongly over-express the protein (3+). Accurate scoring is however problematic for the intermediate level 2+. For cases scoring 2+(10-15% of all breast cancers), the concordance with FISH is, at best, 25%. Importantly, a proportion of 2+ cases are bona fide ERBB2-over-expressing tumors to which Herceptin treatment should be applied.

Thus, universal, accurate, and standardized determination of ERBB2 status has not yet been achieved. The reliability of this determination will greatly influence the selection of the relevant cases and thus the clinical efficacy of Herceptin treatment. Moreover, the establishment of specific methods for patient selection for ERBB2 antagonists may serve as a paradigm for guiding clinical use of the new targeted approaches expected in the near future. It is thus important to further document the methods and parameters useful to assess ERBB2 status.

Moreover, preliminary reports suggest that clinical outcome may vary between patients with the same ERBB2 status and treatment, implying that other factors, in addition to ERBB2, may play a role in determining the level of sensitivity to trastuzumab. Additionally, it may be necessary to associate other targeted therapies to anti-ERBB2 treatment, and identification of complementary or secondary targets may thus prove useful to guide selection of appropriate combination therapy. These secondary targets may contribute to activation of pathways associated with response to ERBB2 hyperactivity. Although the common pathways such as the RAS/MAPK pathway and other induced genes have been reported (14), ERBB2-associated signaling cascades have yet to be elucidated. Thus, accurate measurement of ERBB2 status as well as identification of associated molecular alterations are now intensively required.

The effect of surgery on proliferation of breast carcinomas, in particular those over-expressing HER2 oncoprotein, has been recently assessed (67). It has been found that residual breast carcinomas that had been surgically removed within 48 days after first surgery showed a significant increase in proliferation if they were ERBB2-positive. Treatment of ERBB2-positive tumour cells with trastuzumab before adding a growth stimulus abolished drainage-fluid-induced proliferation. This suggests that ERBB2 over-expression by breast carcinoma cells has a role in post-surgical stimulation of proliferation of breast carcinoma cells.

Emerging technologies may facilitate progress on both ERBB2 typing and target discovery. Among these, DNA microarrays are currently prominent; they provide massive parallel quantification of mRNA expression levels for thousands of genes in a sample (15, 16, for recent reviews). Several reports have shown that this technology can be used to improve the prognostic classification of breast cancers (17-24). 217 breast carcinomas have been analyzed using DNA microarrays containing ˜9,000 spotted cDNA clones. Our aim was to identify differences in gene expression patterns between ERBB2-negative and ERBB2-positive breast tumors. We have identified a series of 37 discriminator genes/mRNA/ESTs called “ERBB2 gene expression signature,” the expression of which was able to distinguish ERBB2-negative and positive samples. This signature was independently validated by correlative IHC and FISH analyses. Among the genes included in the signature were potential additional targets, such as GATA4.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 represents the supervised classification of 145 breast tumors using ERBB2 gene expression signature. Top panel: The ERBB2 IHC status (HerceptTest) for each tumor sample is shown: a white square indicates sample scored 3+ and a black square indicates sample scored 0-1+. Bottom panel: Expression patterns of 37 cDNA clones in the 145 samples. Each row represents a gene and each column represents a sample. Tumor samples are numbered from 1 to 145. Genes (right of panel) are referenced by their HUGO abbreviation. Each cell in the matrix represents the expression level of a transcript in a single sample relative to its median abundance across all samples and is depicted according to a color scale shown at the bottom. Red and green indicate expression levels respectively above and below the median. The magnitude of deviation from the median is represented by the color saturation. Grey indicates missing data.

FIGS. 2a-2b represent the validation of the ERBB2 gene expression signature by supervised classification of thirty-seven genes/ESTs from an independent series of breast cancer samples. FIG. 2a shows the expression data of 54 additional breast cancers (validation set). Genes/ESTs located on 17q are marked with “*.” FIG. 2b shows the expression data of 16 breast cancer cell lines. For both FIGS. 2a and 2b, the top panel shows the ERBB2 status for each cell line: a white square indicates amplification and/or high mRNA expression of the ERBB2 gene and a black square indicates no amplification and no overexpression. In the bottom panel, each row represents a gene and each column represents a sample. Genes (right of panel) are referenced by their HUGO abbreviation. Red and green indicate expression levels respectively above and below the median. The magnitude of deviation from the median is represented by the color saturation. Grey indicates missing data.

FIG. 3a contains photomicrographs of tissue microarray sections, showing protein expression by hematoxylin and eosin staining (top) or immuno-histochemical staining (bottom). FIG. 3b represents the analysis of ERBB2 gene copy number in breast tumors using fluorescence in situ hybridization on tissue microarray sections.

FIG. 4a represents an unsupervised classification of 159 breast tumors using hierarchical clustering of 159 breast tumors and 37 clones from the ERBB2 gene expression signature. Each row represents a clone and each column represents a sample. Expression level of each gene in a single sample is relative to its median abundance across all samples and is depicted according to a color scale shown at the bottom. Red and green indicate expression levels respectively above and below the median. The magnitude of deviation from the median is represented by the color saturation. Grey indicates missing data. FIG. 4b is a magnification of the dendrogram from the left side of FIG. 4a.

FIG. 5 is a partial chromosome map showing localization of the genes from chromosome 17q12-24 region which are represented on the DNA microarrays. Genes upregulated in the ERBB2 gene signature are indicated in bold. “@” indicates a gene cluster.

FIG. 6 contains representative Herceptest™ results for assessing HER-2/neu Status in patients.

FIGS. 7a and 7b represents an unsupervised hierarchical classification of 159 breast tumors defining an ERBB2 gene expression signature performed as in FIG. 4a, on the basis of 24 clones identified by an iterative approach.

FIG. 8 represents validation of the 24 clone (gene) signature presented in FIG. 7 on an independent set of 54 samples, performed as in FIGS. 2a and 2b.

### SUMMARY

We provide a “gene expression signature” (also referred to as “GES”) that can identify ERBB2 alteration in breast tumors, as well as enhance current understanding of the role of ERBB2 in mammary oncogenesis. The gene expression signature contains genes that are neighbors of ERBB2 on 17q12, and includes potential regulators and/or downstream effectors of ERBB2 (e.g., GATA4) and eventual targets (e.g., cadherin, integrins). The gene expression signature can be used both for breast tumor management in clinical settings and as a research tool, in academic laboratories.

We thus provides a method for analyzing differential gene expression associated with breast tumor, based on the analysis of the over- or under-expression of polynucleotide sequences in a sample or cell line. The analysis comprises the detection of the over-expression of at, least one, preferably at least two, more preferably three or all, polynucleotide sequence(s), subsequence(s) or complement(s) thereof, selected from at least each of predefined polynucleotide sequences sets consisting of:

Set 1: SEQ ID NOS. 73, 74, 75, 76, 77 (ERBB2);

Set 4: SEQ ID NOS. 78, 79, 80 (GATA4); and

Set 5: SEQ ID NOS. 41, 42, 43 (CDH15).

We also provide a method for analyzing differential gene expression associated with breast tumor, based on the analysis of the over- or under-expression of polynucleotide sequences in a sample or cell line. This analysis includes the detection of the over-expression or under-expression of at least one, preferably at least two, more preferably three or all, polynucleotide sequence(s), subsequence(s) or complement(s) thereof, selected from each of predefined polynucleotide sequences sets consisting of Set 1: SEQ ID NO. 73, 74, 75, 76, 77 (ERBB2), Set 2: SEQ ID NO. 28, 29, 30 (GRB7), Set 3: SEQ ID NO. 83, 84, 85 (NR1D1), Set 4: SEQ ID NO. 78, 79, 80 (GATA4), Set 5: SEQ ID NO. 41, 42, 43 (CDH15), Set 6: SEQ ID NO. 16, 17 (LTA), Set 7: SEQ ID NO. 86, 87, 116 (MAP2K6), Set 8: SEQ ID NO. 54, 55, 113 (PECAM1), Set 9: SEQ ID NO. 44, 45 (PPARBP), Set 13: SEQ ID NO. 10 (LOC148696), Set 18: SEQ ID NO. 24, 25 (STAT3), Set 20: SEQ ID NO. 36, 37, 38 (CDKL5), Set 21: SEQ ID NO. 46, 47, 48 (CSTA), Set 22: SEQ ID NO. 52, 53, 115 (ITGB3), Set 23: SEQ ID NO. 56, 57, 58 (MKI67), Set 24: SEQ ID NO. 59, 60, 61 (PBEF), Set 27: SEQ ID NO. 88, 89, 90 (ITGA2), Set 28: SEQ ID NO. 11 (ESTAA878915), SET 29: SEQ ID NO. 1, 2, 3 (JDP1), SET 35: SEQ ID NO. 67, 68, 69 (FLJ10.193), SET 36: SEQ ID NO. 70, 71, 72 (ESR1), SET 43: SEQ ID NO. 104, 105, 106 (DAXX), SET 47: SEQ ID NO. 114, and SET 48: SEQ ID NO. 117, 118 (C17ORF37).

We further provide a polynucleotide library useful for the molecular characterization of a breast cancer, comprising or corresponding to a pool of polynucleotide sequences which are over- or under-expressed in breast tissue.

We still further provide a method for analyzing differential gene expression associated with breast tumor, including a) obtaining nucleic acids from a breast tissue sample from a patient, b) reacting the nucleic acids sample obtained in step (a) with a polynucleotide library or array, and c) detecting the reaction product of step (b).

We yet further provide to a method for analyzing differential gene expression associated with breast tumor, including a) obtaining proteins from a breast tissue sample from a patient, and b) measuring in the sample the level of proteins corresponding to proteins coded by a polynucleotide library or array.

We also further provide a method for treating a patient with a breast cancer, including (i) the implementation of a method for analyzing differential gene expression associated with breast tumor on a sample from the patient, and (ii) determining a treatment for this patient based on the analysis of differential gene expression profile.

### DETAILED DESCRIPTION

As used herein, a disease, disorder, e.g., tumor or condition “associated with” an aberrant expression of a nucleic acid refers to a disease, disorder, e.g., tumor or condition in a subject which is caused by, contributed to by, or causative of an aberrant level of expression of a nucleic acid.

As used herein, the term “subsequence” refers to any part of said polynucleotide sequence that is less than the entire polynucleotide sequence, and which would be also suitable to perform the method of analysis. A person skilled in the art can choose the position and length of a subsequence by applying routine experiments. For example, a subsequence of a polynucleotide can be any contiguous sequence of at least about 10, about 25, about 50, about 100, about 200, about 300, about 400, about 800, or about 1,000 nucleotides. Examples of such subsequences are given in Table 1 below, under the heading “Seq3′” or “Seq5′”.

The over- or under-expression of a given polynucleotide sequence, subsequence or complement thereof can be determined by any known method, such as disclosed in PCT patent application WO 02103320, the entire disclosure of which is herein incorporated by reference. Suitable methods can comprise the detection of difference in the expression of the polynucleotide sequences in relation to at least one control. Said control can comprise, for example, polynucleotide sequence(s) from sample of the same patient or from a pool of ERBB2+ or ERBB2− patients, or polynucleotide sequences selected from among reference sequence(s) which may already be known to be over- or under-expressed. The expression level of said control polynucleotide sequences can be an average or an absolute value of the expression of reference polynucleotide sequences. The values for control polynucleotide expression can be processed in order to accentuate the difference relative to the expression of the polynucleotide sequences.

The analysis of the over- or under-expression of polynucleotide sequences can be carried out on sample such as biological material derived from any mammalian cells, including cell lines, xenografts, and human tissues (preferably breast tissue), etc. The method can be performed on any sample from a patient or an animal (for example for veterinary applications or preclinical trials).

More particularly, we provide a method for analyzing differential gene expression associated with breast tumors, based on the analysis of the over- or under-expression of polynucleotide sequences on a sample or cell line. The analysis comprises the detection of the over-expression of at least one, preferably at least two, more preferably three or all, polynucleotide sequence(s), subsequence(s) or complement(s) thereof, selected from each of at least the predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 3: SEQ ID NO. 83, 84, 85 (NR1D1);

Set 4: SEQ ID NO. 78, 79, 80 (GATA4); and

Set 5: SEQ ID NO. 41, 42, 43 (CDH15).

The method can further comprise at least one of the following embodiments:

The detection of the over-expression of at least one, preferably at least two, more preferably three or all, polynucleotide sequence(s), subsequence(s) or complement(s) thereof, selected from each one of predefined polynucleotide sequences sets consisting of:

Set 6: SEQ ID NO. 16, 17 (LTA);

Set 7: SEQ ID NO. 86, 87, 116 (MAP2K6); and

Set 8: SEQ ID NO. 54, 55, 113 (PECAM1).

The detection of the over-expression of at least one, preferably at least two, more preferably three or all, polynucleotide sequence(s), subsequence(s) or complement(s) thereof from each one of predefined polynucleotide sequences sets consisting of:

Set 9: SEQ ID NO. 44, 45 (PPARBP);

Set 10: SEQ ID NO. 33, 34, 35 (PPP1R1B); and

Set 11: SEQ ID NO. 39, 40 (RPL19).

The detection of the over-expression of at least one, preferably at least two, more preferably three or all, polynucleotide sequence(s); subsequence(s) or complement(s) thereof, from each of predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 3: SEQ ID NO. 83, 84, 85 (NR1D1);

Set 4: SEQ ID NO. 78, 79, 80 (GATA4);

Set 5: SEQ ID NO. 41, 42, 43 (CDH15);

Set 6: SEQ ID NO. 16, 17 (LTA);

Set 7: SEQ ID NO. 86, 87, 116 (MAP2K6);

Set 8: SEQ ID NO. 54, 55, 113 (PECAM1);

Set 9: SEQ ID NO. 44, 45 (PPARBP);

Set 10: SEQ ID NO. 33, 34, 35 (PPP1R1B);

Set 11: SEQ ID NO. 39, 40 (RPL19);

Set 12: SEQ ID NO. 4, 5, 6 (PSMB3);

Set 13: SEQ ID NO. 10 (LOC148696);

Set 14: SEQ ID NO. 12, 13 (NOL3/loc283849);

Set 15: SEQ ID NO. 14, 15 (ITGA2B);

Set 16: SEQ ID NO. 18, 19 (NFKBIE);

Set 17: SEQ ID NO. 22, 23 (PADI2);

Set 18: SEQ ID NO. 24, 25 (STAT3);

Set 19: SEQ ID NO 26, 27 (OAS2);

Set 20: SEQ ID NO. 36, 37, 38 (CDKL5);

Set 21: SEQ ID NO. 46, 47, 48 (CSTA);

Set 22: SEQ ID NO. 52, 53, 115 (ITGB3);

Set 23: SEQ ID NO. 56, 57, 58 (MKI67);

Set 24: SEQ ID NO. 59, 60, 61 (PBEF);

Set 25: SEQ ID NO. 62, 63, 64 (FADS2);

Set 26: SEQ ID NO. 81, 82 (LOX);

Set 27: SEQ ID NO. 88, 89, 90 (ITGA2); and

Set 28: SEQ ID NO. 11 (ESTAA878915).

The under-expression of at least one, preferably at least two, more preferably three or all, polynucleotide sequence(s), subsequence(s) or complement(s) thereof, from each one of predefined polynucleotide sequences sets consisting of:

SET 29: SEQ ID NO. 1, 2, 3 (JDP1);

SET 30: SEQ ID NO. 7, 8, 9 (NAT1);

SET 31: SEQ ID NO. 20, 21 (CELSR2);

SET 32: SEQ ID NO. 31, 32 (ESTN33243);

SET 33: SEQ ID NO. 49, 50, 51 (SCUBE2);

SET 34: SEQ ID NO. 65, 66 (ESTH29301);

SET 35: SEQ ID NO. 67, 68, 69 (FLJ10193); and

SET 36: SEQ ID NO. 70, 71, 72 (ESR1).

According to another embodiment, the method comprises the detection of the over- or under-expression of at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 3: SEQ ID NO. 83, 84, 85 (NR1D1);

Set 4: SEQ ID NO. 78, 79, 80 (GATA4);

Set 5: SEQ ID NO. 41, 42, 43 (CDH15);

Set 6: SEQ ID NO. 16, 17 (LTA);

Set 7: SEQ ID NO. 86, 87, 116 (MAP2K6);

Set 8: SEQ ID NO. 54, 55, 113 (PECAM1);

Set 9: SEQ ID NO. 44, 45 (PPARBP);

Set 10: SEQ ID NO. 33, 34, 35 (PPP1R1B);

Set 11: SEQ ID NO. 39, 40 (RPL19);

Set 13: SEQ ID NO. 10 (LOC148696);

Set 14: SEQ ID NO. 12, 13 (NOL3/loc283849);

Set 15: SEQ ID NO. 14, 15 (ITGA2B);

Set 16: SEQ ID NO. 18, 19 (NFKBIE);

Set 18: SEQ ID NO. 24, 25 (STAT3);

Set 19: SEQ ID NO. 26, 27 (OAS2);

Set 20: SEQ ID NO. 36, 37, 38 (CDKL5);

Set 21: SEQ ID NO. 46, 47, 48 (CSTA);

Set 22: SEQ ID NO. 52, 53, 115 (ITGB3);

Set 23: SEQ ID NO. 56, 57, 58 (MKI67);

Set 24: SEQ ID NO. 59, 60, 61 (PBEF);

Set 26: SEQ ID NO. 81, 82 (LOX);

Set 27: SEQ ID NO. 88, 89, 90 (ITGA2);

SET 29: SEQ ID NO. 1, 2, 3 (JDP1);

SET 33: SEQ ID NO. 49, 50, 51 (SCUBE2);

SET 34: SEQ ID NO. 65, 66 (ESTH29301);

SET 35: SEQ ID NO. 67, 68, 69 (FLJ10193); and

SET 36: SEQ ID NO. 70, 71, 72 (ESR1).

By “over- or under-expression” of a polynucleotide sequence, it is meant that over-expression of certain sequences are detected simultaneously to the under-expression of others sequences. “Simultaneously” means concurrent with or within a biologically or functionally relevant period of time during which the over-expression of a sequence may be followed by the under-expression of another sequence; or conversely, e.g., because expression of both polynucleotide sequences are directly or indirectly correlated.

In a further embodiment, we provide a method for analyzing differential gene expression associated with breast tumors, based on the analysis of the over- or under-expression of polynucleotide sequences in a sample or cell line, said analysis comprising:

the detection of the over-expression of at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 6: SEQ ID NO. 16, 17 (LTA);

Set 23: SEQ ID NO. 56, 57, 58 (MKI67); and

the detection of the under-expression of at least one, preferably at least two or three, polynucleotide sequence(s), subsequence(s) or complement(s) thereof, selected from SET 36: SEQ ID NO. 70, 71, 72 (ESR1).

In a further embodiment, we provide a method for analyzing differential gene expression associated with breast tumors based on the analysis of the over- or under-expression of polynucleotide sequences on a sample or cell line, said analysis comprising the detection of the over-expression or under-expression of at least one, preferably at least two, three or all, polynucleotide(s), subsequence(s) or complement(s) thereof, selected from each of predefined polynucleotide sequences sets consisting of:

Set 1: SEQ ID NO. 75, 76, 77 (ERBB2);

Set 2: SEQ ID NO. 28, 29, 30 (GRB7);

Set 4: SEQ ID NO. 78, 79, 80 (GATA4);

Set 5: SEQ ID NO. 41, 42, 43 (CDH15);

SET 31: SEQ ID NO. 20, 21 (CELSR2);

SET 36: SEQ ID NO. 70, 71, 72 (ESR1); and

SET 48: SEQ ID NO. 117, 118 (C17ORF37).

In a particular embodiment this method comprises:

the detection of the over-expression of at least one preferably at least two, more preferably three or all, polynucleotide sequence(s), subsequence(s) or complement(s) thereof, selected from each of predefined polynucleotide sequences sets consisting of:

Set 1: SEQ ID NO. 75, 76, 77 (ERBB2);

Set 2: SEQ ID NO. 28, 29, 30 (GRB7);

Set 4: SEQ ID NO. 78, 79, 80 (GATA4);

Set 5: SEQ ID NO. 41, 42, 43 (CDH15); and

the detection of the under-expression of at least one, preferably at least two, more preferably three or all, polynucleotide sequence(s), subsequence(s) or complement(s) thereof, selected from each of predefined polynucleotide sequences sets consisting of:

SET 31: SEQ ID NO. 20, 21 (CELSR2);

SET 36: SEQ ID NO. 70, 71, 72 (ESR1); and

SET 48: SEQ ID NO. 117, 118 (C17ORF37).

In a further embodiment, we provide a method for analyzing differential gene expression associated with breast tumors based on the analysis of the over or under expression of polynucleotide sequences in a sample or cell line, said analysis comprising the detection of the over-expression of under-expression of at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 3: SEQ ID NO. 83, 84, 85 (NR1D1);

Set 4: SEQ ID NO. 78, 79, 80 (GATA4);

Set 5: SEQ ID NO. 41, 42, 43 (CDH15);

Set 6: SEQ ID NO. 16, 17 (LTA);

Set 7: SEQ ID NO. 86, 87, 116 (MAP2K6);

Set 8: SEQ ID NO. 54, 55, 113 (PECAM1);

Set 9: SEQ ID NO. 44, 45 (PPARBP);

Set 13: SEQ ID NO. 10 (LOC148696);

Set 18: SEQ ID NO. 24, 25 (STAT3);

Set 20: SEQ ID NO. 36, 37, 38 (CDKL5);

Set 21: SEQ ID NO. 46, 47, 48 (CSTA);

Set 22: SEQ ID NO. 52, 53, 115 (ITGB3);

Set 23: SEQ ID NO. 56, 57, 58 (MKI67);

Set 24: SEQ ID NO. 59, 60, 61 (PBEF);

Set 27: SEQ ID NO. 88, 89, 90 (ITGA2);

Set 28: SEQ ID NO. 11 (ESTAA878915);

SET 29: SEQ ID NO. 1, 2, 3 (JDP1);

SET 35: SEQ ID NO. 67, 68, 69 (FLJ10193);

SET 36: SEQ ID NO. 70, 71, 72 (ESR1);

SET 43: SEQ ID NO. 104, 105, 106 (DAXX);

SET 47: SEQ ID NO. 114; and

SET 48: SEQ ID NO. 117, 118 (C17ORF37).

In another embodiment this method comprises:

the detection of the over-expression of at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2);

Set 2: SEQ ID NO. 28, 29, 30 (GRB7);

Set 3: SEQ ID NO. 83, 84, 85 (NR1D1);

Set 4: SEQ ID NO. 78, 79, 80 (GATA4);

Set 5: SEQ ID NO. 41, 42, 43 (CDH15);

Set 6: SEQ ID NO. 16, 17 (LTA);

Set 7: SEQ ID NO. 86, 87, 116 (MAP2K6);

Set 8: SEQ ID NO. 54, 55, 113 (PECAM1);

Set 9: SEQ ID NO. 44, 45 (PPARBP);

Set 13: SEQ ID NO. 10 (LOC148696);

Set 18: SEQ ID NO. 24, 25 (STAT3);

Set 20: SEQ ID NO. 36, 37, 38 (CDKL5);

Set 21: SEQ ID NO. 46, 47, 48 (CSTA);

Set 22: SEQ ID NO. 52, 53, 115 (ITGB3);

Set 23: SEQ ID NO. 56, 57, 58 (MKI67);

Set 24: SEQ ID NO. 59, 60, 61 (PBEF);

Set 27: SEQ ID NO. 88, 89, 90 (ITGA2);

Set 28: SEQ ID NO. 11 (ESTAA878915);

SET 47: SEQ ID NO. 114;

SET 48: SEQ ID NO. 117, 118 (C17ORF37); and

the detection of the under-expression of at least one, preferably at least two, more preferably three or all, polynucleotide sequence(s), subsequence(s) or complement(s) thereof, selected from each of predefined polynucleotide sequences sets consisting of:

SET 29: SEQ ID NO. 1, 2, 3 (JDP1);

SET 35: SEQ ID NO. 67, 68, 69 (FLJ10193);

SET 36: SEQ ID NO. 70, 71, 72 (ESR1); and

SET 43: SEQ ID NO. 104, 105, 106 (DAXX).

In another embodiment, this method further comprises:

the detection of the over-expression of at least one, preferably at least two, more preferably three or all, polynucleotide sequence(s), subsequence(s) or complement(s) thereof, selected from each of predefined polynucleotide sequences sets consisting of:

SET 38: SEQ ID NO. 94, 95 (B3GNT3);

SET 40: SEQ ID NO. 99; and

SET 44: SEQ ID NO. 107, 108 (ACTR1A); and

the detection of the under-expression of at least one, preferably at least two, more preferably three or all, polynucleotide sequence(s), subsequence(s) or complement(s) thereof, selected from each of predefined polynucleotide sequences sets consisting of:

SET 31: SEQ ID NO. 20, 21 (CELSR2);

SET 33: SEQ ID NO. 49, 50, 51 (SCUBE2);

SET 37: SEQ ID NO. 91, 92, 93 (RHOBTB3);

SET 39: SEQ ID NO. 96, 97, 98 (NUDT14);

SET 41: SEQ ID NO. 100, 101 (CASKIN1);

SET 42: SEQ ID NO. 102, 103 (KIF5C);

SET 45: SEQ ID NO. 109, 110, 111 (MAPT); and

SET 46: SEQ ID NO. 112.

The number of sequences according to the various embodiments can vary in the range of from 1 to the total number of sequences described therein; e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115 or 120 sequences.

The number of sets according to the various embodiments can vary in the range of from 1 to the total number of sets described therein; e.g., 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 sets.

Table 1 hereafter displays a library of polynucleotide sequences of SEQ ID NO. 1 to SEQ ID NO. 118 above. Table 1 indicates the name of the gene with its gene symbol, its clone reference (Image, or Ipsogen in italics) and for each gene the relevant sequence(s) defining the set (identification numbers: SEQ ID NO.). We conveniently define the nucleotide sequences by reference to different sets, but can also define the polynucleotide sequences by the name of the gene or subsequences thereof.

TABLE 1

| | Clone | | Seq3′ | Seq5′ | Ref |
|---|---|---|---|---|---|
| Gene | Image | | SEQ | SEQ | SEQ |
| symbol | Or Ipsogen | Name | ID NO. | ID NO. | ID NO. |
| JDP1 | 120138 | j domain containing protein 1 | 1 | 2 | 3 |
| PSMB3 | 145275 | proteasome (prosome, macropain) | 4 | 5 | 6 |
| | | subunit, beta type, 3 | | | |
| NAT1 | 145894 | n-acetyltransferase 1 (arylamine n- | 7 | 8 | 9 |
| | | acetyltransferase) | | | |
| LOC148696 | 1467504 | hypothetical protein loc148696 | 10 | | |
| ESTAA878915 | 1493187 | sapiens, clone image: 4831215, mrna | 11 | | |
| NOL3/ | 150483 | nucleolar protein 3 (apoptosis | 12 | | 13 |
| loc283849 | | repressor with card domain) | | | |
| ITGA2B | 1506558 | integrin, alpha 2b (platelet | 14 | | 15 |
| | | glycoprotein iib of iib/iiia complex, | | | |
| | | antigen cd41b) | | | |
| LTA | 1524491 | lymphotoxin alpha (tnf superfamily, | 16 | | 17 |
| | | member 1) | | | |
| NFKBIE | 1573311 | nuclear factor of kappa light | 18 | | 19 |
| | | polypeptide gene enhancer in b-cells | | | |
| | | inhibitor, epsilon | | | |
| CELSR2 | 175103 | cadherin, egf lag seven-pass g-type | 20 | | 21 |
| | | receptor 2 (flamingo homolog, | | | |
| | | drosophila) | | | |
| PADI2 | 180060 | peptidyl arginine deiminase, type ii | 22 | | 23 |
| STAT3 | 1950914 | signal transducer and activator of | 24 | | 25 |
| | | transcription 3 (acute-phase response | | | |
| | | factor) | | | |
| OAS2 | | 2′-5′-oligoadenylate synthetase 2, | | 26 | 27 |
| | | 69/71 kDa, transcript variant 2 | | | |
| GRB7 | 236059 | growth factor receptor-bound protein 7 | 28 | 29 | 30 |
| ESTN33243 | 270561 | sapiens cdna flj33383 fis, clone | 31 | 32 | |
| | | brace2006514. | | | |
| PPP1R1B | 277173 | protein phosphatase 1, regulatory | 33 | 34 | 35 |
| | | (inhibitor) subunit 1b (dopamine and | | | |
| | | camp regulated phosphoprotein, | | | |
| | | darpp-32) | | | |
| CDKL5 | 301018 | cyclin-dependent kinase-like 5 | 36 | 37 | 38 |
| RPL19 | 321041 | ribosomal protein 119 | | 39 | 40 |
| CDH15 | 327684 | cadherin 15, m-cadherin (myotubule) | 41 | 42 | 43 |
| PPARBP | 33696 | ppar binding protein | 44 | | 45 |
| CSTA | 345957 | cystatin a (stefin a) | 46 | 47 | 48 |
| SCUBE2 | 346321 | signal peptide, cub domain, egf-like 2 | 49 | 50 | 51 |
| ITGB3 | 0000143 | integrin, beta 3 (platelet glycoprotein | | 52, 115 | 53 |
| | | IIIa, antigen CD61) | | | |
| PECAM1 | 0000133 | platelet/endothelial cell adhesion | | 54, 113 | 55 |
| | | molecule (CD31 antigen) | | | |
| MKI67 | 428545 | antigen identified by monoclonal | 56 | 57 | 58 |
| | | antibody ki-67 | | | |
| PBEF | 488548 | pre-b-cell colony-enhancing factor | 59 | 60 | 61 |
| FADS2 | 51069 | fatty acid desaturase 2 | 62 | 63 | 64 |
| ESTH29301 | 52616 | homo sapiens transcribed sequence | 65 | 66 | |
| | | with weak similarity to protein | | | |
| | | ref: np_060265.1 (h. sapiens) | | | |
| | | hypothetical protein flj20378 [homo | | | |
| | | sapiens] | | | |
| FLJ10193 | 52635 | hypothetical protein flj10193 | 67 | 68 | 69 |
| ESR1 | 725321 | estrogen receptor 1 | 70 | 71 | 72 |
| ERBB2 | 726223 | v-erb-b2 erythroblastic leukemia viral | 73 | 74 | 75 |
| | | oncogene homolog 2, | | | |
| | | neuro/glioblastoma derived oncogene | | | |
| | | homolog (avian) | | | |
| ERBB2 | 756253 | v-erb-b2 erythroblastic leukemia viral | 76 | 77 | 75 |
| | | oncogene homolog 2, | | | |
| | | neuro/glioblastoma derived oncogene | | | |
| | | homolog (avian) | | | |
| GATA4 | 781738 | gata binding protein 4 | 78 | 79 | 80 |
| LOX | 789069 | lysyl oxidase | 81 | | 82 |
| NR1D1 | 795330 | nuclear receptor subfamily 1, group d, | 83 | 84 | 85 |
| | | member 1 | | | |
| MAP2K6 | 0000170 | mitogen-activated protein | | 86, 116 | 87 |
| | | kinasekinase 6, transcript variant 1 | | | |
| ITGA2 | 811740 | integrin, alpha 2 (cd49b, alpha 2 | 88 | 89 | 90 |
| | | subunit of vla-2 receptor) | | | |
| RHOBTB3 | 147138 | rho-related btb domain containing 3 | 91 | 92 | 93 |
| B3GNT3 | 150897 | udp-glcnac:betagal beta-1,3-n- | 94 | 95 | |
| | | acetylglucosaminyltransferase 3 | | | |
| NUDT14 | 152718 | nudix (nucleoside diphosphate linked | 96 | 97 | 98 |
| | | moiety x)-type motif 14 | | | |
| | 159538 | | | 99 | |
| CASKIN1 | 166862 | cask interacting protein 1 | 100 | | 101 |
| KIF5C | 278430 | kinesin family member 5c | 102 | 103 | |
| DAXX | 292042 | death-associated protein 6 | 104 | 105 | 106 |
| ACTR1A | 342342 | arp1 actin-related protein 1 homolog | 107 | | 108 |
| | | a, centractin alpha (yeast) | | | |
| MAPT | 50764 | microtubule-associated protein tau | 109 | 110 | 111 |
| | 52898 | | 112 | | |
| | 0000135 | | | 114 | |
| C17ORF37 | 0000367 | chromosome 17 open reading frame | | 117 | 118 |
| | | 37 | | | |

We provide a method in which the differential gene expression corresponds to an alteration of ERBB2 gene expression of some or all of the polynucleotide sequences from Table 1, or subsequences or complements thereof, in breast tumor and/or an alteration of an ER gene expression in breast tumor.

The detection of over- or under-expression of polynucleotide sequences according to the method can be carried out by any suitable technique, for example by FISH or IHC. It can be performed, for example, on nucleic acids obtained from a breast tissue sample or from a tumor cell line.

In one embodiment, the polynucleotides, or subsequences or complements thereof, are immobilized on DNA microarrays.

The detection of over- or under-expression of polynucleotide sequences according to the method can also be carried out at the protein level, for example, by detecting proteins expressed from nucleic acid in a breast tissue sample.

We provide particularly a method for monitoring the treatment of a patient with a breast cancer comprising the implementation of the above methods on nucleic acids or protein in a breast tissue sample from said patient.

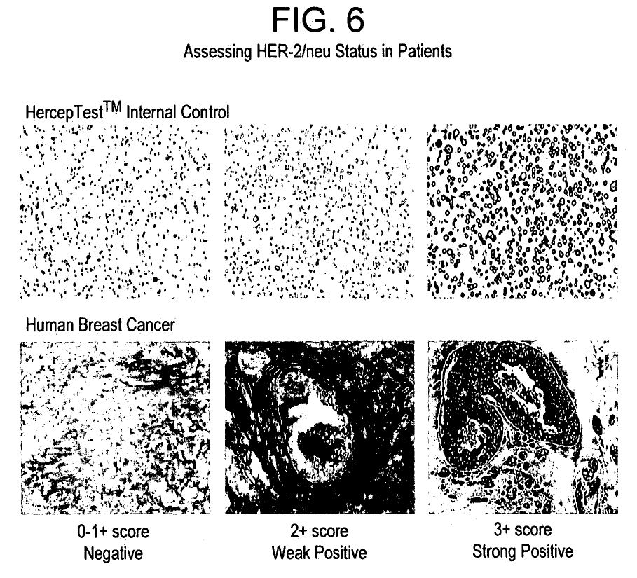

Advantageously, the method is performed on patient scoring +2 with the HercepTest™ (see FIG. 6).

Also advantageously, the method is performed on patients to determine their need to be pre-treated with ERBB2 antagonist, e.g., Herceptin™ (trastuzumab), before surgical removal of ERBB2 positive primary breast tumors. Treatment with ERBB2 inhibitor such as Herceptin™ before ablation could reduce tumor proliferation and metastatic risk stimulated by surgical resection.

We further provide a polynucleotide library useful for the molecular characterization of a breast cancer, comprising or corresponding to a pool of polynucleotide sequences over- or under-expressed in breast tissue. In one embodiment, the pool comprises or corresponds to at least one, preferably at least two, more preferably three or all, polynucleotide sequence(s), subsequence(s) or complement(s) thereof, selected from each of predefined polynucleotide sequences sets consisting of:

### Set 1: SEQ ID NO. 73, 74, 75, 76, 77 (ERBB2); Set 4: SEQ ID NO. 78, 79, 80 (GATA4); Set 5: SEQ ID NO. 41, 42, 43 (CDH15), or

### Set 1: SEQ ID NO. 73, 74, 75, 76, 77 (ERBB2); Set 2: SEQ ID NO. 28, 29, 30 (GRB7); Set 3: SEQ ID NO. 83, 84, 85 (NR1 D1); Set 4: SEQ ID NO. 78, 79, 80 (GATA4); Set 5: SEQ ID NO. 41, 42, 43 (CDH15).

The pool can also comprise at least one, preferably at least two, more preferably three or all, polynucleotide sequence, subsequence or complement thereof, selected in each of predefined polynucleotide sequences sets of at least one of the following groups:

### Set 6: SEQ ID NO. 16, 17 (LTA); Set 7: SEQ ID NO. 86, 87, 116 (MAP2K6); Set 8: SEQ ID NO. 54, 55, 113 (PECAM1);

### Set 9: SEQ ID NO. 44, 45 (PPARBP); Set 10: SEQ ID NO. 33, 34, 35 (PPP1R1B); Set 11: SEQ ID NO. 39, 40 (RPL19);

Set 12: SEQ ID NO. 4, 5, 6 (PSMB3); Set 13: SEQ ID NO. 10 (LOC148696); Set 14: SEQ ID NO. 12, 13 (NOL3/loc283849); Set 15: SEQ ID NO. 14, 15 (ITGA2B); Set 16: SEQ ID NO. 18, 19 (NFKBIE); Set 17: SEQ ID NO. 22, 23 (PADI2); Set 18: SEQ ID NO. 24, 25 (STAT3); Set 19: SEQ ID NO. 26, 27 (OAS2); Set 20: SEQ ID NO. 36, 37, 38 (CDKL5); Set 21: SEQ ID NO. 46, 47, 48 (CSTA); Set 22: SEQ ID NO. 52, 53, 115 (ITGB3); Set 23: SEQ ID NO. 56, 57, 58 (MKI67); Set 24: SEQ ID NO. 59, 60, 61 (PBEF); Set 25: SEQ ID NO. 62, 63, 64 (FADS2); Set 26: SEQ ID NO. 81, 82 (LOX); Set 27: SEQ ID NO. 88, 89, 90 (ITGA2); SET 28: SEQ ID NO. 11 (ESTAA878915); and
SET 29: SEQ ID NO. 1, 2, 3 (JDP1); SET 30: SEQ ID NO. 7, 8, 9 (NAT1); SET 31: SEQ ID NO. 20, 21 (CELSR2); SET 32: SEQ ID NO. 31, 32 (ESTN33243); SET 33: SEQ ID NO. 49, 50, 51 (SCUBE2); SET 34: SEQ ID NO. 65, 66 (ESTH29301); SET 35: SEQ ID NO. 67, 68, 69 (FLJ10193); SET: SEQ ID NO. 70, 71, 72 (ESR1).

A specific polynucleotide library useful for the molecular characterization of a breast cancer comprises or corresponds to a pool of polynucleotide sequences over- or under-expressed in breast tissue, said pool comprising or corresponding to at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2); Set 2: SEQ ID NO. 28, 29, 30 (GRB7); Set 3: SEQ ID NO. 83, 84, 85 (NR1D1); Set 4: SEQ ID NO. 78, 79, 80 (GATA4); Set 5: SEQ ID NO. 41, 42, 43 (CDH15); Set 6: SEQ ID NO. 16, 17 (LTA); Set 7: SEQ ID NO. 86, 87, 116 (MAP2K6); Set 8: SEQ ID NO. 54, 55, 113 (PECAM1); Set 9: SEQ ID NO. 44, 45 (PPARBP); Set 10: SEQ ID NO. 33, 34, 35 (PPP1R1B); Set 11: SEQ ID NO. 39, 40 (RPL19); Set 13: SEQ ID NO. 10 (LOC148696); Set 14: SEQ ID NO. 12, 13 (NOL3/loc283849); Set 15: SEQ ID NO. 14, 15 (ITGA2B); Set 16: SEQ ID NO. 18, 19 (NFKBIE); Set 18: SEQ ID NO. 24, 25 (STAT3); Set 19: SEQ ID NO. 26, 27 (OAS2); Set 20: SEQ ID NO. 36, 37, 38 (CDKL5); Set 21: SEQ ID NO. 46, 47, 48 (CSTA); Set 22: SEQ ID NO. 52, 53, 115 (ITGB3); Set 23: SEQ ID NO. 56, 57, 58 (MKI67); Set 24: SEQ ID NO. 59, 60, 61 (PBEF); Set 26: SEQ ID NO. 81, 82 (LOX); Set 27: SEQ ID NO. 88, 89, 90 (ITGA2); SET 29: SEQ ID NO. 1, 2, 3 (DPI); SET 33: SEQ ID NO. 49, 50, 51 (SCUBE2); SET 34: SEQ ID NO. 65, 66 (ESTH29301/NA); SET 35: SEQ ID NO. 67, 68, 69 (FLJ10193); and SET 36: SEQ ID NO. 70, 71, 72 (ESR1).

A further specific polynucleotide library useful for the molecular characterization of a breast cancer comprises or corresponds to a pool of polynucleotide sequences over or under expressed in breast tissue, said pool comprising or corresponding to at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2); Set 2: SEQ ID NO. 28, 29, 30 (GRB7); Set 6: SEQ ID NO. 16, 17 (LTA); Set 23: SEQ ID NO. 56, 57, 58 (MKI67); and SET 36: SEQ ID NO. 70, 71, 72 (ESR1).

A further specific polynucleotide library useful for the molecular characterization of a breast cancer-comprises or corresponds to a pool of polynucleotide sequences over- or under-expressed in breast tissue, said pool comprising or corresponding to at least one, preferably at least two, more preferably three or all, polynucleotide sequence(s), subsequence(s) or complement(s) thereof, selected from each of predefined polynucleotide sequences sets consisting of:

Set 1: SEQ ID NO. 75, 76, 77 (ERBB2); Set: SEQ ID NO. 28, 29, 30 (GRB7); Set 4: SEQ ID NO. 78, 79, 80 (GATA4); Set 5: SEQ ID NO. 41, 42, 43 (CDH15); SET 31: SEQ ID NO. 20, 21 (CELSR2); SET 3: SEQ ID NO. 70, 71, 72 (ESR1); SET 48: SEQ ID NO. 117, 118 (C17ORF37.)

A further specific polynucleotide library useful for the molecular characterization of a breast cancer comprises or corresponds to a pool of polynucleotide sequences over- or under-expressed in breast tissue, said pool comprising or corresponding to at least one, preferably at least two, more preferably three or all, polynucleotide sequence(s), subsequence(s) or complement(s) thereof, selected from each of predefined polynucleotide sequences sets consisting of:

Set 1: SEQ ID NO. 73, 74, 75, 76, 77 (ERBB2); Set 2: SEQ ID NO. 28, 29, 30 (GRB7); Set 3: SEQ ID NO. 83, 84, 85 (NR1D1); Set 4: SEQ ID NO. 78, 79, 80 (GATA4); Set 5: SEQ ID NO. 41, 42, 43 (CDH15); Set 6: SEQ ID NO. 16, 17 (LTA); Set 7: SEQ ID NO. 86, 87, 116 (MAP2K6); Set 8: SEQ ID NO. 54, 55, 113 (PECAM1); Set 9: SEQ ID NO. 44, 45 (PPARBP); Set 13: SEQ ID NO. 10 (LOC148696); Set 18: SEQ ID NO. 24, 25 (STAT3); Set 20: SEQ ID NO. 36, 37, 38 (CDKL5); Set 21: SEQ ID NO. 46, 47, 48 (CSTA); Set 22: SEQ ID NO. 52, 53, 115 (ITGB3); Set 23: SEQ ID NO. 56, 57, 58 (MKI67); Set 24: SEQ ID NO. 59, 60, 61 (PBEF); Set 27: SEQ ID NO. 88, 89, 90 (ITGA2); Set 28: SEQ ID NO. 11 (ESTAA878915); SET 29: SEQ ID NO. 1, 2, 3 (JDP1); SET 35: SEQ ID NO. 67, 68, 69 (FLJ10193); SET 36: SEQ ID NO. 70, 71, 72 (ESR1); SET 43: SEQ ID NO. 104, 105, 106 (DAXX); SET 47: SEQ ID NO. 114; and SET 48: SEQ ID NO. 117, 118 (C17ORF37).

This pool may further comprise at least one, preferably at least two, more preferably three or all, polynucleotide sequence(s), subsequence(s) or complement(s) thereof, selected from each of predefined polynucleotide sequences sets consisting of: SET 31: SEQ ID NO. 20, 21 (CELSR2); SET 33: SEQ ID NO. 49, 50, 51 (SCUBE2); SET 37: SEQ ID NO. 91, 92, 93 (RHOBTB3); SET 38: SEQ ID NO. 94, 95 (B3GNT3); SET 39: SEQ ID NO. 96, 97, 98 (NUDT14); SET 40: SEQ ID NO. 99; SET 41: SEQ ID NO. 100, 101 (CASKIN1); SET 42: SEQ ID NO. 102, 103 (KIF5C); SET 44: SEQ ID NO. 107, 108 (ACTR1A); SET 45: SEQ ID NO. 109, 110, 111 (MAPT); and SET 46: SEQ ID NO. 112.

The term “pool”, as used herein, refers to a number of sequences that may vary in a range of from 1 to the total number of polynucleotide sequences, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115 or 120 sequences.

The polynucleotide libraries can be immobilized on a solid support to form an array. The solid support can, for example, be selected from the group consisting of nylon membrane, nitrocellulose membrane, glass slide, glass beads, membranes on glass support or a silicon chip.

Thus, a method comprises:

obtaining nucleic acids from a breast tissue sample from a patient; and

reacting said nucleic acids obtained in step (a) with a polynucleotide library; and

detecting the reaction product of step (b).

The polynucleotide sample can be labeled, e.g., before reaction step (b), and the label of the polynucleotide sample can be selected from the group consisting of radioactive, colorimetric, enzymatic, molecular amplification, bioluminescent or fluorescent labels. For example, a preferred label can be selected from the group consisting of biotin and digoxygenin.

The method can further comprise obtaining a control sample comprising polynucleotides, reacting said control sample with a polynucleotide library, detecting a control sample reaction product and comparing the amount of said polynucleotide sample reaction product to the amount of said control sample reaction product.

By “nucleic acids” is meant polynucleotides; e.g., isolated polynucleotides, such as deoxyribonucleic acid (DNA), and, where appropriate, ribonucleic acid (RNA). “Nucleic acids” should also be understood to include, as equivalents, analogs of RNA or DNA made from nucleotide analogs, and, as applicable to the embodiment being described, single (sense or antisense) and double-stranded polynucleotides: ESTs, chromosomes, cDNAs, mRNAs, and rRNAs are representative examples of molecules that may be referred to as nucleic acids. DNA can be obtained, for example, from said nucleic acids sample and RNA can be obtained, for example, by transcription of said DNA. In addition, mRNA can be isolated from said nucleic acids sample and cDNA can be obtained by reverse transcription of said mRNA.

In a further embodiment, a method can be performed at the protein level. Such a method can comprise:

obtaining proteins from a breast tissue sample from a patient; and

measuring proteins in the sample obtained in step (a), in which the level of proteins in the sample corresponds to proteins coded by a polynucleotide library. It is understood that the proteins can be obtained directly from the sample; e.g., by standard extraction or isolation techniques or can be obtained by translation of mRNA obtained from the samples.

Our methods are useful for detecting, diagnosing, staging, monitoring, predicting, or preventing conditions associated with breast cancer. It is particularly useful for predicting clinical outcome of breast cancer and/or predicting occurrence of metastatic relapse and/or determining the stage or aggressiveness of a breast disease in at least about 50%, e.g., at least about 55%, e.g., at least about 60%, e.g., at least about 65%, e.g., at least about 70%, e.g., at least about 75%, e.g., at least about 80%, e.g., at least about 85%, e.g., at least about 90%, e.g., at least about 95%, e.g., about 100% of the patients. The methods are also useful for selecting more appropriate doses and/or schedule for administering chemotherapeutics and/or biopharmaceuticals and/or radiation therapy to circumvent toxicities in a patient.

By “aggressiveness of a breast disease” is meant, e.g., cancer growth rate or potential to metastasize; a so-called “aggressive cancer” will grow or metastasize more rapidly than a non-aggressive cancer, or significantly affect overall health status and quality of life.

By “predicting clinical outcome” is meant, e.g., the ability for a skilled artisan to classify patients into at least two prognostic classes (good vs. poor) showing significantly different long-term Metastasis Free Survival (MFS).

We also provide a method for treating a patient with a breast cancer, comprising i) implementing a method of analyzing differential gene expression profile on a sample from said patient, and ii) determining a treatment for this patient based on the analysis of differential gene expression profile obtained with said method. “Treating” encompasses palliative care as well as ameliorating at least one symptom of the condition or disease.

The methods can achieve high specificity and sensitivity level of at least about 80%, e.g., about 85%, e.g., about 90%, e.g., about 93%, e.g., about 95% e.g., about 97%, e.g., about 99% in predicting the clinical outcome, in predicting occurrence of metastatic relapse, or determining the stage or aggressiveness of breast cancer.

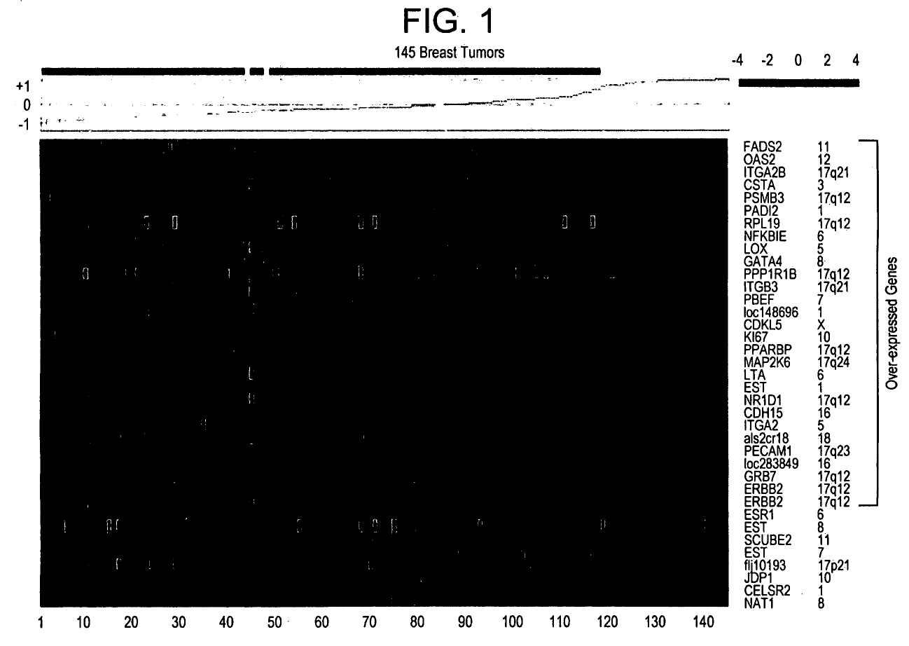

FIG. 1 represents the supervised classification of 145 breast tumors using ERBB2 gene expression signature. Shown is the classification of the learning sample set (145 cases) by supervised analysis on the basis of 37 clones identified by iterative approach and defining the ERBB2 gene expression signature (GES). Expression patterns of 37 cDNA clones in 145 samples is shown in the bottom panel. Each row represents a gene and each column represents a sample. Tumor samples are numbered from 1 to 145. Genes (right of panel) are referenced by their HUGO abbreviation as used in “Locus Link” (maintained by the U.S. National Center for Biotechnology Information (NCBI) of the National Library of Medicine) and their chromosomal location (including which arm for chromosome 17). “EST” (Expressed Sequenced Tag) is used for clones without similarity with known gene or protein. Samples are ordered according to the correlation of their expression profile with the average profile of the ERBB2-positive group, and genes are ordered by their discriminating score. Each cell in the matrix represents the expression level of a transcript in a single sample relative to its median abundance across all samples, and is depicted according to a color scale shown at the bottom. Red and green indicate expression levels respectively above and below the median. The magnitude of deviation from the median is represented by the color saturation. Grey indicates missing data. The ERBB2 IHC status (HerceptTest) for each tumor sample is shown in the top panel: a white square indicates sample scored 3+ and a black square indicates sample scored 0-1+.

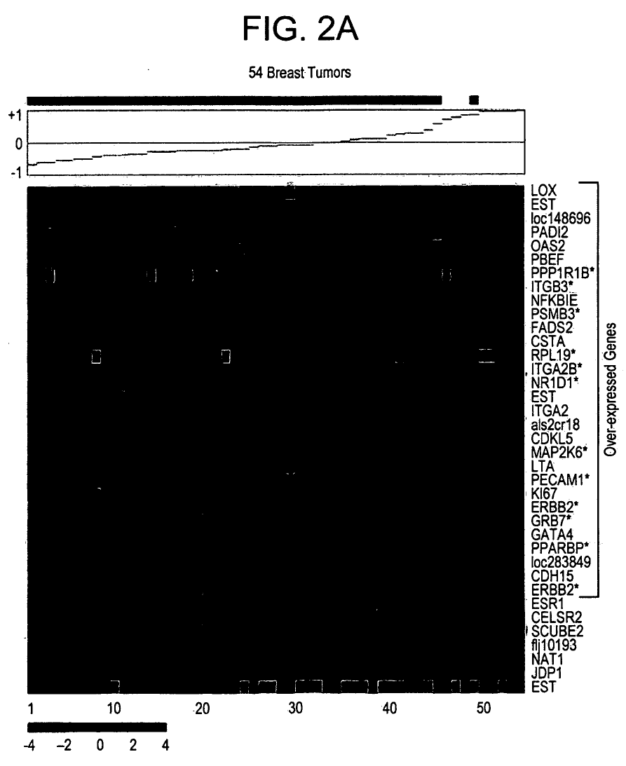

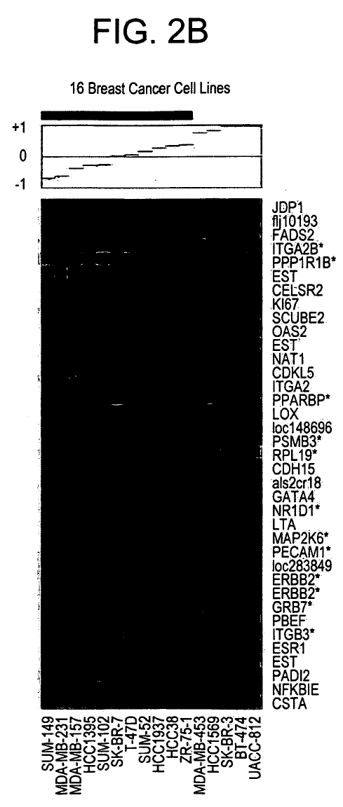

FIG. 2 represents the validation of the ERBB2 gene expression signature. A ERBB2 gene expression signature (37 genes/ESTs) was used for classifying independent series of breast cancer samples. FIG. 2a is a supervised analysis as in FIG. 1, applied to the expression data of 54 additional breast cancers (validation set). Genes/ESTs located on 17q are marked with “*.” FIG. 2b is a supervised analysis as in FIG. 1, applied to the expression data of 16 breast cancer cell lines. The ERBB2 status for each cell line is shown in the top panel of both FIGS. 2a and 2b: a white square indicates amplification and/or high mRNA expression of the ERBB2 gene and black square indicates no amplification and no over-expression.

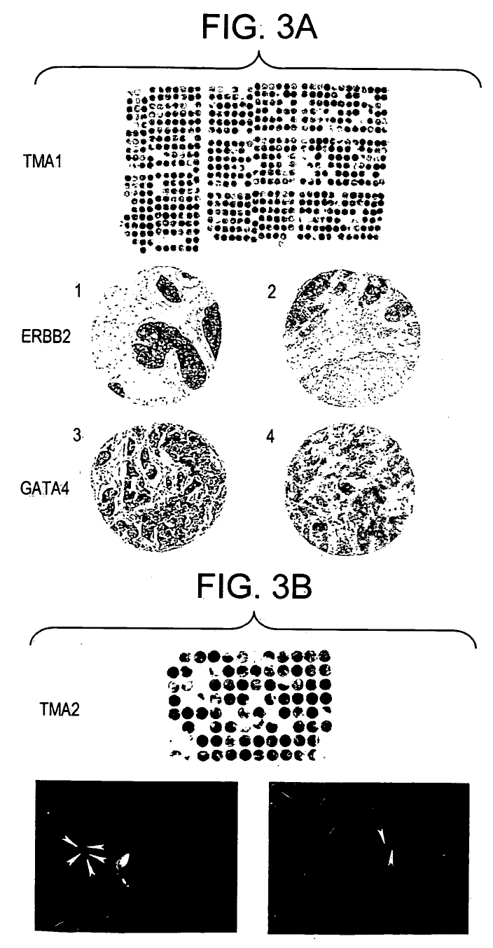

FIG. 3a represents the analysis of protein expression using immunohistochemistry on tissue microarray sections. “TMA1” indicates a hematoxylin-eosin staining (H & E) of paraffin block section (25×30 mm) from TMA1 containing 552 tumors and control samples. Examples of IHC staining are indicated by the numbers 1-4. Section 1 shows a sample with ERBB2 expression equal to 3+ and section 2 shows a sample with no detected ERBB2 expression. Section 3 shows a sample with GATA4 expression equal to Q=300, and section 4 shows a sample with no GATA4 expression.

FIG. 3b represents the analysis of ERBB2 gene copy number in breast tumors using fluorescence in situ hybridization (FISN) on tissue microarray sections. “TMA2” indicates H & E staining of paraffin block section (25×30 mm) from TMA2-containing 94 tumors. Below the TMA2 section, two sections of invasive breast carcinomas are shown, the first with ERBB2 amplification and the second with normal gene copy number. Red dots (arrows) represent ERBB2 copies and green dots represent centromere 17, on interphase, chromosomes.

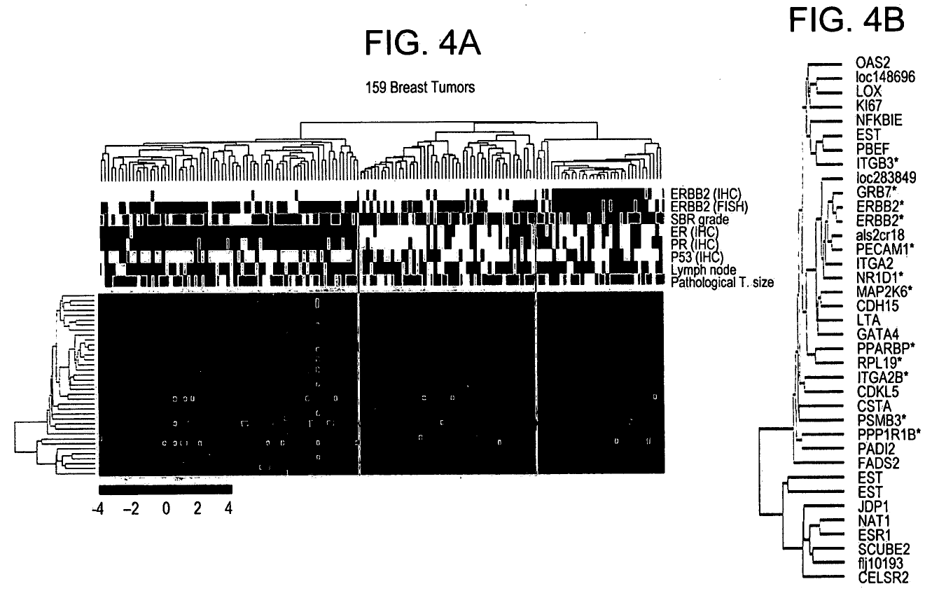

FIG. 4 represents an unsupervised hierarchical classification of 159 breast tumors using genes from the ERBB2 gene expression signature. In FIG. 4a, hierarchical clustering of 159 breast tumors and 37 clones from the ERBB2 gene expression signature is shown. Each row represents a clone and each column represents a sample. Expression level of each gene in a single sample is relative to its median abundance across all samples, and is depicted according to a color scale shown at the bottom. Red and green indicate expression levels respectively above and below the median. The magnitude of deviation from the median is represented by the color saturation. Grey indicates missing data. Dendrograms of samples (above data matrix) and genes (to the left of matrix) represent overall similarities in gene expression profiles. The orange vertical lines mark the subdivision into three main tumor groups; they are represented in the branches of dendrogram in green (A), black (B) and red (C), respectively. The dendrogram of genes is magnified to show detail in FIG. 4b. Between the dendtogram of samples and the data matrix relevant histoclinical data for the 159 tumors are represented according to a grey color ladder: ERBB2 IHC status (HercepTest: 0-1+, white; 2+, light grey; 3+, black; unavailable, dark grey), ERBB2 FISH status (negative, white; positive, black; unavailable, dark grey), SBR grade (1, white; 2, light grey; 3, black; unavailable, dark grey), ER, PR and P53 IHC status (negative, white; positive, black; unavailable, dark grey), axillary lymph node invasion (negative, white; positive, black), pathological size of tumors (pT1, white; pT2, light grey; pT3, black). In FIG. 4b, the dendrogram of genes is referenced by their HUGO abbreviation. Genes/ESTs located on 17q are marked with “*.” The “ERBB2 cluster” (red branches) and the “ER cluster” (green branches) respectively contain the ERBB2 and ESR1 genes.

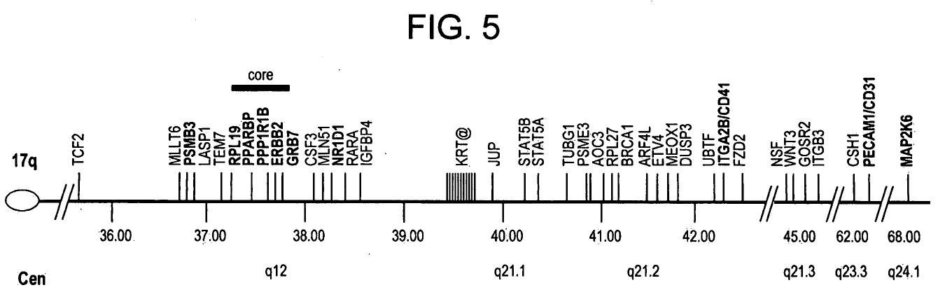

FIG. 5 shows localization of genes from the chromosome region 17q12-24 represented on the DNA microarray. Genes whose expression were upregulated in the ERBB2 breast cancer series as identified by supervised analysis of gene expression profiling using DNA microarrays are indicated in bold. The other genes indicated were represented on the microarray but were not found in the ERBB2 signature. The list of genes is not thorough for genes located outside 17q12. From several studies, a “core” of genes can be identified that is almost always co-over-expressed with ERBB2. In FIG. 5, “@” means gene cluster.

FIG. 6 represents Herceptest™ assessing HER-2/neu status in patients.

Herceptest™ is the first co-approval of molecular diagnostic and therapeutic agent consisting of: stringent standardization of HER-2/neu antisera and IHC protocols; increased awareness for scrupulous quality control; standardized, universal controls, and system for pathological scoring; results interpreted by pathologists specifically trained to consistently score Her-2 immunostaining (ie. use of reference laboratories).

As shown in FIG. 6, a negative result on the Herceptest™ would depict no staining or faint membrane staining in more than 10 percent of the tumor cells. Only part of the membrane stains.

A weak positive result on the Herceptest™ would depict weak to moderate complete membrane staining in more than 10 percent of the tumor cells.

A strong positive on the Herceptest™ result would depict a strong complete membrane staining in more than 10 percent of the tumor cells.

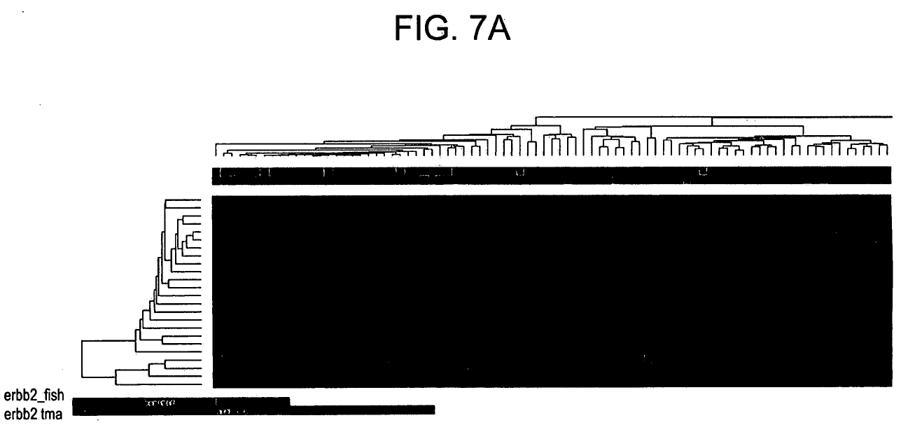

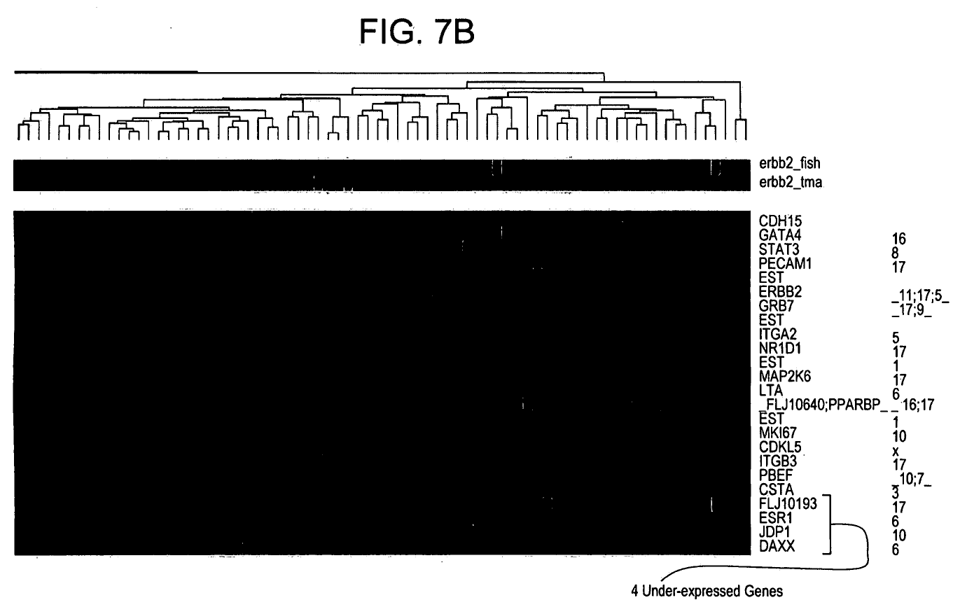

FIG. 7 represents another unsupervised hierarchical classification of 159 breast tumors as in FIG. 1 (split in two parts 7a and 7b due to figure length) on the basis of 24 clones identified by iterative approach and defining ERBB2 gene expression signature (GES). Under-expressed genes are indicated; the others are over-expressed.

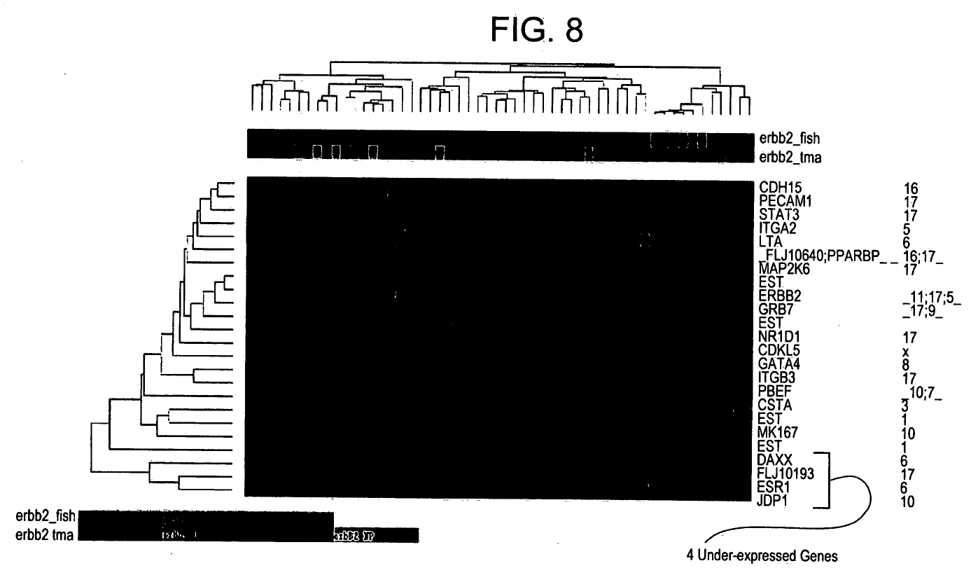

FIG. 8 represents validation of the 24 clones (genes) signature presented in FIG. 7 on an independent set of 54 samples. Under-expressed genes are indicated; the others are over-expressed.

The row/column representation principle in FIGS. 7 and 8 is as described for FIG. 1.

We thus provide a set of genes, the analysis of which produces a gene expression profile that can discriminate between ERBB2+ and ERBB2− breast tumors. Content of the signature

The identity of the discriminator genes gives insight into the underlying biological mechanisms associated with ERBB2 status and with the aggressive phenotype of ERBB2+ breast cancers. They also provide new diagnostic, prognostic and predictive factors, as well as new therapeutic targets.

Twenty-nine genes/ESTs were significantly over-expressed in ERBB2+ tumors. Without wishing to be bound by any theory, their co-expression may indicate co-amplification (same chromosomal location), regulation by ERBB2, coregulation by common factors or association with unknown phenotypic feature of disease. In addition to ERBB2 itself, there were 6 genes from region q12 of chromosome 17 in the signature (See FIG. 1); the 6 genes are all located within less than one megabase on either side of ERBB2, defining a small “core” region of co-expressed—probably co-amplified—genes (See FIG. 5). Again without wishing to be bound by any theory, over-expression of these genes with ERBB2 may be associated with DNA amplification of the 17q12 amplicon; nevertheless, the functional affect of overabundant transcripts of these genes may impact on the clinical outcome in breast cancer patients. Indeed, this may be the case, for example, for GRB7 or PPARBP. GRB7, a tyrosine kinase cytoplasmic adaptor substrate, has been implicated with different partners in integrin-mediated cell migration (33). PPARBP has been shown to downregulate P53-dependent apoptosis (34). Other genes from the microarray and located on 17q but further apart from ERBB2 were not found in the signature, except for ITGA2B/CD41, ITGB3/CD61, PECAM1/CD31, and MAP2K6. Again, without wishing to be bound by any theory, over-expression of these genes may not be due to increased ERBB2 gene copy number per se but may be triggered by intense ERBB2 signaling; it might also be due to the presence of other telomeric, 17q-associated amplicons (35, 36). ITGA2, whose gene is not on 17q, was also over-expressed in ERBB2+ tumors. There may be a other loci whose transcription is coordinately increased because the corresponding proteins belong to the same network. In total, four genes expressed in endothelial cells and platelets (encoding three integrins ITGA2, ITGA2B, ITGB3, and an adhesion molecule of the Ig family PECAM1) were over-expressed in ERBB2+ tumors (however, not all integrin genes from 17q present on the microarray were over-expressed since ITGA3 was not).

Collectively, these data indicate that neoangiogenesis and/or changes in blood vessel organization may play an important role in the pathogenesis of these tumors, and confirm that Herceptin and anti-cancer agents have an additive and/or synergistic activity. Other genes in the near vicinity of ERBB2 locus may be co-amplified with ERBB2 gene but may not be expressed due to the absence of an appropriate promoter or to repression. It is known that only a small proportion of genes from a given amplicon are over-expressed (37).

Other over-expressed genes were not located on chromosome arm 17q. CDH15, also called M-Cadherin or myotubule cadherin, is expressed in myoepithelial cells and may play a role in the muscle-like differentiation of these cells. Again, without wishing to be bound by any theory, this might suggest that ERBB2+ tumors have a certain degree of myoepithelial differentiation; alternatively they may be characterized by a high degree of dedifferentiation with appearance of new markers (this may also be true for other RNAs such as PECAM1).

An interesting finding was GATA4, whose co-expression with ERBB2 was validated at the protein level. This gene codes for a transcription factor of the GATA family (38). It is expressed in adult vertebrate heart, gut epithelium, and gonads. GATA4 is essential for cardiovascular development (39, 40), and regulates genes critical for myocardial differentiation and function. Likewise, ERBB2 is essential for heart development (41; reviewed in 42). Therefore, without wishing to be bound by any theory, ERBB2 may exert some of its downstream effects through GATA4 or, alternatively, GATA4 may stimulate ERBB2 gene transcription by positive feedback regulation.

MAP2K6 is also strongly expressed in cardiac muscle (43). The major adverse effect of Herceptin is cardiotoxicity (44). Investigation of the functional relationship between ERBB2, GATA4 and MAP2K6 may enhance current understanding of cardiotoxicities associated with ERBB2 antagonists, and contribute to design ways to circumvent this side-effect. Activation of GATA4 is thought to occur through RHO GTPases (45, 46), which are also central to the physiologic and pathophysiologic functions of integrins and cadherins (47, for review).

The data disclosed herein also shows variability in ERBB2 and/or GATA4 gene expression, and ERBB2 and GATA4 co-variability may potentially serve as an indicator of patient risk for cardiotoxicity by Herceptin treatment. Therefore, we also provide to a method for determining the risk of averse cardiovascular secondary events for patients treated with Herceptin, comprising the analysis of the differential expression GATA4 gene from a sample or cell line of said patient.

As discussed above, we provide a method comprising the detection of the over- or under-expression of at least one, preferably at least two or more preferably three, polynucleotide sequence(s), subsequence(s) or complement(s) thereof, selected from each of at least one predefined polynucleotide sequence sets consisting of:

Set 1: SEQ ID NO. 73, 74, 75, 76, 77 (ERBB2); and

Set 4: SEQ ID NO. 78, 79, 80 (GATA4).

The MKI67 gene encodes the proliferation marker Ki67/MIB1. This marker was upregulated in ERBB2+ samples, suggesting that ERBB2+ tumors are proliferative tumors. Immunohistochemical results on ˜250 TMA1 tumors for ERBB2 and Ki67 stainings showed that expression of both proteins were correlated, confirming gene clustering at the protein level, in agreement with recent reports (48, 49). The over-expression of the CSTA gene, which encodes cystatin A, a cysteine protease inhibitor of the stefin family that acts as endogenous inhibitor of cathepsins, can be put in perspective with the finding of Oh et al. (14) on the downregulation of cathepsin D in ERBB2-transfected MCF-7 cells. Finally, the presence of genes encoding two structurally-related factors, lymphotoxin A (LTA) and preB-cell colony-enhancing factor (PBEF), and NFKBIE imply that specific immune and inflammatory mechanisms may be associated with ERBB2+ tumors.

Five genes with known function were downregulates in ERBB2-positive tumors. Interestingly, one of these was ESR1, which encodes estrogen receptor α, an important modulator of hormone dependent mammary oncogenesis. It is recognized that most ERBB2-amplified tumors are ER-negative and are resistant to hormone therapy (50-53). Moreover, an interplay between ERBB2 and ER pathways has been demonstrated (54). SCUBE2, a gene encoding a secreted protein with an EGF-like domain (55), and CELSR2, which encodes a non-classical cadherin, might have antagonistic regulatory roles of ERBB2 activities at the cell membrane. SCUBE2 and NAT1 were associated to ESR1 in a gene expression signature associated with ER positivity (24).

### 2) ERBB2 and Microarrays

Several recent gene expression studies have addressed the issue of ERBB2 status and function in breast cancer. Most of them used cancer cell lines, and others included tissue samples.

An early large-scale study of the ERBB2 amplicon was done on 7 breast tumor cell lines by Kauraniemi et al. (30) using a custom-made cDNA microarray that included 217 clones from chromosome region 17q12. ERBB2, GRB7, PPP1R1B were consistently over-expressed when amplified, in conjunction with other genes that were not on microarray constructed from libraries. Willis et al. (56) used a commercially available oligonucleotide chip (Affymetrix GeneChip Hu35K) to study mRNA from 12 breast tumors and from two cell lines also typed using comparative genomic hybridization. A total of 20 known genes showed significant over-expression in tumors with gains of region 17q12-23. These included ERBB2, GRB7, PPARBP, but also MLLT6. KRT10 and TUBG1 that were not identified in the gene signature.

Wilson et al. (31) used a commercially available “breast specific” nylon microarray with ˜5,000 cDNAs to study cell lines and two sets of 5 ERBB2-positive and negative pooled breast tumors. Only few genes from 17q were among the upregulated genes; these included RPL19 and LASP1. Dressman et al. (57) studied 34 tumors and established a gene expression signature specific of ERBB2+ samples that contained several 17q genes including GRB7, NR1D1, PSMB3, and RPL19. Sorlie et al. (24) have also defined ERBB2+ signature with five genes from 17q12, including ERBB2 and GRB7.

Genes located in the vicinity of ERBB2 are frequently co-upregulated following DNA amplification. This phenomenon is less marked for genes located further apart from ERBB2, which may be included only when the amplification affects a large segment from the region. Some of the genes close to ERBB2 did not appear in the present signature, whereas they were upregulated in other studies (i.e. LASP1, MLLT6). This may be due to a different proportion of tumors with variably-sized amplicons in the analyzed panels.

While amplification of region 17q12-21 can affect ERBB2 chromosomal neighbors, ERBB2 protein over-expression can affect downstream targets and possibly also upstream regulators via positive feedback regulatory mechanisms. Balance in cadherins and integrins and functional processes associated with cell-matrix adhesive systems seem particularly affected in ERBB2-positive tumors (31). This suggests that ERBB2 oncogenic activity may be associated with cell motility, as has been proposed previously (58, 59).

A recent study, using DNA microarrays from the Sanger center containing ˜6,000 unique genes/ESTs, has described the transcriptional changes associated with a series of 61 genes following over-expression of a transfected ERBB2 gene in an immortalized HB4a human mammary luminal epithelial cell line (60). Previously, several studies had identified genes whose transcription is affected by ERBB2 over-expression or amplification using differential screening (14, 61). Some of these genes are located near the ERBB2 locus. The present gene expression signature GES shares no common gene with the list of Kumar-Sinha et al. (62) established in comparing cell lines including ERBB2-transfected cell line; however, a gene related to fatty acid biology, FADS2, is part of the present gene expression signature.

Tiwari et al. (63) reported a relationship between ERBB2, fatty acids and 2′,5′ oligoadenylate synthetases (OAS2), which is included in the present “ERBB2 cluster” (See the figures). Peroxisome proliferator-activated receptors (PPARs) are known regulators of lipid metabolism; their trans-activating capacity depends on the recruitment of auxiliary proteins (64, for review. Modifications of fatty acid metabolism in ERBB2+ tumors may thus be associated with over-expression of PPARBP.

### 3) ERBB2 Signature and Assessment of ERBB2 Status

Alteration of ERBB2 expression is associated with poor prognosis (unfavorable clinical outcome with metastasis and death) and can be countered by a targeted therapy based on a humanized antibody, trastuzumab (Herceptin™). Therefore, the determination of ERBB2 status is important in breast cancer management. Accurate quantitation of ERBB2 expression, however, has proved to be difficult since both IHC and FISH have limitations and can be influenced by many variables (9-13). As a consequence, there is still no consensus on the best method for assessing ERBB2 status. In routine practice, IHC, which more than FISH detects the actual target of Herceptin™, is faster and more economic but highly dependent on fixative conditions, staining procedures, scoring system, quality controls and interlaboratory standardization. In addition, results are often difficult to interpret since a number of cases show only moderate over-expression of the protein and discrepancies in the results are subject to interobserver variability. FISH methods are quantitative and sensitive (65), but are also expensive, time-consuming and require specialized expertise and equipment. Indeed, variable concordance between IHC and FISH have led to the current practice of testing +2 HercepTest patients by both IHC and FISH to making a clinical decisions on whether to recommend treatment with anti-ERBB2 antagonists.

The work carried out shows the potential of DNA microarray-based gene expression profiling to establish ERBB2 status, and to identify among ERBB2 2+ cases those with gene amplification and those without.

Our methods will now be illustrated by the following non-limiting examples.

### Materials and Methods

### 1) Breast Carcinoma Samples

Using DNA microarrays, 217 breast cancer samples obtained from 210 women treated at the Institute Paoli-Calmettes between 1988 and 2001 were studied. Inclusion criteria of samples were: i)—sporadic primary localized breast cancer treated with surgery followed by adjuvant anthracyclin-based chemotherapy, ii)—tumor material quickly dissected and frozen in liquid nitrogen and stored at −160° C. Exclusion criteria included locally advanced or inflammatory or metastatic forms. The main characteristics of patients and tumors are listed in Table 2 below.

TABLE 2

| Characteristic | No (%)* |
|---|---|
| Age, years | |
| median (range) | 53 (29, 83) |
| Histological type | |
| ductal | 166 (76) |
| lobular | 25 (12) |
| mixed | 12 (6) |
| tubular | 4 (2) |
| medullary | 3 (2) |
| other | 4 (2) |
| Axillary lymph node status | |
| negative | 57 (26) |
| positive | 160 (74) |
| Pathological tumor size | |
| pT1 | 59 (27) |
| pT2 | 117 (54) |
| pT3 | 41 (19) |
| SBR grade | |
| I | 32 (15) |
| II | 99 (46) |
| III | 85 (39) |
| Peritumoral vascular invasion | |
| absent | 115 (53) |
| present | 101 (47) |
| ER status (IHC) | |
| negative | 72 (34) |
| positive | 142 (66) |
| PR status (IHC) | |
| negative | 80 (38) |
| positive | 130 (62) |
| ERBB2 status (IHC) | |
| 0-1+ | 162 (78) |
| 2+ | 10 (4) |
| 3+ | 37 (18) |
| P53 status (IHC) | |
| negative | 144 (69) |
| positive | 65 (31) |
| ERBB2 status (FISH) | |
| negative | 38 (56) |
| positive | 30 (44) |

*% of evaluated cases

Immunohistochemical parameters collected included estrogen receptor (ER), progesterone receptor (PR) and P53 status (positivity cut-off values of 1%), and ERBB2 status (0-3+ score as illustrated by the HercepTest kit scoring guidelines). All tumor sections were reviewed de novo by two pathologists prior to analysis, and all samples contained more than 50% tumor cells. The series of 217 samples was divided in two sets: a first set of 163 samples, from which was derived, before supervised analysis, a “learning” set of 145 samples, and a second set of 54 samples designated the “validation” set.

A consecutive series of 552 women with unilateral localized invasive breast carcinomas treated at the Institut Paoli-Calmettes between June 1981 and December 1999 was studied using a first TMA designated TMA1. Of the 552 cases studied, 257 were available for ERBB2, GATA4, ER and Ki67 staining. According to the WHO classification, there were 194 ductal, 26 lobular, 10 tubular, 3 medullary carcinomas and 24 other histological types. The average age at diagnosis was 59 years, median age 60, with a range of 25 to 91 years. A total of 135 tumors were associated with lymph node invasion, and 199 were positive for ER. A set of 94 tumors (chosen within tumors analyzed by DNA microarrays) was included in a second TMA designated TMA2.

### 2) Breast Tumor Cell Lines

Except for SUM-52, SUM-102, and SUM-149 (a gift of S. P. Ethier, Ann Arbor, Mich.) the breast cancer cell lines (BT474, HCC38, HCC1395, HCC1569, HCC1937, MDA-MB-157, MDA-MB-231, MDA-MB453, SK-BR-3, SK-BR-7, T47D, UACC-812, and ZR-75-1) were obtained from the American Type. Culture Collection (ATCC; Rockville, Md.). All cell lines were grown according to the recommendations of the supplier.

### 3) RNA Extraction

Total RNA was extracted from frozen tumor samples and cell lines by standard methods using guanidinium isothiocyanate solution and centrifugation on cesium chloride cushion, as previously described in (25), the entire disclosure of which is herein incorporated by reference. RNA integrity was controlled by electrophoresis on agarose gels and by Agilent analysis (Bioanalyzer, Palo Alto, Calif.) before labeling.

### 4) Construction of DNA Microarrays

PCR products from a total of 9038 Image clones, including 3910 expressed sequenced tags (EST) and 5125 known genes, were spotted on 12×8.5 cm² nylon filters with a Microgrid II robot (Biorobotics Apogent Discoveries). Several controls were included in the microarrays, such as poly(A)⁺ stretches, plant cDNAs, and PCR controls. Microarray spotting and hybridization processes were done as previously described in (19), the entire disclosure of which is herein incorporated by reference.

### 5) DNA Microarray Data Analysis and Statistical Methods

Hybridizations of microarray membranes were done with radioactive [alpha-³³P]-dCTP-labeled probes made from 5 μg of total RNA from each sample according to described protocols. Membranes were then washed, exposed to phosphor-imaging plates and scanned with a FUJI BAS 1500 machine. Signal intensities were quantified with ArrayGauge software (Fuji, Dusseldorf, Germany), normalized for amount of spotted DNA as described in (21) the entire disclosure of which is herein incorporated by references and the variability of experimental conditions using non-linear rank-based methods as described in (26), the entire disclosure of which is herein incorporated by references then log-transformed. We first applied supervised analysis to identify the optimal set of genes which best discriminated between ERBB2-negative and positive breast cancer samples. The positivity cut-off of ERBB2 status was defined by protein expression using IHC (HercepTest™ kit): positive status was defined as 3+ and negative status as 0 or 1+ (See FIG. 6). Analysis was done in two steps: the molecular signature was first derived through training on a set of 145 samples (learning set, including 116 ERBB2-negative and 29 ERBB2-positive samples); samples with ERBB2 status 2+(n=10) or unavailable (n=8) were not included in the supervised analysis. It was then validated on the set of 54 samples (validation set, including 46 ERBB2-negative and 8 ERBB2-positive samples).

ProfileSoftware™ Corporate (Ipsogen, Marseille) was utilized for all analyses. This program uses a discriminating score (DS) (17) combined with iterative random permutation tests. The DS′ was calculated for each gene as DS=(M1−M2)/(S1+S2) where M1 and S1 respectively represent mean and standard deviation of expression levels of the gene in subgroup 1 (ERBB2-positive), and M2 and S2 in subgroup 2 (ERBB2-negative). Statistical confidence levels were estimated by bootstrap resampling as previously described in (27) the entire disclosure of which is herein incorporated by references with a false positive rate of 2/10000.

Briefly, approximately two-thirds (n=106) of the samples from the learning set (n=145) were randomly selected to include at least 20 ERBB2-positive cases. They were then submitted to supervised analysis described above. The process was repeated 30 times (30 randomly defined subgroups of 106 samples), thus generating 30 lists of genes. These lists were then compared and a gene was considered as a discriminator if present in at least 25 gene-lists out of 30; allowing the identification of the most relevant genes, independent of the sample set used.

Unsupervised hierarchical clustering was applied to investigate relationships between samples and relationships between genes identified by supervised analysis. The hierarchical clustering was applied to data log-transformed and median-centred on genes using the ProfileSoftware™ Corporate program (Ipsogen, Marseille) (average linkage clustering using uncentered Pearson correlation as similarity metric) and results were displayed with the same program.

### 6) Construction of Tissue Microarrays

Two TMA, TMA1 (552 samples) and TMA2 (94 samples), were prepared as described in (28) with slight modifications (29) the entire disclosure of which are herein incorporated by reference. For each tumor, a representative tumor area was carefully selected by histopathological analysis of a hematoxylin-eosin stained section of a donor block. Core cylinders (one for each tumor for TMA2 and three for each tumor for TMA1) with a diameter of 0.6 mm for TMA 1 and 2 mm for TMA2, were punched from this area and deposited into a recipient paraffin block using a specific arraying device (Beecher Instruments, Silver Spring, Md.). In addition to tumor tissues, the recipient block also included normal breast and established breast tumor cell lines to serve as internal controls: BT-474 known to have four to eight-fold amplification of the ERBB2 gene, and MCF-7, whose chromosomes 17 each have one copy of the ERBB2 gene (30). Five-μm sections of the resulting array block were mounted onto glass slides and used for IHC (TMA1) and FISH (TMA2) analyses. The reliability of the method was assessed by comparison with conventional sections for the usual prognostic parameters (including estrogen receptor and ERBB2); the value of the kappa test was 0.95 (29).

### 7) Antibodies

The following antibodies were used for IHC: polyclonal antibody anti-ERBB2 (Dako-HercepTest™, Copenhagen, Denmark), used strictly following the guidelines described by the manufacturer; goat polyclonal antibody anti-GATA4 (sc-1237, 1:50 dilution; Santa Cruz Biotechnology, Inc., Santa Cruz, Calif.), anti-MIB1/Ki67 (1:100 dilution, Dako), anti-ER-(clone 6F11, 1:60 dilution, Novocastra Laboratories)

### 8) Immunohistochemistry

IHC was done on five-μm sections of TMA1. Briefly, tissues were deparaffinized in Histolemon (Carlo Erba Reagenti, Rodano, Italy) and rehydrated in graded alcohol. Antigen retrieval was done by incubation at 98° C. in citrate buffer. Slides were transferred to a Dako autostainer, except for Dako-HercepTest™ where guidelines are imposed by the manufacturer. Staining was done at room temperature as follows: after washes in phosphate buffer, endogenous peroxidase activity was quenched by treatment with 0.1% H₂O₂, slides were pre-incubated with blocking serum (Dako Corporation) for 10 min, then incubated with the affinity-purified antibody for one hour. After washes, slides were incubated with biotinylated antibody against rabbit IgG for 20 min followed by streptadivin-conjugated peroxidase (Dako LSAB®2 kit). Immunoreactive complexes were visualized with the peroxidase substrate, diaminobenzidine, counter-stained with hematoxylin, and coverslipped using Aquatex (Merck, Darmstadt, Germany) mounting solution. Slides were evaluated under a light microscope by three pathologists.

Immunoreactivities for GATA4 and ER were classified by estimating the percentage (P) of tumor cells showing characteristic staining (from undetectable level or 0%, to homogenous staining or 100%) and by estimating the intensity (I) of staining (weak staining or 1, moderate staining or 2, strong staining or 3). Results were scored by multiplying the percentage of positive cells by the intensity, i.e. by the so-called quick score (Q) (Q=P×I; maximum=300). For Ki67, only the percentage (P) of tumor cells was estimated, since intensity does not vary and for ERBB2, the status was defined using the Dako scale. Expression levels allowed the tumors to be grouped in two categories: no expression (Q=0 for GATA4 and ER, P<20 for Ki67, and 0/+ for ERBB2), and expression (Q>0 for GATA4 and ER, P>20 for Ki67, and 2+/3+ for ERBB2). The average of the score of a minimum of two core biopsies was calculated for each case of TMA 1.

### 9) ERBB2 Gene Amplification Detected by FISH

FISH for ERBB2 gene amplification was done on TMA2 using the Dako ERBB2 FISH PharmDX™ Kit according to the manufactuter's instructions. In brief, TMA2 sections were baked overnight at 55° C., deparaffinized in Histolemon (Carlo Erba Reagenti, Rodano, Italy), rehydrated in graded alcohol and washed in Dako wash buffet. Slides were pretreated by immersion in Dako pretreatment solution at 97° C. for 10 min and cooled to room temperature. Slides were then washed in Dako wash buffer and immersed in Dako pepsin at room temperature for 10 min. Pepsin was removed with two changes of wash buffer. Slides were dehydrated in graded alcohol. Ten μl of HER2/CEN17 (centromere 17) Probe Mix (Dako) was added to the sample area of each section. Sections were coverslipped and the edges were sealed with rubber cement. Slides were placed on a flat metal surface and heated at 82° C. for 5 min to codenature the probe and target DNA, and transferred to a preheated humidified hybridization chamber to hybridize the probe and DNA for 18 h at 45° C. After hybridization, the rubber cement and the coverslips were removed from the slides. Sections were washed in wash buffet at 65° C. then at room temperature. Slides were dehydrated in graded alcohol and air-dried in the dark. Nuclei were counterstained with 15 μl of DAP1/antifade and coverslipped. Slides were stored at −4° C. in the dark for up to 7 days prior to analysis.

### 10) FISH Scoring

Sections were examined with a fluorescent microscope (Zeiss-Axiophot) using the filter recommended by Dako. The invasive lesion selected for the TMA2 was easily localized under the microscope. Approximately forty malignant, non overlapping cell nuclei were scored for each case, and included and scored only if HER2 and CEN17 signals were clearly detected. A ratio of HER2/CEN17 was calculated for each specimen that met this inclusion criteria. ERBB2 was considered as amplified when the FISH ratio HER2/CEN17 was >=2.0. Each assay was read twice by two observers. Specimens were considered negative when less than 10% of tumor cells showed amplification of ERBB2.

### 11) Statistical Analysis

Correlations between hierarchical clustering-based tumor groups and molecular and histoclinical parameters were investigated by using the Chi² test. All p-values were two-sided at the 5% level of significance. Distributions of molecular markers analyzed by TMA1 were compared using Fisher exact test.

### Results

The mRNA expression profiles from 217 different human breast cancer samples and 16 breast cancer cell lines were determined with cDNA microarrays containing ˜9,000 spotted PCR products from known genes and ESTs. Analysis, both supervised and unsupervised, identified an ERBB2-specific gene expression signature (GES). To further validate this signature, studies were completed by FISH and IHC analyses on breast cancer tissue microarrays.

1) Identification and Validation of an ERBB2 Gene Expression Signature from Tumor Profiling

Supervised analysis was utilized to identify a gene expression signature correlated with ERBB2 status. It was applied to the mRNA expression profiles from 145 randomly chosen breast cancer samples (learning set) by comparing two subgroups defined by their ERBB2 status as determined by standard IHC: samples scoring 0 and 1+ (hereafter designated ERBB2−, 116 samples) were compared to samples scoring 3+ (ERBB2+, 29 samples). Cases with equivocal 2+(n=10) or unavailable (n=8) staining were excluded from analysis. To identify a molecular signature independent from the predefined subgroups of tumors identified by IHC, several different subsets of samples were iteratively defined and supervised analysis was performed on each of these subsets independently. Thirty such iterations were done. The lists of genes identified as significant discriminators (these lists ranged from 80 to 274 clones) were then compared, revealing 37 clones present in at least 25 lists: these clones defined an ERBB2-specific gene expression signature (GES). All of the genes identified in this signature were tag-resequenced to confirm their identity.

FIG. 1 shows the expression pattern of this signature in the 145 breast cancer samples in a color-coded matrix. Tumor samples are classified on the horizontal axis according to their correlation coefficients with the ERBB2+ group. As shown, the resulting discrimination between ERBB2+ and ERBB2− samples was successful. These 37 clones corresponded to 36 unique sequences representing 29 characterized genes (two different clones represented ERBB2) and 7 other sequences or ESTs. Twenty-nine were over-expressed and 8 were under-expressed in ERBB2+ samples. Their chromosomal location is listed in FIG. 1.

Once identified on this set of 145 samples, we validated our ERBB2 GES in an independent set of 54 breast cancer samples (validation set). As shown in FIG. 2a, classification of samples based on the GES successfully classified them according to ERBB2 IHC status with only 1 ERBB2-negative sample misplaced in the ERBB2+ group.

2) Comparative Analysis of ERBB2 Gene Expression Signature of Human Breast Tissues to Breast Cancer Cell Lines

On the Ipsogen DiscoveryChip, a series of 16 breast cancer cell lines were profiled. The cell lines included 5 cell lines (BT474, HCC1569, MDA-MB-453, SK-BR-3 and UACC-812) known to have amplification and/or high mRNA expression of the ERBB2 gene (30, 31). ERBB2 GES successfully separated ERBB2+ and ERBB2-cell lines (FIG. 2b), further validating the discriminator potential of the signature.

Collectively, these analyses demonstrated that the ERBB2 gene expression signature correctly classified breast tumors and cell lines consistent with ERBB2 status evaluated with standard procedure (Herceptest™, Dako Corporation).

3) Analysis of Breast Tumor Samples Using Tissue Microarrays

Significant discriminator genes were further validated by immunohistochemical analysis of their corresponding proteins (FIG. 3a). A total of ˜250 cases from TMA1 were available for the study of ERBB2, ER, GATA4 and Ki67. In ERBB2 GES, ERBB2, GATA4 and Ki67 genes were over-expressed and ESR1 was under-expressed in ERBB2+ samples. These correlations were confirmed at the protein level: over-expression of ERBB2 protein was significantly associated with an upregulation of GATA4 (p<0.001), Ki67 (p<0.025), and with negativity of ER (p<0.0001) (Table 3 hereunder).

TABLE 3

| | ERBB2 | ERBB2 | |
|---|---|---|---|
| | (0-1+) | (2-3+) | |
| | n (%) | n (%) | p-value* |
| GATA4 | | | |
| negative | 169 (90%) | 18 (10%) | |
| positive | 50 (71%) | 20 (29%) | <0.001 |
| Ki67 | | | |
| <20 | 151 (88%) | 21 (12%) | |
| >=20 | 59 (78%) | 17 (22%) | <0.025 |
| ER | | | |
| negative | 27 (60%) | 18 (40%) | |
| positive | 179 (90%) | 20 (10%) | <0.0001 |

*Fisher exact test

We found 40% of ERBB2-positive tumors in ER-negative tumors but only 10% in ER-positive tumors.

A total of 68 (72%) of the 94 samples included in TMA2 were available for FISH analysis of ERBB2 locus. Examples of results are shown in FIG. 3b. Of the 68 cases, 30 displayed ERBB2 amplification whereas 38 were not amplified.

### 4) Classification of Breast Tumors Using ERBB2 Gene Expression Signature

Previous supervised analyses did not include the breast cancer samples scored 2+ for ERBB2 IHC. We reclassified these cases with all 145 samples previously analyzed—which included the 68 cases with available FISH ERBB2 data—by using hierarchical clustering program based on ERBB2 GES. Results are displayed in FIG. 4, which highlights clusters of correlated genes across clusters of correlated samples (n=159, learning set, 2+ samples, and 4 samples with unavailable ERBB2 status). The first large gene cluster contained 29 genes/ESTs, including. ERBB2 (it was designated “ERBB2 cluster”). The second gene cluster was globally anticorrelated with the previous one: it contained 8 genes/ESTs, including ESR1 that codes for estrogen receptor α (it was designated “ER cluster”).

Despite significant transcriptional heterogeneity between tumors for these genes, the combined expression patterns defined at least three clusters of tumors, designated A, B and C. Group A (73 cases, in green) displayed an over-expression of the “ER cluster” and an under-expression of the “ERBB2 cluster” overall compared to groups B and C. Conversely, the “ERBB2 cluster” and the “ER cluster” were upregulated and downregulated in group C samples (36 cases, in red) overall, as compared to other groups. Finally, group B′ (50 cases, in black) displayed an intermediate profile with heterogenous expression of the “ERBB2 cluster” and under-expression of the “ER cluster”.

Correlations of tumor groups as defined by hierarchical clustering with ERBB2 status were analyzed. As expected, group C strongly differed from the other groups with respect to ERBB2 protein expression since 93% of all ERBB2 3+ samples were located in this group. In group C 77% of samples scored 3+, 9% 2+ and 14% 0-1+; in contrast, in groups A and B, these rates were 0% and 5% (3+), 3% and 10% (2+), and 97% and 85% (0-1+) (p<0.0001, Chi² test, A vs B vs C groups), respectively. As expected, there was also a strong correlation between tumor groups and FISH status with most of the FISH positive cases clustered in group C (p<0.0001, Chi² test, A vs B vs C groups). ERBB2 FISH information and IHC status were both available in 64 cases out of 159. Interestingly, the three 2+ tumors located in group C displayed ERBB2 amplification (FISH positive), while the seven 2+ tumors included in group A (2 cases) and group B (5 cases) had no amplification (FISH negative). These results shows that our ERBB2 GES could separate FISH-positive and FISH-negative ERBB2 2+ tumors, providing more specific information than FISH with respect to ERBB2 IHC status (HercepTest™). Indeed, the correlation between GES groups (C samples vs A+B samples) and FISH result (negative vs positive) provided a sensitivity of 90% and a specificity of 88% (concordance in 89% of cases). In comparison, the correlation between IHC-based grouping (0-1+ vs 2-3+) and FISH status showed an equal sensitivity of 90% but a weaker specificity of 76% (concordance in 82% of cases) (Table 4 hereunder).

TABLE 4

FISH status

| | | negative | positive | Total* |
|---|---|---|---|---|
| GES groups | A + B | 30 | 3*** | 33 |
| | C | 4 | 27 | 31 |
| Total* | | 34 | 30 | 64 |
| IHC status** | negative | 26 | 3*** | 29 |
| | positive | 8 | 27 | 35 |
| Total* | | 34 | 30 | 64 |

*considering 64 tumors with data available for IHC, FISH et GES-based grouping;
**negative: 0-1+ and positive, 2-3+;
***two samples are probably false-positive FISH results.

Sensitivity was better for the two-comparisons; as shown in FIG. 4, two samples located in groups A and B and IHC-negative for ERBB2 were FISH-positive; reviewing of the corresponding sections revealed in fact the presence of intra-ductal carcinoma in one case and abundant necrosis in the other case, both of which might have lead to false positive FISH results. Verification using real-time quantitative PCR demonstrated absence of ERBB2 amplification. Taken into account the two samples with false-positive FISH results, the error rate was 5 out of 64 (with 4 false-positive and 1 false-negative) for correlation between our classification and FISH, whereas it was 9 out of 64 for correlation between standard IHC and FISH.

5) Correlation with Histoclinical Parameters

We searched for correlations between tumor groups and relevant molecular and histoclinical parameters of samples. Our GES-based grouping correlated with SBR grade and hormone receptor status, further, albeit indirectly, validating our classification. Group C did not contain grade 1 samples; 44% of samples were grade 2 and 56% were grade 3. In groups A+B, 15% of samples were grade 1, 48% were grade 2 and 37% were grade 3 (p=0.02, Chi-2 test). In group C, samples were likely to be ER-negative (59%), compared with 27% in groups A+B (p=0.001, Chi-2 test). Similarly, although not significant, correlation was found for PR status (p=0.07, Chi² test). No correlation was found with pathological size of tumors, axillary lymph node status and P53 IHC status.

### REFERENCES

1. Slamon D J, Clark G M, Wong S G, Levin W J, Ullrich A, McGuire W L: Human breast cancer: correlation of relapse and survival with amplification of the HER-2/neu oncogene. Science 1987, 235, 177-182.
2. Eccles S A: The role of c-erbB-2/HER2/neu in breast cancer progression and metastasis. J Mammary Gland Biol Neoplasia 2001, 6:393-406.
3. Holbro T, Civenni G, Hynes N E: The ErbB receptors and their role in cancer progression. Exp Cell Res 2003, 284:99-110.
4. Ross J S, Fletcher J A: The HER-2/neu oncogene: prognostic factor, predictive factor and target for therapy. Semin Cancer Biol 1999, 9:125-138.
5. Hayes D F, Thor A D: c-erbB-2 in breast cancer: development of a clinically useful marker. Semin Oncol 2002, 29:231-245.
6. Slamon D J: Herceptin®: increasing survival in metastatic breast cancer. Eur J Oncol Nurs 2000, 4:24-29.
7. Horton J: Trastuzumab use in breast cancer: clinical issues. Cancer Control 2002, 9:499-507.
8. Leyland-Jones B: Trastuzumab: hopes and realities. Lancet Oncol 2002, 3:137-144.
9. Di Leo A, Dowsett M, Horten B, Penault-Liorca F: Current status of HER2 testing. Oncology 2002, 63 Suppl 1:25-32.
10. Rampaul R S, Pinder S E, Gullick-W J, Robertson J F, Ellis 10: HER-2 in breast cancer—methods of detection, clinical significance and future prospects for treatment. Crit Rev Oncol Hematol 2002, 43:231-244.
11. Bilous M, Dowsett M, Hanna W, Isola J, Lebeau A, Moreno A, Penault-Llorca F, Ruschoff J, Tomasic G, Van De Vijver M: Current Perspectives on HER2 Testing: A Review of National Testing Guidelines. Mod Pathol 2003, 16:173-182.
12. Zarbo R J, Hammond M E: Conference summary, Strategic Science symposium. Her-2/neu testing of breast cancer patients in clinical practice. Arch Pathol Lab Med 2003, 127:549-553.
13. Pauletti G, Dandekar S, Rong H, Ramos L, Peng H, Seshadri R, Slamon D J: Assessment of methods for tissue-based detection of the HER-2/neu alteration in human breast cancer: a direct comparison of fluorescence in situ hybridization and immunohistochemistry. J Clin Oncol 2000, 18:3651-3664.
14. Oh J J, Grosshans D R, Wong S G, Slamon D J: Identification of differentially expressed genes associated with HER-2/neu over-expression in human breast cancer cells. Nucleic Acids Res 1999, 27:4008-4017.
15. Bertucci F, Viens P, Hingamp P, Nasser V, Houlgatte R, Birnbaum D: Breast cancer revisited using DNA array-based gene expression profiling. Int J Cancer 2003, 103: 565-571
16. Bertucci F, Viens P, Tagett R, Nguyen C, Houlgatte R, Birnbaum D. DNA arrays in clinical oncology: promises and challenges. Lab Invest 2003, 83:305-316.
17. Golub T R, Slonim D K, Tamayo P, Huard C, Gaasenbeek M, Mesirov J P, Coller H, Loh M L, Downing J R, Caligiuri M A, Bloomfield C D, Lander E S: Molecular classification of cancer: class discovery and class prediction by gene expression monitoring. Science 1999, 286:531-537.
18. Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D. Molecular portraits of human breast tumors. Nature 2000, 406:747-752
19. Bertucci F, Houlgatte R, Benziane A, Granjeaud S, Adelaide J, Tagett R, Loriod B, Jacquemier J, Viens P, Jordan B, Birnbaum D Nguyen C: Expression profiling in primary breast carcinomas using arrays of candidate genes. Hum Mol Genet 2000, 9:2981-2991
20. Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown P O, Botstein D, Eystein Lonning P, Borresen-Dale A L. Gene expression patterns of breast carcinomas distinguish tumor subclasses with clinical implications. Proc Natl Acad Sci USA 2001; 98: 10869-10874.
21. Bertucci F, Nasser V, Granjeaud S, Eisinger F, Adelaide J, Tagett R, Loriod B, Giaconia A, Benziane A, Devilard E, Jacquemier J, Viens P, Nguyen C, Birnbaum D, Houlgatte R: Gene expression profiles of poor prognosis primary breast cancer correlate with survival. Hum Mol Genet 2002, 11: 863-872
22. Van't Veer U, Dai H, van de Vijver M, He Y D, Hart A A, Mao M, Peterse H L, van der Kooy-K, Marton M J, Witteveen A T, Schreiber G J, Kerkhoven R M, Roberts C, Linsley P S, Bernards R, Friend S H: Gene expression profiling predicts clinical outcome of breast cancer. Nature 2002, 415:530-535
23. van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R: A gene-expression signature as a predictor of survival in breast cancer. N Engl J Med 2002, 347:1999-2009
24. Sorlic T, Tibshirani R, Parker J, Hastie T, Marron J S, Nobel A, Deng S, Johnsen H, Pesich R, Geisler S, Demeter J, Perou C M, Lonning P E, Brown P O, Borresen-Dale A L, Botstein D: Repeated observation of breast tumor subtypes in independent gene expression data sets. Proc Natl Acad Sci USA 2003, 100:8418-8423.
25. Theillet C, Adelaide J, Louason G, Bonnet-Dorion F, Jacquemier J, Adnane J, Longy M, Katsaros D, Sismondi P, Gaudray P, Birnbaum D: FGFR1 and PLAT genes and DNA amplification at 8p12 in breast and ovarian cancers. Genes Chromosomes Cancer 1993, 7:219-226.
26. Sabatti C, Karsten S L, Geschwind D H: Thresholding rules for recovering a sparse signal from microarray experiments. Math Biosci 2002, 176:17-34.
27. Magrangeas F, Nasser V, Avet-Loiseau H, Loriod B, Decaux O, Granjeaud S, Bertucci F, Birnbaum D, Nguyen C, Harousseau J L, Bataille R, Houlgatte R, Minvielle S: Gene expression profiling of multiple myeloma reveals molecular portraits in relation to the pathogenesis of the disease. Blood 2003101:4998-5006.
28. Richter J, Wagner U, Kononen J, Fijan A, Bruderer J, Schmid U, Ackerman D, Maurer R, Alund G, Knönagel H, Rist M, Wilber K, Anabitarte M, Hering F, Hardmeier T, Schonenberger A, Flury R, Jager P, Fehr J L, Schrami P, Moch H, Mihatsch M J, Gasser T, Kallioniemi O P, Sauter G: High-throughput tissue microarray analysis of cyclin E gene amplification and over-expression in urinary bladder cancer. Am J Pathol 2000, 157:787-794.
29. Ginestier C, Charafe-Jauffret E, Bertucci F, Eisinger F, Geneix I, Bechlian D, Conte N, Adelaide J, Toiron Y, Nguyen C, Viens P, Mozziconacci M J, Houlgatte R, Birnbaum D, Jacquemier J: Distinct and complementary information provided by use of tissue and DNA microarrays in the study of breast tumor markers. Am J Pathol 2002, 161:1223-1233
30. Kauraniemi P, Barlund M, Monni O, Kallioniemi A: New amplified and highly expressed genes discovered in the ERBB2 amplicon in breast cancer by cDNA microarray. Cancer Res 2001, 61:8235-8240.
31. Wilson K S, Roberts H, Leek R, Harris A L, Geradts J: Differential gene expression patterns in HER2/neu-positive and -negative breast cancer cell lines and tissues. Am J Pathol 2002, 161:1171-1185
32. Revillion F, Bonneterre J, Peyrat J P: ERBB2 oncogene in human breast cancer and its clinical significance. Eur J Cancer 1998, 34:791-808.
33. Shen T L, Han D C, Guan J L: Association of Grb7 with phosphoinositides and its role in the regulation of cell migration. J Biol Chem 2002, 277:29069-29077
34. Frade R, Balbo M, Barel M: RB18A regulates p53-dependent apoptosis. Oncogene 2002, 21:861-866.
35. Andersen C L, Monni O, Wagner U, Kononen J, Barlund M, Bucher C, Haas P, Nocito A, Bissig H, Sauter G, Kallioniemi A: High-throughput copy number analysis of 17q23 in 3520 tissue specimens by fluorescence in situ hybridization to tissue microarrays. Am J Pathol 2002, 161:73-79.
36. Hyman E, Kauraniemi P, Hautaniemi S, Wolf M, Mousses S, Rozenblum E, Ringner M, Sauter G, Monni O, Elkahloun A, Kallioniemi O P, Kallioniemi A: Impact of DNA amplification on gene expression patterns in breast cancer. Cancer Res 2002, 62:6240-6245.
37. Platzer P, Upender M B, Wislon K, Willis J, Lutterbaugh J, Nosrati A, Willson J K V, mack D, Ried T, Markowitz S: Silence of chromosomal amplifications in colon cancer. Cancer Res 2002, 62:1134-1138.
38. Patient R K, McGhee J D. The GATA family (vertebrates and invertebrates). Curr Opin Genet Dev 2002, 12:416-422.
39. Kuo C T, Morrisey E E, Anandappa R, Sigrist K, Lu M M, Parmacek M S, Soudais C, Leiden J M. GATA4 transcription factor is required for ventral morphogenesis and heart tube formation. Genes Dev 1997, 11:1048-1060.
40. Molkentin J D, Lin Q, Duncan S A, Olson E N. Requirement of the transcription factor GATA4 for heart tube formation and ventral morphogenesis. Genes Dev 1997, 11:1061-1072.
41. Lee K F, Simon H, Chen H, Bates B, Hung M C, Hauser C. Requirement for neuregulin receptor erbB2 in neural and cardiac development. Nature 1995, 378:394-398.
42. Garratt A N, Ozcelik C, Birchmeier C: ErbB2 pathways in heart and neural diseases. Trends Cardiovasc Med 2003, 13:80-86.
43. Han J, Lee J D, Jiang Y, Li Z, Feng L, Ulevitch R J: Characterization of the structure and function of a novel MAP kinase kinase (MKK6). J Biol Chem 1996, 271:2886-2891.
44. Schneider J W, Chang A Y, Garratt A. Trastuzumab cardiotoxicity: Speculations regarding pathophysiology and targets for further study. Semin Oncol 2002, 29:22-28.
45. Charron F, Tsimiklis G, Arcand M, Robitaille L, Liang Q, Molkentin J D, Meloche S, Nemer M: Tissue-specific GATA factors are transcriptional effectors of the small GTPase RhoA. Genes Dev 2001, 15:2702-2719.
46. Yanazume T, Hasegawa K, Wada H, Morimoto T, Abe M, Kawamura T, Sasayama S: Rho/ROCK pathway contributes to the activation of extracellular signal-regulated kinase/GATA4 during myocardial cell hypertrophy. J Biol Chem 2002, 277:8618-2865.
47. Arthur W T, Noren N K, Burridge K: Regulation of Rho family GTPases by cell-cell and cell-matrix adhesion. Biol Res 2002, 35:239-246.
48. Korsching E, Packeisen J, Agelopoulos K, Eisenacher M, Voss R, Isola J, van Diest P J, Brandt B, Boecker W, Buerger H: Cytogenetic alterations and cytokeratin expression patterns in breast cancer: integrating a new model of breast differentiation into cytogenetic pathways of breast carcinogenesis. Lab Invest 2002, 82:1525-1533.
49. Callagy G, Cattaneo E, Daigo Y, Happerfield L, Bobrow L G, Pharoah P D, Caldas C: Molecular classification of breast carcinomas using tissue microarrays. Diagn Mol Pathol 2003, 12:27-34.
50. Berns E M, Klijn J G; van Staveren I L, Portengen H, Noordegraaf E, Foekens J A: Prevalence of amplification of the oncogenes c-myc, HER2/neu, and int-2 in one thousand human breast tumors: correlation with steroid receptors. Eur J Cancer 1992, 28:697-700.
51. Keshgegian A A: ErbB-2 oncoprotein over-expression in breast carcinoma: inverse correlation with biochemically- and immunohistochemically-determined hormone receptors. Breast Cancer Res Treat 1995, 35:201-210.
52. Carlomagno C, Perrone F, Gallo C, De Laurentiis M, Lauria R, Morabito A, Pettinato G, Panico, L, D'Antonio A, Bianco A R, De Placido S: c-erb B2 over-expression decreases the benefit of adjuvant tamoxifen in early-stage breast cancer without axillary lymph node metastases. J Clin Oncol 1996, 14:2702-2708.
53. Konecny G, Pauletti G, Pegram M, Untch M, Dandekar S, Aguilar Z, Wilson C, Rong H M, Bauerfeind I, Felber M, Wang H J, Beryt M, Seshadri R, Hepp H, Slamon D J: Quantitative association between HER-2/neu and steroid hormone receptors in hormone receptor-positive primary breast cancer. J Natl Cancer Inst 2003, 95:142-153.
54. Pietras R J, Arboleda J, Reese D M, Wongvipat N, Pegram M D, Ramos L, Gorman C M, Parker M G, Sliwkowski M X, Slamon D J: HER-2 tyrosine kinase pathway targets estrogen receptor and promotes hormone-independent growth in human breast cancer cells. Oncogene 1995, 10:2435-2446.
55. Yang R B, Ng C K, Wasserman S M, Colman S D, Shenoy S, Mehraban F, Komuves L G, Tomlinson J E, Topper J N: Identification of a novel family of cell-surface proteins expressed in human vascular endothelium. J Biol Chem 2002, 277:46364-46373.
56. Willis S, Hutchins A M, Hammet F, Ciciulla J, Soo W K, White D, van der Spek P, Henderson M A, Gish K, Venter D J, Armes J E: Detailed gene copy number and RNA expression analysis of the 17q12-23 region in primary breast cancers. Genes Chromosomes Cancer 2003, 36:382-392
57. Dressman M A, Baras A, Malinowski R, Alvis L B, Kwon I, Walz T M, Polymeropoulos M H: Gene expression profiling detects gene amplification and differentiates tumor types in breast cancer. Cancer Res 2003, 63:2194-2199
58. Tan M, Yao J, Yu D: Over-expression of the c-erbB-2 gene enhanced intrinsic metastasis potential in human breast cancer cells without increasing their transformation abilities. Cancer Res 1997, 57:1199-1205.
59. Spencer K S, Graus-Porta D, Leng J, Hynes N E, Klemke R L: ErbB2 is necessary for induction of carcinoma cell invasion by ErbB family receptor tyrosine kinases. J Cell Biol 2000, 148:385-397.
60. Mackay A, Jones C, Dexter T, la Silva R, Bulmer K, Jones A, Simpson P, Harris R A, Jat P S, Neville A M, Reis L F L, Lakhani S R, O'Hare M J: cDNA microarray analysis of genes associated with ERBB2 (HER2/neu) over-expression in humna mammary luminal epithelial cells. Oncogene 2003, 22:2680-2688
61. Tomasetto C, Regnier C, Moog-Lutz C, Mattei M G, Chenard M P, Lidereau R, Basset P, Rio M C: Identification of four novel human genes amplified and over-expressed in breast carcinoma and localized to the q11-q21.3 region of chromosome 17. Genomics 1995, 28:3.67-376.
62. Kumar-Sinba C, Woods Ignatoski K, Lippman M E, Ethier S P, Chinnaiyan A M: Transcriptome analysis of HER2 reveals a molecular connection to fatty acid synthesis. Cancer Res 2003, 63: 132-139.
63. Tiwari R K, Mukhopadhyay B, Telang N T, Osborne M P: Modulation of gene expression by selected fatty acids in human breast cancer cells. Anticancer Res 1991, 11:1383-1388.
64. Gilde A J, Van Bilsen M: Peroxisome proliferator-activated receptors (PPARS): regulators of gene expression in heart and skeletal muscle. Acta Physiol Scand 2003, 178:425-434.
65. Press M F, Slamon D J, Flom I J, Park J, Zhou J Y, Bernstein L: Evaluation of HER-2/neu gene amplification and over-expression: comparison of frequently used assay methods in a molecularly characterized cohort of breast cancer specimens. J Clin Oncol 2002, 20:3095-3105.
66. van de Vijver M: Emerging technologies for HER2 testing. Oncology 2002, 63 Suppl 1:33-38.
67. Tagliabuea E, Agrestib R, Carcangiuc M L, Ghirellia C, Morellid D, Campiglioa M, Martelc M, Giovanazzib R, Grecob M, Balsarie A and Menard S: Role of HER2 in wound-induced breast carcinoma proliferation The Lancet Volume 362, Issue 9383, Pages 527-533

All documents referred to above are herein incorporated by reference in their entirety. A variety of modifications to the embodiments described will be apparent to those skilled in the art from the disclosure provided herein. Thus, the disclosure may be embodied in other specific forms without departing from the spirit or essential attributes thereof and, accordingly, reference should be made to the appended claims, rather than to the foregoing specification, as indicating the scope of the disclosure.

1. A method for identifying ERBB2 alteration in human breast tumors based on analysis of over-expression or under-expression of polynucleotide sequences in a breast tissue sample or tumor cell line from a patient comprising detecting the over-expression or under-expression of at least one polynucleotide sequence, or complement thereof, selected from each of the following predefined polynucleotide sequences sets consisting of:
Set 1: SEQ ID NO. 73, 74, 75, 76, 77 (ERBB2);
Set 2: SEQ ID NO. 28, 29, 30 (GRB7);
Set 3: SEQ ID NO. 83, 84, 85 (NR1D1);
Set 4: SEQ ID NO. 78, 79, 80 (GATA4);
Set 5: SEQ ID NO. 41, 42, 43 (CDH15);
Set 6: SEQ ID NO. 16, 17 (LTA);
Set 7: SEQ ID NO. 86, 87, 116 (MAP2K6);
Set 8: SEQ ID NO. 54, 55, 113 (PECAM1);
Set 9: SEQ ID NO. 44, 45 (PPARBP);
Set 13: SEQ ID NO. 10 (LOC148696);
Set 18: SEQ ID NO. 24, 25 (STAT3);
Set 20: SEQ ID NO. 36, 37, 38 (CDKL5);
Set 21: SEQ ID NO. 46, 47, 48 (CSTA);
Set 22: SEQ ID NO. 52, 53, 115 (ITGB3);
Set 23: SEQ ID NO. 56, 57, 58 (MKI67);
Set 24: SEQ ID NO. 59, 60, 61 (PBEF);
Set 27: SEQ ID NO. 88, 89, 90 (ITGA2);
Set 28: SEQ ID NO. 11 (ESTAA878915);
Set 29: SEQ ID NO. 1, 2, 3 (JDP1);
Set 35: SEQ ID NO. 67, 68, 69 (FLJ10193);
Set 36: SEQ ID NO. 70, 71, 72 (ESR1);
Set 43: SEQ ID NO. 104, 105, 106 (DAXX);
Set 47: SEQ ID NO. 114; and
Set 48: SEQ ID NO. 117, 118 (C17ORF37),
and outputting the over-expression or under-expression in a user readable format to identify ERBB2 alteration.

2. A method for identifying ERBB2 alteration in human breast tumors based on analysis of over-expression or under-expression of polynucleotide sequences in a breast tissue sample or tumor cell line from a patient comprising detecting the over-expression or under-expression of at least one polynucleotide sequence, or complement thereof, selected from each of the following predefined polynucleotide sequences sets consisting of:
Set 1: SEQ ID NO. 73, 74, 75, 76, 77 (ERBB2);
Set 2: SEQ ID NO. 28, 29, 30 (GRB7);
Set 3: SEQ ID NO. 83, 84, 85 (NR1D1);
Set 4: SEQ ID NO. 78, 79, 80 (GATA4);
Set 5: SEQ ID NO. 41, 42, 43 (CDH15);
Set 6: SEQ ID NO. 16, 17 (LTA);
Set 7: SEQ ID NO. 86, 87, 116 (MAP2K6);
Set 8: SEQ ID NO. 54, 55, 113 (PECAM1);
Set 9: SEQ ID NO. 44, 45 (PPARBP);
Set 10: SEQ ID NO. 33, 34, 35 (PPP1R1B);
Set 11: SEQ ID NO. 39, 40 (RPL19);
Set 12: SEQ ID NO. 4, 5, 6 (PSMB3);
Set 13: SEQ ID NO. 10 (LOC148696);
Set 14: SEQ ID NO. 12, 13 (NOL3/loc283849);
Set 15: SEQ ID NO. 14, 15 (ITGA2B);
Set 16: SEQ ID NO. 18, 19 (NFKBIE);
Set 17: SEQ ID NO. 22, 23 (PADI2);
Set 18: SEQ ID NO. 24, 25 (STAT3);
Set 19: SEQ ID NO. 26, 27 (OAS2);
Set 20: SEQ ID NO. 36, 37, 38 (CDKL5);
Set 21: SEQ ID NO. 46, 47, 48 (CSTA);
Set 22: SEQ ID NO. 52, 53, 115 (ITGB3);
Set 23: SEQ ID NO. 56, 57, 58 (MKI67);
Set 24: SEQ ID NO. 59, 60, 61 (PBEF);
Set 25: SEQ ID NO. 62, 63, 64 (FADS2);
Set 26: SEQ ID NO. 81, 82 (LOX);
Set 27: SEQ ID NO. 88, 89, 90 (ITGA2);
Set 28: SEQ ID NO. 11 (ESTAA878915/NA);
Set 29: SEQ ID NO. 1, 2, 3 (JDP1);
Set 30: SEQ ID NO. 7, 8, 9 (NAT1);
Set 31: SEQ ID NO. 20, 21 (CELSR2);
Set 32: SEQ ID NO. 31, 32 (ESTN33243/NA);
Set 33: SEQ ID NO. 49, 50, 51 (SCUBE2);
Set 34: SEQ ID NO. 65, 66 (ESTH29301/NA);
Set 35: SEQ ID NO. 67, 68, 69 (FLJ10193); and
Set 36: SEQ ID NO. 70, 71, 72 (ESR1),
and outputting the over-expression or under-expression in a user readable format to identify ERBB2 alteration.

3. The method according to claim 1 or 2, wherein the analysis consists of detecting the over-expression or the under-expression of all polynucleotide sequences, or complements thereof, in each predefined sequence set.

4. The method according to claim 1 or 2, wherein said detection of over-expression or under-expression of polynucleotide sequences is performed on polynucleotide sequences from the breast tissue sample.

5. The method according to claim 1 or 2, wherein said detection of over-expression or under-expression of polynucleotide sequences is performed on at least one polynucleotide array.

6. The method according to claim 1 or 2, wherein said detection of over-expression or under-expression of polynucleotide sequences is performed by measuring the level of a protein encoded by at least one of the polynucleotide sequences selected from each predetermined set.

7. The method according to claim 1 or 2, wherein said patient expresses a 2+ level of the HER2 protein on a 0 to 3+ scale in an immunohistochemical assay that determines over-expression of the HER2 protein by measuring binding of a polyclonal antibody to the HER2 protein.

8. The method according to claim 1 or 2, further comprising determining clinical efficacy of treatment of the patient with trastuzumab.

9. The method according to claim 1 or 2, wherein breast cancer is detected, diagnosed, staged, monitored, predicted, prevented or treated.

10. The method according to claim 9, wherein the stage or aggressiveness of the breast cancer is monitored.

11. A method for analyzing differential gene expression associated with a breast tumor based on the analysis of the over-expression or under-expression of polynucleotide sequences in a breast tissue sample or tumor cell line, said analysis comprising the detection of the over-expression or under-expression of at least one polynucleotide sequence, or complement thereof, selected from each of the following predefined polynucleotide sequences sets consisting of:
Set 1: SEQ ID NO. 73, 74, 75, 76, 77 (ERBB2);
Set 2: SEQ ID NO. 28, 29, 30 (GRB7);
Set 3: SEQ ID NO. 83, 84, 85 (NR1D1);
Set 4: SEQ ID NO. 78, 79, 80 (GATA4);
Set 5: SEQ ID NO. 41, 42, 43 (CDH15), and
and outputting the over-expression or under-expression in a user readable format to identify ERBB2 alteration.

12. The method of claim 11, further comprising detection of the over-expression of at least one polynucleotide sequence, or complement thereof, selected from each of the following predefined polynucleotide sequences sets consisting of:
Set 6: SEQ ID NO. 16, 17 (LTA);
Set 7: SEQ ID NO. 86, 87, 116 (MAP2K6); and
Set 8: SEQ ID NO. 54, 55, 113 (PECAM1).

13. The method of claim 11, further comprising detection of the over-expression of at least one polynucleotide sequence, or complement thereof, selected from each of the following predefined polynucleotide sequences sets consisting of:
Set 9: SEQ ID NO. 44, 45 (PPARBP);
Set 10: SEQ ID NO. 33, 34, 35 (PPP1R1B); and
Set 11: SEQ ID NO. 39, 40 (RPL19).

14. The method of claim 11, further comprising detection of the over-expression of at least one polynucleotide sequence, or complement thereof, selected from each of the following predefined polynucleotide sequences sets consisting of:
Set 6: SEQ ID NO. 16, 17 (LTA);
Set 7: SEQ ID NO. 86, 87, 116 (MAP2K6);
Set 8: SEQ ID NO. 54, 55, 113 (PECAM1);
Set 9: SEQ ID NO. 44, 45 (PPARBP);
Set 10: SEQ ID NO. 33, 34, 35 (PPP1R1B); and
Set 11: SEQ ID NO. 39, 40 (RPL19).

15. A method according to claim 11, 12, 13, or 14 wherein said differential gene expression corresponds to an alteration of ERBB2 gene expression in breast tumor.

16. A method according to claim 11, 12, 13, or 14 wherein said differential gene expression corresponds to an alteration of ER gene expression in breast tumor.

17. A method according to claim 11, 12, 13, or 14 wherein said detection of over-expression or under-expression of polynucleotide sequences is carried out by fluorescence in situ hybridization or immunohistochemistry.

18. A method according to claim 11, 12, 13, or 14 wherein said detection of over-expression or under-expression of polynucleotide sequences is performed on polynucleotides from a breast tissue, sample.

19. A method according to claim 11, 12, 13, or 14 wherein said detection of over-expression or under-expression of polynucleotide sequences is performed on polynucleotide sequences from a tumor cell line.

20. A method according to claim 11, 12, 13, or 14 wherein said detection of over-expression or under-expression of polynucleotide sequences is performed on at least one polynucleotide array.

21. A method according to claim 11, 12, 13, or 14 wherein said detection of over-expression or under-expression of polynucleotide sequences is performed by measuring the level of a protein encoded by at least one of the polynucleotide sequences selected from each predetermined set.

22. A method according to claim 21, wherein said detection is performed on proteins expressed from polynucleotides from a breast tissue sample or a tumor cell line.

23. A method according to claim 11, 12, 13, or 14 wherein said patient expresses a 2+ level of the HER2 protein on a 0 to 3+ scale in an immunohistochemical assay that determines over-expression of the HER2 protein by measuring binding of a polyclonal antibody to the HER2 protein.


SEQUENCE LISTING (118 sequences)

SEQ ID NO: 1 - DNA, 405 bp, Artificial Sequence
  Description of Artificial Sequence Synthetic DNA sequence
ttttaagcacaaattcattgcctttattctgatgagcttctcttcatgggatttacggacactgaattatatccagactctgctaacttcttggttttctccctcctatcgcctaatgactctttaagctactacatgagtctaatccatgggcatcctgagcttcacaaattcacgtcgcacccagcgagaaacccaccgtcttcactgagtcattcaaagcttcccactgctggaatggcatcgacatctggctccttcgccagtggtcatagcgggctcgactctcttcattggncagaatctcctttgccttctgcagtttctgaaaagtctccacagctttgggggttttnaggatgcttgtctggggtgacattccagagctctgactttaaattctgc

SEQ ID NO: 2 - DNA, 409 bp, Artificial Sequence
  Description of Artificial Sequence Synthetic DNA sequence
gattaagtcatctaaatggatgcaatactgaattacaggtcagaagatactgaagattactacacattactgggatgtgatgaactatcttcggttgaacaaatcctggcagaatttaaagtcagagctctggaatgtcacccagacaagcatcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtgaagacggtgggtttctcgctgggtgcgacgttgaatttgttgaagctcagggttgcccntgggtttaggactcatgttagttaggctttaaagngttctttag

SEQ ID NO: 3 - DNA, 1203 bp, Artificial Sequence
  Description of Artificial Sequence Synthetic DNA sequence
catctggtgtattgactgtggccagtcttaaagctagtttttgctatgtggaacatgctgctctaattcagatttaaagagtttcttcctgttaattcgaagctcactgtgcctcttgtttccgagggaagaaggactgattaagtcatctaaatggatgcaatactgaattacaggtcagaagatactgaagattactacacattactgggatgtgatgaactatcttcggttgaacaaatcctggcagaatttaaagtcagagctctggaatgtcacccagacaagcatcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtgaagacgtcaatgcactgggttgtcagaggtaaaaaagacctgatgctggaagaatctgacaagactcataccaccaagatggaaaatgaggaatgtaatgagcaaagagaaagaaagaaagaggagctggcttcaaccgcagagaaaacggagcagaaagaacccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgtggggagaaataaataattttcccaaagttcaaaaaaa

SEQ ID NO: 4 - DNA, 440 bp, Artificial Sequence
  Description of Artificial Sequence Synthetic DNA sequence
aattttgacaggctattttattncaaaaaaagaaaaaaaagtgggctctgggancagggttagnccattcgggcctncagtntcctgggggngattttgnccttctcgatgacntggacaatgactcccatgcctgacactgcatcccggnccacagcattcagcatggcttgggagatggtttcaaacaggtganccgganccatgttgggntcccagaggatctcacacattccgtacatttgtncggcgcaggtgccactgaccacaaagtcatcagtcaccatgggggcagccgatgaggtntaggagagcaaatgaagggnttaaaggtttttcgggntccaacccggcaatgactgggctcagtntaggtaagggggccaaaccnttttttcataacaggggtttggncaccatggcttcatgaggggtttaan

SEQ ID NO: 5 - DNA, 467 bp, Artificial Sequence
  Description of Artificial Sequence Synthetic DNA sequence
atccaggcccagttggtgaccacggacttccagaagatctttcccatgggtgaccggctgtacatcggtctggccgggctcgccactgacgtccagacagttgcccagcgcctcaagttccggctgaacctgtatgagttgaaggaaggtcggcagatcaaaccttataccctcatgagcatggtngccaacctcttgtatgagaaacggtttggcccttactacactgagccagtcattgccgggttggacccgaagacctttaagcccttcatttgctctctaggacctcatcggctgccccatgggtgactgatgactttgtgggtcagtgggcacctgcggccgaacaaatgttacggaatgtgtggagtcctttgggaggcccaacatgggttccggattcaactgttttgaaaaccattttcccaagccnggtggattgttttggaacngggtgnagtttt

SEQ ID NO: 6 - DNA, 784 bp, Artificial Sequence
  Description of Artificial Sequence Synthetic DNA sequence
gagcggttgcgcagtgaaggctagacccggtttactggaattgctctggcgatcgaggggtcctagtacaccgcaatcatgtctattatgtcctataacggaggggccgtcatggccatgaaggggaagaactgtgtggccatcgctgcagacaggcgcttcgggatccaggcccagatggtgaccacggacttccagaagatctttcccatgggtgaccggctgtacatcggtctggccgggctcgccactgacgtccagacagttgcccagcgcctcaagttccggctgaacctgtatgagttgaaggaaggtcggcagatcaaaccttataccctcatgagcatggtggccaacctcttgtatgagaaacggtttggcccttactacactgagccagtcattgccgggttggacccgaagacctttaagcccttcatttgctctctagacctcatcggctgccccatggtgactgatgactttgtggtcagtggcacctgcgccgaacaaatgtacggaatgtgtgagtccctctgggagcccaacatggatccggatcacctgtttgaaaccatctcccaagccatgctgaatgctgtggaccgggatgcagtgtcaggcatgggagtcattgtccacatcatcgagaaggacaaaatcaccaccaggacactgaaggcccgaatggactaaccctgttcccagagcccacttttttttctttttttgaaataaaatagcctgtctttcaaaaaaaaaaaaaaaaaaaaaaaaaaaaaaa

SEQ ID NO: 7 - DNA, 273 bp, Artificial Sequence
  Description of Artificial Sequence Synthetic DNA sequence
acaatattttatttactcatctaccaataaaacttttctaggaattcaacaataaaccaacattaaaagctttctagcataaatcaccaatttccaagataaccacaggccatctttaaaatacattttttattattattattattattatttgaaaaggtttgtggttatgtttctttaaaaagctgtttaattatatatgatgacatttttatagggtgaaatgatttgatgtctagggnttttcttcaaaataagggtaaggggtacagg

SEQ ID NO: 8 - DNA, 425 bp, Artificial Sequence
  Description of Artificial Sequence Synthetic DNA sequence
naagattttgagtctatgaatacatacctgcagacatctccatcatctgtgtttactagtaaatcattttgttccttgcagaccccagatgggttncactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtaaaacaatcttgtctatttgtcatccagctcaccagttatccaactgacggacctattcatgtatcnttctgtacccttacctttatttttggaaggaaaatcctaggacatccaaatccttttcacctattaaaaaat

SEQ ID NO: 9 - DNA, 1319 bp, Artificial Sequence
  Description of Artificial Sequence Synthetic DNA sequence
atacaatgaaagcactagaaataattattatacttataaccattgtatttttacatgtttaaaatatagccataattagcctactcaaatccaagtgtaaaagtaaaatgatttgctttcgttttgttttccttgcttaggggatcatggacattgaagcatatcttgaaagaattggctataagaagtctaggaacaaattggacttggaaacattaactgacattcttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaccaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaacccaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtgaaacaatcttgtctatttgtcatccagctcaccagttatcaactgacgacctatcatgtatcttctgtacccttaccttattttgaagaaatcctagacatcaaatcatttcacctataaaaatgtcatcatatataattaaacagctttttaaagaaacataaccacaaaccttttcaaataataataataataataataataaatgtcttttaaagaggcctgtggttatcttggaaattggtgatttatgctagaaagcttttaatgttggtttattgttgaattc

SEQ ID NO: 10 - DNA, 336 bp, Artificial Sequence
  Description of Artificial Sequence Synthetic DNA sequence
gggctcccaaaactttttatttagagggaagaatgctagggagatgggtatgcagagggttgaccaaattggaagaaaatatttattctgtagtttggtgttggaaaagggaattttccaatcagccacacctcagtgttgcggcaaaataattcttggctcccctggaaacgcatgggcaaggtagggcagagctgctgctgctgatactgccaccaccctgggcttcctgctgactctgggctactccctggggacaacagatttgcattgacgtccggggctgtccagaggccctcaagagccagttgtgagctgagcccagtatgggaaaga

SEQ ID NO: 11 - DNA, 356 bp, Artificial Sequence
  Description of Artificial Sequence Synthetic DNA sequence
tttgagaaatagggttttttattatatattcatttttccatatttctggaaaatgaaacagacatggagtatttcaaagctgggagagatcattatattggagttaccttagaaagagggggctgtttctgccaccacaacaaagaatgaaacaacacaaatatttacgttttcttcttgtcctcataatgcttttactgcactcgtctttcaacctttaacatctactgcatctgtgactgttgtttcagtaaaacacggaagcttgaatgtaagctcatttcattgcctgctgcacaacacacaaaacagcattttcttattcttgaaatatgatagcaacgtctccttccatg

SEQ ID NO: 12 - DNA, 369 bp, Artificial Sequence
  Description of Artificial Sequence Synthetic DNA sequence
cattgtcannnttgttttaattgtctggcttctctctggactgggagctcagtgaggattctgaccagtgacttacacaaaaggcgctctatacatattataatatattcgcttactaaatgantaaggactttccaactgcgtttctgagttttacagatgggaaaactaaggccaaaggataggagtggggttcacacagctaagtctgaagggaaactgggaagcagcaaccacctctgcacctcacctgggtctaaggagggggttcagggacttggggccaccaaactctaggggcctgtcccctcagggtgcaattncagctgctccnagggctatggccaaccctcttgccagagaggcagc

SEQ ID NO: 13 - DNA, 1411 bp, Artificial Sequence
  Description of Artificial Sequence Synthetic DNA sequence
ggcacgagggagaggcaggaggacaccgagttccccgtgttggcctccaggtcctgtgcttgcggagccgtccggcggctgggatcgagccccgacaatgggcaacgcgcaggagcggccgtcagagactatcgaccgcgagcggaaacgcctggtcgagacgctgcaggcggactcgggactgctgttggacgcgctgctggcgcggggcgtgctcaccgggccagagtacgaggcattggatgcactgcctgatgccgagcgcagggtgcgccgcctactgctgctggtgcagggcaagggcgaggccgcctgccaggagctgctacgctgtgcccagcgtaccgcgggcgcgccggaccccgcttgggactggcagcacgtgggtccgggctaccgggaccgcagctatgaccctccatgcccaggccactggacgccggaggcacccggctcggggaccacatgccccgggttgcccagagcttcagaccctgacgaggccgggggccctgagggctccgaggcggtgcaatccgggaccccggaggagccagagccagagctggaagctgaggcctctaaagaggctgaaccggagccggagccagagccagagctggaacccgaggctgaagcagaaccagagccggaactggagccagaaccggacccagagcccgagcccgacttcgaggaaagggacgagtccgaagattcctgaaggccagagctctgacaggcggtgccccgcccatgctggataggacctgggatgctgctggagctgaatcggatgccaccaaggctcggtccagcccagtaccgctggaagtgaataaactccggagggtcggacgggacctgggctctctccacgattctggctgtttgcccaggaacttagggtgggtacctctgagtcccagggacctgggcaggcccaagcccaccacgagcatcatccagtcctcagccctaatctgcccttaggagtccaggctgcaccctggagatcccaaacctagccccctagtgggacaaggacctgaccctcctgcccgcatacacaacccatttcccctggtgagccacttggcagcatatgtaggtaccagctcaaccccacgcaagttcctgagctgaacatggagcaaggggagggtgacttctctccacatagggagggcttagagctcacagccttgggaagtgagactagaagaggggagcagaaagggaccttgagtagacaaaggccacacacatcattgtcattactgttttaattgtctggcttctctctggactgggagctcagtgaggattctgaccagtgacttacacaaaaggcgctctatacatattataatatattcgcttactaaatgaaaaaaaaaaaaaaaaaaaa

SEQ ID NO: 14 - DNA, 545 bp, Artificial Sequence
  Description of Artificial Sequence Synthetic DNA sequence
gggggggtagcccagctctgttgggagggaaacgacaccaagaggacccaatggaacagctccaacccaaagcttggaggcaacttgttggagaaggggcggtgcaggtagcacgcccaaccctcctgctagaatagtgtaggctgcaccatcactccccctcttcatcatcttcttccaggggtggccggttccgcttgaagaagccgaccttccacatggccaggaccaggatggtgagcagcagcaggccacccagcacacccaccagcacccaccagattggaatggccctctcctccaaggcccggagcagctgtgtccacacctgagcttcccctcggggcaggctgagcgggggcaccgcataggggagggaggacacgttgaaccatgcgtgcgactgcagcacaaactgatccagaggcctctggtagaagctgggcaccacaaggaaggcagcaccgtgaacatggccgctgccgcgcgccatctcctgcaggtcacctgcaccaagtacaggcggcgggtcgcactttcgagaa

SEQ ID NO: 15 - DNA, 3334 bp, Artificial Sequence
  Description of Artificial Sequence Synthetic DNA sequence
attcctgcctgggaggttgtggaagaaggaagatggccagagctttgtgtccactgcaagccctctggcttctggagtgggtgctgctgctcttgggaccttgtgctgcccctccagcctgggccttgaacctggacccagtgcagctcaccttctatgcaggccccaatggcagccagtttggattttcactggacttccacaaggacagccatgggagagtggccatcgtggtgggcgccccgcggaccctgggccccagccaggaggagacgggcggcgtgttcctgtgcccctggagggccgagggcggccagtgcccctcgctgctctttgacctccgtgatgagacccgaaatgtaggctcccaaactttacaaaccttcaaggcccgccaaggactgggggcgtcggtcgtcagctggagcgacgtcattgtggcctgcgccccctggcagcactggaacgtcctagaaaagactgaggaggctgagaagacgcccgtaggtagctgctttttggctcagccagagagcggccgccgcgccgagtactccccctgtcgcgggaacaccctgagccgcatttacgtggaaaatgattttagctgggacaagcgttactgtgaagcgggcttcagctccgtggtcactcaggccggagagctggtgcttggggctcctggcggctattatttcttaggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcgcagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcagaccaagacacccgtgagctgcttcaacatccagatgtgtgttggagccactgggcacaacattcctcagaagctatccctaaatgccgagctgcagctggaccggcagaagccccgccagggccggcgggtgctgctgctgggctctcaacaggcaggcaccaccctgaacctggatctgggcggaaagcacagccccatctgccacaccaccatggccttccttcgagatgaggcagacttccgggacaagctgagccccattgtgctcagcctcaatgtgtccctaccgcccacggaggctggaatggcccctgctgtcgtgctgcatggagacacccatgtgcaggagcagacacgaatcgtcctggactctggggaagatgacgtatgtgtgccccagcttcagctcactgccagcgtgacgggctccccgctcctagttggggcagataatgtcctggagctgcagatggacgcagccaacgagggcgagggggcctatgaagcagagctggccgtgcacctgccccagggcgcccactacatgcgggccctaagcaatgtcgagggctttgagagactcatctgtaatcagaagaaggagaatgagaccagggtggtgctgtgtgagctgggcaaccccatgaagaagaacgcccagataggaatcgcgatgttggtgagcgtggggaatctggaagaggctggggagtctgtgtccttccagctgcagatacggagcaagaacagccagaatccaaacagcaagattgtgctgctggacgtgccggtccgggcagaggcccaagtggagctgcgagggaactcctttccagcctccctggtggtggcagcagaagaaggtgagagggagcagaacagcttggacagctggggacccaaagtggagcacacctatgagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtggactgggggctgcccatccccagcccctcccccattcacccggcccatcacaagcgggatcgcagacagatcttcctgccagagcccgagcagccctcgaggcttcaggatccagttctcgtaagctgcgactcggcgccctgtactgtggtgcagtgtgacctgcaggagatggcgcgcgggcagcgggccatggtcacggtgctggccttcctgtggctgcccagcctctaccagaggcctctggatcagtttgtgctgcagtcgcacgcatggttcaacgtgtcctccctcccctatgcggtgcccccgctcagcctgccccgaggggaagctcaggtgtggacacagctgctccgggccttggaggagagggccattccaatctggtgggtgctggtgggtgtgctgggtggcctgctgctgctcaccatcctggtcctggccatgtggaaggtcggcttcttcaagcggaaccggccacccctggaagaagatgatgaagagggggagtgatggtgcagcctacactattctagcaggagggttgggcgtgctacctgcaccgccccttctccaacaagttgcctccaagctttgggttggagctgttccattgggtcctcttggtgtcgtttccctcccaacagagctgggctaccccccctcctgctgcctaataaagagactgagccctg

SEQ ID NO: 16 - DNA, 639 bp, Artificial Sequence
  Description of Artificial Sequence Synthetic DNA sequence
ttttttttttcagagaaaagaggtttattgggcttcatcgagggtgcagatgcctccgtgtggggctctggtcggcagctggctttcagagcctttccctgccttctggggccctgtgatccctcatgcctacttctttctctcttggtcagccttgtgcgcatgccctctcactcttcatctcttgggcctgtctctgtttctccttggatgttcttctattattcccctctctccatcctccataaataaataatttaattttttttgccttcataaatagtcccctccctgcctctagtcatcccccaagctcctccatgtgcctgctcttcctctgtgtgtggatctaggccccacctagctggtgggacagaccaacagctttgggctgggaattcctaggcaggcttgaaatcctcagccagacagacatcagggatggttcagggaggtgtggtcccctggatgcctagaattccttctttgaaagctccggtgacttgatcaggggagacttgagctgttggaatggcaaagagaggtggtgacgaccctgcaatggtcagaatggaggcagaaatggggagaaggcttgaaatcattanttttctttctggattttccaagtctacagag

SEQ ID NO: 17 - DNA, 1386 bp, Artificial Sequence
  Description of Artificial Sequence Synthetic DNA sequence
gccccatctccttgggctgcccgtgcttcgtgctttggactaccgcccagcagtgtcctgccctctgcctgggcctcggtccctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggaccacacctccctgaaccatccctgatgtctgtctggctgaggatttcaagcctgcctaggaattcccagcccaaagctgttggtctgtcccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctg

SEQ ID NO: 18 - DNA, 255 bp, Artificial Sequence
  Description of Artificial Sequence Synthetic DNA sequence
ctagaagggaaaaactttttttaacgtctctgccattttagtgtcatgttactgaaacttcccttccccagtggctcagttcagagccttctgtgtgaagtctttaaacaacggtgtttcagggtcctcaacagcaataagatgggacacctctcagggttctgcttgctcctatttcagtctcttctcactcaggagagtccatgcttcctactggttggcagtttcaggctgacccaacatgggtaaaacaag

SEQ ID NO: 19 - DNA, 2067 bp, Artificial Sequence
  Description of Artificial Sequence Synthetic DNA sequence
ggaattccaatgaatgaatgaatgaatgagtgaatgaatcaacgaaggagtgagtcaaggcccgggaaccacagactccaagcctacgcagagcccgggaagggggattccggaggggcggggcctctttccggaagcgcccgccgggggcggggagggggcggggccatccgcgtgaggcgaccctgttggtccggaggggcggggcgaggaggaggacggccttgggcggttcggctgcccacagtaaccgctgggtggacctggccagcgctccgaaccttgtcctcgctgcgcgccggcccctcggagccccacagcccgggaaggaggcggccgcgggcggggcgcccgctctgccaagcggacccgcaacccggaaaggcggcgcggcggagcctggagccggatcctgctcagaccgggccccggccggccagagccgcgggcatgtcggaggcgcggaaggggccggacgaggcggaggagagccagtacgactctggcattgagtctctgcgctctctgcgctccctacccgagtccacctcggctccagcctccgggccctcggacggcagcccccagccctgcacccatcctccgggacccgtcaaggaaccacaggagaaggaagacgcggatggggagcgggctgattccacctatggctcctcctcgctcacctacaccctgtccttgctggggggccccgaggctgaggacccgcccccacgcctgccactcccccacgtgggggcgctgagccctcagcagctggaagcactcacttacatctccgaggacggagacacgctggtccacctggcagtgattcatgaggccccagcggtgctgctctgttgcctggctttgctgccccaggaggtcctggacattcaaaataacctttaccagacagcactccatctggctgtacatctggaccaaccgggcgcagttcgggcactggtgctgaagggggccagccgggcactacaggaccggcatggagacacagcccttcatgtggcctgccagcgccagtcttggcctgtgcccgctgcctgctggaagggcgggccagagccaggcagaggaacatctcactctctggacctccagctgcaaaactggcaaggtctggcttgtctccacattgccacccttcagaagaaccaaccactcatggaattgctgcttcggaatggagctgacattgatgtgcaggagggctccagtggtaagacagcgctgcacctggctgtggaaacccaagagcggggcctggtacagttcctgctccaggctggtgcccaggtagatgcccgcatgctgaacgggtgcacacccctgcacctggcagctggccggggtctcatgggcatctcatccactctgtgcaaggcgggtgctgactccctgctgcggaatgtggaggatgagacgccccaggacctgactgaggaatcccttgtccttttgccctttgatgacctgaagatctcagggaaactgctgctgtgtaccgactgaagccaggcagggtctgggatcctcagggctccacctctccatctggaagccggagccataactgctgcagtttgggcccaggctatgtgctcttctggtgccctagggactgctgtggccagagcctggggccagccagtacagtcctgagccgaggaggagggactgcaagtggaagagagccagtctggaaggaagagctttccaggtggacagggcttcttggaagacccccaaagccccaggtatcctgggtgaagcctgtttgcctctcttgaaaatggcaggtgctcttgttttacccatgttgggtcagcctgaaactgccaaccagtaggaagcatggactctcctgagtgagaagagactgaaataggagcaagcagaaccctgagaggtgtccatcttcttgctgttgaggaccctgaaacaccgttgtttaaagacttcacacagaaggctctgaactgagccactggggaagggaagtttcagtaacatgacactaaaatggcagagacgttaaaaaaa

SEQ ID NO: 20 - DNA, 498 bp, Artificial Sequence
  Description of Artificial Sequence Synthetic DNA sequence
aagtcatgattaatttnaaaccagttaaatttttaaccctttgcaggaattgctgaggggagaagacagggggagaatccacggcagaaaagccagagntgggcctcacaaatgagaaacaggagcctttctttcctgcccacagcctctttttctagccacctgctccagaaggaaagtcagtgacaagtctggaatacatgcttggagtaaatggtgactcagatgaaaagcagtcggcaaaactgaggagaggggaggagaggtggggagatgatggcctggggcaaggggaagggcgtcaagccccaagccagggctgctgggaacacccagcctgtgatggccatatcagacccccgggactggacacgaacccatcccaacgcaaaaagcaaataaataacaaattttttaacttttttcttcacaggggagctggggttggantcggggagaaagggggacagggcctggnccttttgaaggagagagcca

SEQ ID NO: 21 - DNA, 10531 bp, Artificial Sequence
  Description of Artificial Sequence Synthetic DNA sequence
taggagccggaggaggagccgccgccgccgttgacccggccgccggccgggagctgggagagatgcggagcccggccaccggcgtccccctcccaacgccgccgccgccgctgctgctgctgttgctgctgctgctgccgccgccactattgggagaccaagtggggccctgtcgttccttggggtccaggggacgaggctcttcgggggcctgcgcccccatgggctggctctgtccatcctcagcgtcgaacctctggctctacaccagccgctgcagggatgcgggcactgagctgactggccacctggtaccccaccacgatggcctgagggtttggtgtccagaatccgaggcccatattcccctaccaccagctcctgaaggctgcccctggagctgtcgcctcctgggcattggaggccacctttccccacagggcaagctcacactgcccgaggagcacccgtgcttaaaggctccacggctcagatgccagtcctgcaagctggcacaggcccccgggctcagggcaggggaaaggtcaccagaagagtccctgggtgggcgtcggaaaaggaatgtaaatacagccccccagttccagccccccagctaccaggccacagtgccggagaaccagccagcaggcacccctgttgcatccctgagggccatcgacccggacgagggtgaggcaggtcgactggagtacaccatggatgccctctttgatagccgctccaaccagttcttctccctggacccagtcactggtgcagtaaccacagccgaggagctggatcgtgagaccaagagcacccacgtcttcagggtcacggcgcaggaccacggcatgccccgacgaagtgccctggctacactcaccatcttggttactgacaccaatgaccatgaccctgtgttcgagcagcaggagtacaaggagagcctcagggagaacctggaggttggctatgaggtgctcactgtcagggccacggatggtgatgcccctcccaatgccaatattctgtaccgcctgctggaggggtctgggggcagcccctctgaagtctttgagatcgaccctcgctctggggtgatccgaacccgtggccctgtggatcgggaagaggtggaatcctaccagctgacggtagaggcaagtgaccagggtcgggacccgggtcctcggagtaccacagccgctgttttcctttctgtggaggatgacaatgataatgccccccagtttagtgagaagcgctatgtggtccaggtgagggaggatgtgactccaggggccccagtactccgagtcacagcctcggatcgagacaaggggagcaatgccgtggtgcactatagcatcatgagtggcaatgctcggggacagttttatctggatgcccagactggagctctggatgtggtgagccctcttgactatgagacgaccaaggagtacaccctacgggtgcgagcacaggatggtggccgtcccccactctctaatgtctctggcttggtgacagtacaggtcctggatatcaacgacaatgcccccatcttcgtcagcacccctttccaggctactgtcctggagagcgtccccttaggctacctggttctccatgtccaggctatcgacgctgatgctggtgacaatgcccgcctggaataccgccttgctggggtgggacatgacttccccttcaccatcaacaatggcacaggctggatctctgtggctgctgaactggaccgggaggaagttgatttctacagctttggggtagaagctcgagaccatggcactccagcactcactgcctcggccagtgtcagcgtgactgtcctggatgtcaacgacaacaatccaacctttacccaaccagagtacacagtgcggctcaatgaggatgcagctgtgggcaccagcgtggtgacggtgtcagctgtggaccgtgatgctcatagtgtcatcacctaccagatcaccagtggcaatactcgaaaccgcttctccatcaccagccaaagtggtggtgggctggtatcccttgccctgccactggactacaaacttgagcggcagtatgtgttggctgttaccgcctccgatggcactcggcaggacacggcacagattgtggtgaatgtcaccgacgccaacacccatcgtcctgtctttcagagctcccactatacagtgaatgttaatgaggaccggccggcaggcaccacggtggtgctgatcagcgccacggatgaggacacaggtgagaatgcccgcatcacctacttcatggaggacagcatcccccagttccgcatcgatgcagacacgggggctgtcaccacccaggctgagctggactacgaagaccaagtgtcttacaccctggccattactgctcgggacaatggcattccccagaagtccgacaccacctacctggagatcctggtgaacgacgtgaatgacaatgcccctcagttcctgcgagactcctaccagggcagtgtctatgaggatgtgccacccttcactagcgtcctgcagatctcagccactgatcgtgattctggacttaatggcagggtcttctacaccttccaaggaggcgacgatggagacggtgactttattgttgagtccacgtcaggcatcgtgcgaacgctacggaggctggatcgagagaacgtggcccagtatgtcttgcgggcatatgcagtggacaaggggatgcccccagcccgcacacctatggaagtgacagtcactgtgttggatgtgaatgacaatccccctgtctttgagcaggatgagtttgatgtgtttgtggaagagaacagccccattgggctagccgtggcccgggtcacagccactgaccccgatgaaggcaccaatgcccagattatgtaccagattgtggagggcaacatccctgaggtcttccagctggacatcttctccggggagctgacagccctggtagacttagactacgaggaccggcctgagtacgtcctggtcatccaggccacgtcagctcctctggtgagccgggctacagtccacgtccgcctccttgaccgcaatgacaacccaccagtgctgggcaactttgagatccttttcaacaactatgtcaccaatcgctcaagcagcttccctgggggtgccattggccgagtacctgcccatgaccctgatatctcagatagtctgacttacagctttgagcggggaaatgaactcagcctggtcctgctcaatgcctccacgggtgagctgaagctaagccgcgcactggacaacaaccggcctctggaggccatcatgagcgtgctggtgtcagacggcgtacacagcgtgaccgcccagtgcgcgctgcgtgtgaccatcatcaccgatgagatgctcacccacagcatcacgctgcgcctggaggacatgtcacccgagcgcttcctgtcaccactgctaggcctcttcatccaggcggtggccgccacgctggccacgccaccggaccacgtggtggtcttcaacgtacagcgggacaccgacgcccccgggggccacatcctcaacgtgagcctgtcggtgggccagccgccagggcccgggggcgggccgcccttcctgccctctgaggacctgcaggagcgcctatacctcaaccgcagcctgctgacggccatctcggcacagcgcgtgctgcccttcgacgacaacatctgcctgcgggagccctgcgagaactacatgcgctgcgtgtcggtgctgcgcttcgactcctccgcgcccttcatcgcctcctcctccgtgctcttccggcccatccaccccgtcggagggctgcgctgccgctgcccgcccggcttcacgggtgactactgcgagaccgaggtggacctctgctactcgcggccctgtggcccccacgggcgctgccgcagccgcgagggcggctacacctgcctctgtcgtgatggctacacgggtgagcactgtgaggtgagtgctcgctcaggccgttgcaccccgggtgtctgcaagaatgggggcacctgtgtcaacctgctggtgggcggtttcaagtgcgattgcccatctggagacttcgagaagccctactgccaggtgaccacgcgcagcttccccgcccactccttcatcacctttcgcggcctgcgccagcgtttccacttcaccctggccctctcgtttgccacaaaggagcgcgacgggttgctgttgtacaatgggcgtttcaatgagaagcatgactttgtggccctcgaggtgatccaggagcaggtccagctcaccttctctgcaggggagtcaaccaccacggtgtccccattcgtgcccggaggagtcagtgatggccagtggcatacggtgcagctgaaatactacaataagccactgttgggtcagacagggctcccacagggcccatcagagcagaaggtggctgtggtgaccgtggatggctgtgacacaggagtggccttgcgcttcggatctgtcctgggcaactactcctgtgctgcccagggcacccagggtggcagcaagaagtctctggatctgacggggcccctgctactaggcggggtgcctgacctgcccgagagcttcccagtccgaatgcggcagttcgtgggctgcatgcggaacctgcaggtggacagccggcacatagacatggctgacttcattgccaacaatggcaccgtgcctggctgccctgccaagaagaacgtgtgtgacagcaacacttgccacaatgggggcacttgcgtgaaccagtgggacgcgttcagctgcgagtgccccctgggctttgggggcaagagctgcgcccaggaaatggccaatccacagcacttcctgggcagcagcctggtggcctggcatggcctctcgctgcccatctcccaaccctggtacctcagcctcatgttccgcacgcgccaggccgacggtgtcctgctgcaggccatcaccagggggcgcagcaccatcaccctacagctacgagagggccacgtgatgctgagcgtggagggcacagggcttcaggcctcctctctccgtctggagccaggccgggccaatgacggtgactggcaccatgcacagctggcactgggagccagcggggggcctggccatgccattctgtccttcgattatgggcagcagagagcagagggcaacctgggcccccggctgcatggtctgcacctgagcaacataacagtgggcggaatacctgggccagccggcggtgtggcccgtggctttcggggctgtttgcagggtgtgcgggtgagcgatacgccagagggggttaacagcctggatcccagccatggggagagcatcaacgtggagcaaggctgtagcctgcctgacccttgtgactcaaacccgtgtcctgctaacagctattgcagcaacgactgggacagctattcctgcagctgtgatccaggttactatggtgacaactgtactaatgtgtgtgacctgaacccgtgtgagcaccagtctgtgtgtacccgcaagcccagtgccccccatggctatacctgcgagtgtcccccaaattaccttgggccatactgtgagaccaggattgaccagccttgtccccgtggctggtggggacatcccacatgtggcccatgcaactgtgatgtcagcaaaggctttgacccagactgcaacaagacaagcggcgagtgccactgcaaggagaaccactaccggcccccaggcagccccacctgcctcttgtgtgactgctaccccacaggctccttgtccagagtctgtgaccctgaggatggccagtgtccatgcaagccaggtgtcatcgggcgtcagtgtgaccgctgtgacaacccttttgctgaggtcaccaccaatggctgtgaagtgaattatgacagctgcccacgagcgattgaggctgggatctggtggccccgtacccgcttcgggctgcctgctgctgctccctgtcccaaaggctcctttgggactgctgtgcgccactgtgatgagcacagggggtggctccccccaaacctcttcaactgcacgtccatcaccttctcagaactgaagggcttcgctgagcggctacagcggaatgagtcaggcctagactcagggcgctcccagcagctagccctgctcctgcgcaacgccacgcagcacacagctggctacttcggcagcgacgtcaaggtggcctaccagctggccacgcggctgctggcccacgagagcacccagcggggctttgggctgtctgccacacaggacgtgcacttcactgagaatctgctgcgggtgggcagcgccctcctggacacagccaacaagcggcactgggagctgatccagcagacagagggtggcaccgcctggctgctccagcactatgaggcctacgccagtgccctggcccagaacatgcggcacacctacctaagccccttcaccatcgtcacgcccaacattgtcatctccgtagtgcgcttggacaaagggaactttgctggggccaagctgccccgctacgaggccctgcgtggggagcagcccccggaccttgagacaacagtcattctgcctgagtctgtcttcagagagacgccccccgtggtcaggcccgcaggccccggagaggcccaggagccagaggagctggcacggcgacagcgacggcacccggagctgagccagggtgaggctgtggccagcgtcatcatctaccgcaccctggccgggctactgcctcataactatgaccctgacaagcgcagcttgagagtccccaaacgcccgatcatcaacacacccgtggtgagcatcagcgtccatgatgatgaggagcttctgccccgggccctggacaaacccgtcacggtgcagttccgcctgctggagacagaggagcggaccaagcccatctgtgtcttctggaaccattcaatcctggtcagtggcacaggtggctggtcggccagaggctgtgaagtcgtcttccgcaatgagagccacgtcagctgccagtgcaaccacatgacgagcttcgctgtgctcatggacgtttctcggcgggagaatggggagatcctgccactgaagacactgacatacgtggctctaggtgtcaccttggctgcccttctgctcaccttcttcttcctcactctcttgcgtatcctgcgctccaaccaacacggcatccgacgtaacctgacagctgccctgggcctggctcagctggtcttcctcctgggaatcaaccaggctgacctcccttttgcctgcacagtcattgccatcctgctgcacttcctgtacctctgcaccttttcctgggctctgctggaggccttgcacctgtaccgggcactcactgaggtgcgcgatgtcaacaccggccccatgcgcttctactacatgctgggctggggcgtgcctgccttcatcacagggctagccgtgggcctggaccccgagggctacgggaaccctgacttctgctggctctccatctatgacacgctcatctggagttttgctggcccggtggcctttgccgtctcgatgagtgtcttcctgtacatcctggcggcccgggcctcctgtgctgcccagcggcagggctttgagaagaaaggtcctgtctcgggcctgcagccctccttcgccgtcctcctgctgctgagcgccacgtggctgctggcactgctctctgtcaacagcgacaccctcctcttccactacctctttgctacctgcaattgcatccagggccccttcatcttcctctcctatgtggtgcttagcaaggaggtccggaaagcactcaagcttgcctgcagccgcaagcccagccctgaccctgctctgaccaccaagtccaccctgacctcgtcctacaactgccccagcccctacgcagatgggcggctgtaccagccctacggagactcggccggctctctgcacagcaccagtcgctcgggcaagagtcagcccagctacatccccttcttgctgagggaggagtccgcactgaaccctggccaagggccccctggcctgggggatccaggcagcctgttcctggaaggtcaagaccagcagcatgatcctgacacggactccgacagtgacctgtccttagaagacgaccagagtggctcctatgcctctacccactcatcagacagtgaggaggaagaagaggaggaggaagaggaggccgccttccctggagagcagggctgggatagcctgctggggcctggagcagagagactgcccctgcacagtactcccaaggatgggggcccagggcctggcaaggccccctggccaggagactttgggaccacagcaaaagagagtagtggcaacggggcccctgaggagcggctgcgggagaatggagatgccctgtctcgagaggggtccctaggcccccttccaggctcttctgcccagcctcacaaaggcatccttaagaagaagtgtctgcccaccatcagcgagaagagcagcctcctgcggctccccctggagcaatgcacagggtcttcccggggctcctccgctagtgagggcagccggggcggcccccctccccgcccaccgccccggcagagcctccaggagcagctgaacggggtcatgcccatcgccatgagcatcaaggcaggcacggtggatgaggactcgtcaggctccgaatttctcttctttaacttcctgcattaaccctgggccgtggttcctacgcccgaggctcccttcccttccccagccgcactcatgccctgctcctgtcttgtgctttatcctgccccgctccccatcgcctgcccgcagcagcgacgaaacgtccatctgaggagcctgggccttgccgggaggggtactcaccccacctaaggccatctagtgccaactccccccccaccattcccctcactgcactttggacccctggggccaacatctccaagacaaagtttttcagaaaagaggaaaaaaagaatttaaaaaaggatctccactcttcatgacttcagggattcattttttttatacgctggaaattgactcccctttcccttcccaaagaggataggacctcccaggatgcttcccagcctctcctcagtttcccatctgctgtgcctctgggaggagagggactcctggggggcctgcccctcatacgccatcaccaaaaggaaaggacaaagccacacgcagccagggcttcacacccttcaggctgcacccgggcaggcctcagaacggtgaggggccagggcaaagggtgtgtctcgtcctgcccgcactgcctctcccaggaactggaaaagccctgtccggtgagggggcagaaggactcagcgcccctggacccccaaatgctgcatgaacacattttcaggggagcctgtgcccccaggcgggggtcgggcagccccagcccctctccttttcctggactctggccgtgcgcggcagcccaggtgtttgctcagttgctgacccaaaagtgcttcatttttcgtgcccgccccgcgccccgggcaggccagtcatgtgttaagttgcgcttctttgctgtgatgtgggtgggggaggaagagtaaacacagtgctggctcggctgccctgagggtgctcaatcaagcacaggtttcaagtctgggttctggtgtccactcacccaccccaccccccaaaatcagacaaatgctactttgtctaacctgctgtggcctctgagacatgttctatttttaaccccttcttggaattggctctcttcttcaaaggaccaggtcctgttcctctttctccccgactccaccccagctccctgtgaagagagagttaatatatttgttttatttatttgctttttgcgttgggatgggttcgtgtccagtcccgggggtctgatatggccatcacaggctgggtgttcccagcagccctggcttgggggcttgacgcccttccccttgccccaggccatcatctccccacctctcctcccctctcctcagttttgccgactgcttttcatctgagtcaccatttactccaagcatgtattccagacttgtcactgactttccttctggagcaggtggctagaaaaagaggctgtgggcaggaaagaaaggctcctgtttctcatttgtgaggccagcctctggcttttctgccgtggattctccccctgtcttctcccctcagcaattcctgcaaagggttaaaaatttaactggtttttactactgatgacttaaaaaaaatacaaagatgctggatgctaacttgatactaaccatcagattgtacagtttggttgttgctgtaaatatggtagcgttttgttgttgttgttttttcatgccccatactactgaataaactagttctgtgcgggt

SEQ ID NO: 22 - DNA, 459 bp, Artificial Sequence
  Description of Artificial Sequence Synthetic DNA sequence
ttgacttccttatgatnccatttatttcattgttctttagtcgagctcttccctaaacatctttagatctccaccacaggctcttttccagaaatttgaaactgtgttcttcttgccatcttcacgacatcccctgccctcttacataagatatttcaacatcaaggtggaagcaggaacttagctgagttttgcaacagagaagcgtattctaggcctacatttatagaaagtgggggtggggaagagccatgagtccacgggggtatatccacaccgagggttgtcacactgggtggggcaagtgagatggggaacgggtntgtgagtccnggggaacttcagaaacatcagaaattacccgacatcattggggaaagccttaggaaaaatctntaaaggcacacttgtctggcacatggggagggcgttcanttcccccnaattgtaggcttaaaa

SEQ ID NO: 23 - DNA, 2348 bp, Artificial Sequence
  Description of Artificial Sequence Synthetic DNA sequence
cgcacctgctgcaggtgctcccggccgccccggaccagcgagcgcgggcactgcggcggggaggatgctgcgcgagcggaccgtgcggctgcagtacgggagccgcgtggaggcggtgtacgtgctgggcacctacctctggaccgatgtctacagcgcggccccagccggggcccaaaccttcagcctgaagcactcggaacacgtgtgggtggaggtggtgcgtgatggggaggctgaggaggtggccaccaatggcaagcagcgctggcttctctcgcccagcaccaccctgcgggtcaccatgagccaggcgagcaccgaggccagcagtgacaaggtcaccgtcaactactatgacgaggaagggagcattcccatcgaccaggcggggctcttcctcacagccattgagatctccctggatgtggacgcagaccgggatggtgtggtggagaagaacaacccaaagaaggcatcctggacctggggccccgagggccagggggccatcctgctggtgaactgtgaccgagagacaccctggttgcccaaggaggactgccgtgatgagaaggtctacagcaaggaagatctcaaggacatgtcccagatgatcctgcggaccaaaggccccgaccgcctccccgccggatacgagatagttctgtacatttccatgtcagactcagacaaagtgggcgtgttctacgtggagaacccgttcttcggccaacgctatatccacatcctgggccggcggaagctctaccatgtggtcaagtacacgggtggctccgcggagctgctgttcttcgtggaaggcctctgtttccccgacgagggcttctcaggcctggtctccatccatgtcagcctgctggagtacatggcccaggacattcccctgactcccatcttcacggacaccgtgatattccggattgctccgtggatcatgacccccaacatcctgcctcccgtgtcggtgtttgtgtgctgcatgaaggataattacctgttcctgaaagaggtgaagaaccttgtggagaaaaccaactgtgagctgaaggtctgcttccagtacctaaaccgaggcgatcgctggatccaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcacccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctggactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggttgcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgt

SEQ ID NO: 24 - DNA, 600 bp, Artificial Sequence
  Description of Artificial Sequence Synthetic DNA sequence
tctcatttctctatttttaaaagcgcccagattgctcaaagatagcagaagtaggagattaaaaaaaatctggaaccacaaagttagtagtttcagatgatctggggtttggctgtgtgaggggtggcagaatgcaggtaggcgccttagtcgtatctttctgcagcttccgttctcagctcctcacatgggggaggtagcgcactccgaggtcaactccatgtcaaaggtgagggactcaaactgccctcctgctgagggttcagcaccttcaccattatttccaaactgcatcaatgaatctaaagtgcggggggacatcggcaggtcaatggtattgctgcaggtcgttggtgtcacacagataaacttggtcttcaggtatggggcagcgctacctgngtcagcttcaggatgctcctggctctctggccgacaatactttccgaatgcctcctccttgggaatgtcaggatagagatagaccagtggagacaccaggatattggtagcatccatgatcttatagcccatgatgatttcagcaaatgacatgttgttcagctgctgctttgtgtatggntccacggactggatctg

SEQ ID NO: 25 - DNA, 3455 bp, Artificial Sequence
  Description of Artificial Sequence Synthetic DNA sequence
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaaaaaaaaaaa

SEQ ID NO: 26 - DNA, 658 bp, Artificial Sequence
  Description of Artificial Sequence Synthetic DNA sequence
aaaagaagcaaaggaatggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagccccctaagcccccactacaagtgatcctcaggcaggtaaccccagattcatgcactgtagggtgctgcgcagcatccctagtctctacccagtagatgccactagccctcctctcccagtgacaaccaaaagtcttcagacattgtcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaaatgtcatgtggttacctgtaacttgaaggtggctacaaagatgactgtggacgtgggttgcact

SEQ ID NO: 27 - DNA, 3068 bp, Artificial Sequence
  Description of Artificial Sequence Synthetic DNA sequence
cggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtctgcaggaaccccgaacagttccccctggtgcagggagtggccataggtggctcctatggacggaaaacagtcttaagaggcaactccgatggtacccttgtccttttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggtccaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaacggttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgtcctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctcggcatcttctggaaggtcaattacaactttgaagatgagaccgtgaggaagtttctactgagccagttgcagaaaaccaggcctgtgatcttggacccaggcgaacccacaggtgacgtgggtggaggggaccgttggtgttggcatcttctggacaaagaagcaaaggttaggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagcccccctaagcccccactacaagtgatcctcaggcaggtaaccccagattcatgcactgtagggctgggcgcagcatccctaggtctctacccagtagatgccactagccctcctctcccagtgacaaccaaaagtcttcacatgttcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaatgtcatgtggttacctgtaacttgaaggtggctacaaagatgactgtggaggtggttgcacttgccacccaaggatgtctgccacacctctccaagccctcctacctaccaagatatacctgatatatccaccagatatctcctcagatatacttggttctctccaccaggttctttctttaaagcaggattctcaactttgatacttactcacattgggctagacagttctttgtttggaggctctcttgtgcatgtaggatgttgagcagcatgtgtggcctgtacccagtacatgccacccagttgtgacaattaaaagtgtcttgagactttatcatgtgtcttctgccctaggtgagaacccttgcactacaggaaccctacacccaacctggggggaatgtagggaagaggtgccaagccaaccgtggggttagctctaattattaagttatgcattataaataaataccaaaaaattg

SEQ ID NO: 28 - DNA, 286 bp, Artificial Sequence
  Description of Artificial Sequence Synthetic DNA sequence
aaaaaataatctttattgncactagtataaaacagagcagnncaactggcctntcggnctgtacaaagtgtggggcgtgaaaccgctgggctgcccccacttctcccanaattccctgccctagagcagcacctccagagctaggagaaggagagggggccacccaaggncttcccttgaggagaggggtcaggagtggactggagtgggggctnttttctatctgagggaggcaaagaagcagaggagaaaactggagtgggcggaaccctcccgccctcgtgcc

SEQ ID NO: 29 - DNA, 253 bp, Artificial Sequence
  Description of Artificial Sequence Synthetic DNA sequence
tgcctccctcagatagaaaacagcccccactccagtccactcctgacccctctcctcaagggaaggccttgggtggccccctctccttctcctagctctggaggtgctgctctagggcagggaattatgggagaagtnggggcagcccaggcgntttcacgcccacactttgtacagaccgagaggccagttgatctgctctgttttatactagtgacaataaagattattttttgatacaaaaaaaaaaaaa

SEQ ID NO: 30 - DNA, 2205 bp, Artificial Sequence
  Description of Artificial Sequence Synthetic DNA sequence
cacagggctcccccccgcctctgacttctctgtccgaagtcgggacaccctcctaccacctgtagagaagcgggagtggatctgaaataaaatccaggaatctgggggttcctagacggagccagacttcggaacgggtgtcctgctactcctgctggggctcctccaggacaagggcacacaactggttccgttaagcccctctctcgctcagacgccatggagctggatctgtctccacctcatcttagcagctctccggaagacctttggccagcccctgggacccctcctgggactccccggccccctgatacccctctgcctgaggaggtaaagaggtcccagcctctcctcatcccaaccaccggcaggaaacttcgagaggaggagaggcgtgccacctccctcccctctatccccaaccccttccctgagctctgcagtcctccctcacagagcccaattctcgggggcccctccagtgcaagggggctgctcccccgcgatgccagccgcccccatgtagtaaaggtgtacagtgaggatggggcctgcaggtctgtggaggtggcagcaggtgccacagctcgccacgtgtgtgaaatgctggtgcagcgagctcacgccttgagcgacgagacctgggggctggtggagtgccacccccacctagcactggagcggggtttggaggaccacgagtccgtggtggaagtgcaggctgcctggcccgtgggcggagatagccgcttcgtcttccggaaaaacttcgccaagtacgaactgttcaagagctccccacactccctgttcccagaaaaaatggtctccagctgtctcgatgcacacactggtatatcccatgaagacctcatccagaacttcctgaatgctggcagctttcctgagatccagggctttctgcagctgcggggttcaggacggaagctttggaaacgctttttctgtttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggacacaaggggcttcggatcttctgcagtgaagatgagcagagccgcacctgctggctggctgccttccgcctcttcaagtacggggtgcagctgtacaagaattaccagcaggcacagtctcgccatctgcatccatcttgtttgggctccccacccttgagaagtgcctcagataataccctggtggccatggacttctctggccatgctgggcgtgtcattgagaacccccgggaggctctgagtgtggccctggaggaggcccaggcctggaggaagaagacaaaccaccgcctcagcctgcccatgccagcctccggcacgagcctcagtgcagccatccaccgcacccaactctggttccacgggcgcatttcccgtgaggagagccagcggcttattggacagcagggcttggtagacggcctgttcctggtccgggagagtcagcggaacccccagggctttgtcctctctttgtgccacctgcagaaagtgaagcattatctcatcctgccgagcgaggaggagggtcgcctgtacttcagcatggatgatggccagacccgcttcactgacctgctgcagctcgtggagttccaccagctgaaccgcggcatcctgccgtgcttgctgcgccattgctgcacgcgggtggccctctgaccaggccgtggactggctcatgcctcagcccgccttcaggctgcccgccgcccctccacccatccagtggactctggggcgcggccacaggggacgggatgaggagcgggagggttccgccactccagttttctcctctgcttctttgcctccctcagatagaaaacagcccccactccagtccactcctgacccctctcctcaagggaaggccttgggtggccccctctccttctcctagctctggaggtgctgctctagggcagggaattatgggagaagtgggggcagcccaggcggtttcacgccccacactttgtacagaccgagaggccagttgatctgctctgttttatactagtgacaataaagattattttttgatac

SEQ ID NO: 31 - DNA, 380 bp, Artificial Sequence
  Description of Artificial Sequence Synthetic DNA sequence
tttttttttttggagcttggtaatgagcatttttattaaattccccaagtacaagccccggtgtacacaataagcaatttcagttttgagtaactgccagggaaggaatgtccccctttctgtttgttgactgccttctgatttgtttgaaagcttttttcttcttgcattatcttgaccattcctggtatgaaggatgttgtcttgtttctcctcagtttcatcttctgcagaagaagaaaccatgccaggcggagtctcttactgttcatggcttccatggctttacnctgttccctttttctctcacaagtcatcagctcagatctaaaaatactctaagggtaaatccactggngaataaaatggttccaatgtgc

SEQ ID NO: 32 - DNA, 300 bp, Artificial Sequence
  Description of Artificial Sequence Synthetic DNA sequence
aatactcagcaaatgtgaaactttatttgctcttacttcaaaattagtccaaaatgttggaaataaaatataagacattgatctagatatgaggtttttctccttcattctcagctgtcgaagaaatcaaagtagcatatgcacaaggttaaaaaccacatatacaaatactatagaacagcttataatgaaaaccttgcctgcctttataaaaaatgtgattatcttcttctgttaatgtcaataaaagatggtttgtcctagaaggtcttataagtggtaaatggtantatgntctgg

SEQ ID NO: 33 - DNA, 466 bp, Artificial Sequence
  Description of Artificial Sequence Synthetic DNA sequence
atttacaaagatttattacagcacgggaggggttcaggcctggagttagggaagaaggggaaaggggcagagcagctgggggacaaggaaaacctggcgccccccgctgtgtgccccaccgggacaataaactaggcggcattcctggcatcaaagcacaaaacgcaacaaagaggtctctgccagtccatcttccaggcacccaggaggagcaagggtgattaagggaagattcccaaaatgttgaggctatggagaaaaacgccttagtcctggaccctggtagaagccggtgagagaagtggtgacttggaatcctccataggaaagtgggtagaaaaggatctaagggtacctcaaggttctcaggacctcctttccccagatcttagggtcctgccctgtgggtctcctgtgtccaggggagaggatctggggagtagaattgtgaagggcaatcccgttc

SEQ ID NO: 34 - DNA, 352 bp, Artificial Sequence
  Description of Artificial Sequence Synthetic DNA sequence
ccacaggcnaggaccctaagatctggggaaaggaggtcctgagaaccttgaggtacccttagatccttttctacncactttcctatggaggattccaagtcaccacttctctcaccggcttctaccagggtccaggactaaggcgtttttctccatagcctcaacattttgggaatcttcccttaatcacccttgctcctcctgggtgcctggaagatggactggcagagacctctttgttgcgttttgtgctttggatgccaggaatgccgcctagtttatgtccccggtgggcacacagcgggggggcgccaaggttttccttggtcccccaagctggctctgcnccttt

SEQ ID NO: 35 - DNA, 1841 bp, Artificial Sequence
  Description of Artificial Sequence Synthetic DNA sequence
agctgggaccggagggtgagcccggcagaggcagagacacacgcggagaggaggagaggctgagggagggaggtggagaaggacgggagaggcagagagaggagacacgcagagacactcaggaggggagagacaccgagacgcagagacactcaggaggggagagacaccgagacgcagagacacccaggccggggagcgcgagggagcgaggcacagacctggctcagcgagcgcggggggcgagccccgagtcccgagagcctgggggcgcgcccagcccgggcgccgaccctcctcccgctcccgcgccctcccctcggcgggcacggtatttttatccgtgcgcgaacagccctcctcctcctctcgccgcacagcccgccgcctgcgcgggggagcccagcacagaccgccgccgggaccccgagtcgcgcaccccagccccaccgcccaccccgcgcgccatggaccccaaggaccgcaagaagatccagttctcggtgcccgcgccccctagccagctcgacccccgccaggtggagatgatccggcgcaggagaccaacgcctgccatgctgttccggctctcagagcactcctcaccagaggaggaagcctccccccaccagagagcctcaggagaggggcaccatctcaagtcgaagagacccaacccctgtgcctacacaccaccttcgctgaaagctgtgcagcgcattgctgagtctcacctgcagtctatcagcaatttgaatgagaaccaggcctcagaggaggaggatgagctgggggagcttcgggagctgggttatccaagagaggaagatgaggaggaagaggaggatgatgaagaagaggaagaagaagaggacagccaggctgaagtcctgaaggtcatcaggcagtctgctgggcaaaagacaacctgtggccagggtctggaagggccctgggagcgcccaccccctctggatgagtccgagagagatggaggctctgaggaccaagtggaagacccagcactaagtgagcctggggaggaacctcagcgcccttccccctctgagcctggcacataggcacccagcctgcatctcccaggaggaagtggaggggacatcgctgttccccagaaacccactctatcctcaccctgttttgtgctcttcccctcgcctgctagggctgcggcttctgacttctagaagactaaggctggtctgtgtttgcttgtttgcccacctttggctgatacccagagaacctgggcacttgctgcctgatgcccacccctgccagtcattcctccattcacccagcgggaggtgggatgtgagacagcccacattggaaaatccagaaaaccgggaacagggatttgcccttcacaattctactccccagatcctctcccctggacacaggagacccacagggcaggaccctaagatctggggaaaggaggtcctgagaaccttgaggtacccttagatccttttctacccactttcctatggaggattccaagtcaccacttctctcaccggcttctaccagggtccaggactaaggcgtttttctccatagcctcaacattttgggaatcttcccttaatcacccttgctcctcctgggtgcctggaagatggactggcagagacctctttgttgcgttttgtgctttgatgccaggaatgccgcctagtttatgtccccggtggggcacacagcggggggcgccaggttttccttgtcccccagctgctctgcccctttccccttcttccctgactccaggcctgaacccctcccgtgctgtaataaatctttgtaaataa

SEQ ID NO: 36 - DNA, 430 bp, Artificial Sequence
  Description of Artificial Sequence Synthetic DNA sequence
cattgacctgaaaaatccttgcttctctttttccttgagttgctctgaatgactaatattttcaggacttttccatggatcgaatgctggttgtctttttgagtggttggttccagaactgctctctgatggtagagaagaaactctagttgacacattattttcatggaaagaattgtctggacgtggagatggcactctgtaaaagctaccaactctccttggcacaggatcattgtataggtgtctattttctttgggggctgtgcaatcatcagagtgtgggtcatgatacactccaccctcagacttctgggcgtggtatgggaaggaagaggngccttccctgggagggtcnccttttgaccgaangatctttgccacagtcatcccctnggaaggggagctgtttctccaggctgggggtgac

SEQ ID NO: 37 - DNA, 520 bp, Artificial Sequence
  Description of Artificial Sequence Synthetic DNA sequence
gacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaaggnttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgattct

SEQ ID NO: 38 - DNA, 3399 bp, Artificial Sequence
  Description of Artificial Sequence Synthetic DNA sequence
cggcgacggcgtcctcaggagctgtggggtcccctgctagaagtgggggactcggcgggggagtcatttaatacttcatgattagaacaaatatgtgaaagttcccaccaaccagtgagaatttcttccttcagacggttttggatcttactgcacagctttctgagaagttcttttggtgccatgttttgtggcttgcatcaaaagaggagtttgtcttcatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtgacttctgcaagcctgcggctggtcccaatgccctgaatcacctctctcatggaagaaccaattaacaccaatgaatcaaccaaaac

SEQ ID NO: 39 - DNA, 396 bp, Artificial Sequence
  Description of Artificial Sequence Synthetic DNA sequence
natttcgccccctctctccctcctcccctcgccctcggtgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggccgcaggtctaagaccaaggaagcacgcaagcgccgtgaagagcnctccaggcaagaaggaggagatcatcaagactttatccaaggaggaagagaccaagaaataaaacctcccactttgtctgtacatactggcctctgtgattacatagatcagccattaaaataaaacaagccttaatctgcaaaa

SEQ ID NO: 40 - DNA, 698 bp, Artificial Sequence
  Description of Artificial Sequence Synthetic DNA sequence
ctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgc

SEQ ID NO: 41 - DNA, 204 bp, Artificial Sequence
  Description of Artificial Sequence Synthetic DNA sequence
ttttttttttttttttttttttttttttttttttttttcattcagatttacccaggaggttgctgtctttcanacaaagatgaggttcactggnaggaggcaaaggtgggactagggaggtgacccgcatgggccagatnggagagaaactcttcccaccccggcagaaggggcctcttcctggccgccccatccanactcagg

SEQ ID NO: 42 - DNA, 457 bp, Artificial Sequence
  Description of Artificial Sequence Synthetic DNA sequence
caggacgcctacgacatcagccagctgcgtcacccgacagcgctgagcctgcctctgggaccgccgccacttcgcagagatgccccgcagncagcctgcacccccagccaccccgagtgctgcccaccagccccctggacatcgccgacttcatcaatgatggcttggaggctgcagatagtgaccccagtgtgccgccttacgacacagccctcatctatgactacgagggtgacggctcggtggcggggacntgagctccatcctgtccagccagggcgatgaggaccaggactacgactacctcagagactgggggccccgcttcgcccggctggcagacatgtatgggcacccgtgcgggttnggagttacggggccagatgggaccaccaggccagggagggtctttctcctggggcactgctacccagacacagaggccggacagcctgan

SEQ ID NO: 43 - DNA, 2875 bp, Artificial Sequence
  Description of Artificial Sequence Synthetic DNA sequence
acttgcgctgtcactcagcctggacgcgcttcttcgggtcgcgggtgcactccggcccggctcccgcctcggccccgatggacgccgcgttcctcctcgtcctcgggctgttggcccagagcctctgcctgtctttgggggttcctggatggaggaggcccaccaccctgtacccctggcgccgggcgcctgccctgagccgcgtgcggagggcctgggtcatccccccgatcagcgtatccgagaaccacaagcgtctcccctaccccctggttcagatcaagtcggacaagcagcagctgggcagcgtcatctacagcatccagggacccggcgtggatgaggagccccggggcgtcttctctatcgacaagttcacagggaaggtcttcctcaatgccatgctggaccgcgagaagactgatcgcttcaggctaagagcgtttgccctggacctgggaggatccaccctggaggaccccacggacctggagattgtagttgtggatcagaatgacaaccggccagccttcctgcaggaggcgttcactggccgcgtgctggagggtgcagtcccaggcacctatgtgaccagggcagaggccacagatgccgacgaccccgagacggacaacgcagcgctgcggttctccatcctgcagcagggcagccccgagctcttcagcatcgacgagctcacaggagagatccgcacagtgcaagtggggctggaccgcgaggtggtcgcggtgtacaatctgaccctgcaggtggcggacatgtctggagacggcctcacagccactgcctcagccatcatcacccttgatgacatcaatgacaatgcccccgagttcaccagggatgagttcttcatggaggccatagaggccgtcagcggagtggatgtgggacgcctggaagtggaggacagggacctgccaggctccccaaactgggtggccaggttcaccatcctggaaggcgaccccgatgggcagttcaccatccgcacggaccccaagaccaacgagggtgttctgtccattgtgaaggccctggactatgagagctgtgaacactacgaactcaaagtgtcggtgcagaatgaggccccgctgcaggcggctgcccttagggctgagcggggccaggccaaggtccgcgtgcatgtgcaggacaccaacgagccccccgtgttccaggagaacccacttcggaccagcctagcagagggggcacccccaggcactctggtggccaccttctctgcccgggaccctgacacagagcagctgcagaggctcagctactccaaggactacgacccggaagactggctgcaagtggacgcagccactggccggatccagacccagcacgtgctcagcccggcgtcccccttcctcaagggcggctggtacagagccatcgtcctggcccaggatgacgcctcccagccccgcaccgccaccggcaccctgtccatcgagatcctggaggtgaacgaccatgcacctgtgctggccccgccgccgccgggcagcctgtgcagcgagccacaccaaggcccaggcctcctcctgggcgccacggatgaggacctgcccccccacggggcccccttccacttccagctgagccccaggctcccagagctcggccggaactggagcctcagccaggtcaacgtgagccacgcgcgcctgcggccgcgacaccaggtccccgaaggcctgcaccgcctcagcctgctgctccgggactcggggcagccgccccagcagcgcgagcagcctctgaacgtgaccgtgtgccgctgcggcaaggacggcgtctgcctgccgggggccgcagcgctgctggcggggggcacaggcctcagcctgggcgcactggtcatcgtgctggccagcgccctcctgctgctggtgctggtcctgctcgtggcactccgggcgcggttctggaagcagtctcggggcaaggggctgctgcacggcccccaggacgaccttcgagacaatgtcctcaactacgatgagcaaggaggcggggaggaggaccaggacgcctacgacatcagccagctgcgtcacccgacagcgctgagcctgcctctgggaccgccgccacttcgcagagatgccccgcagggccgcctgcacccccagccaccccgagtgctgcccaccagccccctggacatcgccgacttcatcaatgatggcttggaggctgcagatagtgaccccagtgtgccgccttacgacacagccctcatctatgactacgagggtgacggctcggtggcggggacgctgagctccatcctgtccagccagggcgatgaggaccaggactacgactacctcagagactgggggccccgcttcgcccggctggcagacatgtatgggcacccgtgcgggttggagtacggggccagatgggaccaccaggccagggagggtctttctcctggggcactgctacccagacacagaggccggacagcctgaccctggggcgcaactggacatgccactccccggcctcgtggcagtgatggcccctgcagaggcagcctgaggtcaccgggcccgacccccctgggcctggggcagcctccttcctgtaggcgagggcccaagtctgggggcagaacctgagtgtggatggggcggccaggaagaggccccttcctgccggggtgggaagagtttctctccatcggccccatgcgggtcacctccctagtcccacctttgcctcctaccagtgaacctcatctttgtatgaaagacagcaacctcctgggtaaatctgaatgaaaaacgtgctagtctctttcatgcaaaaaaaaaaaaaaaaa

SEQ ID NO: 44 - DNA, 438 bp, Artificial Sequence
  Description of Artificial Sequence Synthetic DNA sequence
ttttttttgtgcttcttatgtttctctgtgctgtattctggaagtggtcggataccatcgtctgagctgggactattctgataagatttctctgctatggaggagcctgactcactttcactgtccagattctggggggtatatgctggtgacttactatggctgggagagccacgctcatgctttggagtggaaccactgatgagtggagagccatagtttttagaagaagccatttgaggcctaagcccttctccactactttccccaggtttctgcaaagtcactttggctttaatgctaggggagcctccatcatgcttactgatgataatttttgccacacctgtgctccccacattttttgactctgaggtcttcttaggaagaatccactgaactcccggagtgggaaacctttngatttgtctttatcac

SEQ ID NO: 45 - DNA, 5809 bp, Artificial Sequence
  Description of Artificial Sequence Synthetic DNA sequence
gggaagatggcggcggcctcgagcacccttctcttcttgccgccggggacttcagattgatccttcccgggaagagtagggactgctggtgccctgcgtcccgggatcccgagccaacttgtttcctccgttagtggtggggaagggcttatccttttgtggcggatctagcttctcctcgccttcaggatgaaagctcagggggaaaccgaggagtcagaaaagctgagtaagatgagttctctcctggaacggctccatgcaaaatttaaccaaaatagaccctggagtgaaaccattaagcttgtgcgtcaagtcatggagaagagggttgtgatgagttctggagggcatcaacatttggtcagctgtttggagacattgcagaaggctctcaaagtaacatctttaccagcaatgactgatcgtttggagtccatagcaagacagaatggactgggctctcatctcagtgccagtggcactgaatgttacatcacgtcagatatgttctatgtggaagtgcagttagatcctgcaggacagctttgtgatgtaaaagtggctcaccatggggagaatcctgtgagctgtccggagcttgtacagcagctaagggaaaaaaattttgatgaattttctaagcaccttaagggccttgttaatctgtataaccttccaggggacaacaaactgaagactaaaatgtacttggctctccaatccttagaacaagatctttctaaaatggcaattatgtactggaaagcaactaatgctggtcccttggataagattcttcatggaagtgttggctatctcacaccaaggagtgggggtcatttaatgaacctgaagtactatgtctctccttctgacctactggatgacaagactgcatctcccatcattttgcatgagaataatgtttctcgatctttgggcatgaatgcatcagtgacaattgaaggaacatctgctgtgtacaaactcccaattgcaccattaattatggggtcacatccagttgacaataaatggaccccttccttctcctcaatcaccagtgccaacagtgttgatcttcctgcctgtttcttcttgaaatttccccagccaatcccagtatctagagcatttgttcagaaactgcagaactgcacaggaattccattgtttgaaactcaaccaacttatgcacccctgtatgaactgatcactcagtttgagctatcaaaggaccctgaccccatacctttgaatcacaacatgagattttatgctgctcttcctggtcagcagcactgctatttcctcaacaaggatgctcctcttccagatggccgaagtctacagggaacccttgttagcaaaatcacctttcagcaccctggccgagttcctcttatcctaaatctgatcagacaccaagtggcctataacaccctcattggaagctgtgtcaaaagaactattctgaaagaagattctcctgggcttctccaatttgaagtgtgtcctctctcagagtctcgtttcagcgtatcttttcagcaccctgtgaatgactccctggtgtgtgtggtaatggatgtgcaggactcaacacatgtgagctgtaaactctacaaagggctgtcggatgcactgatctgcacagatgacttcattgccaaagttgttcaaagatgtatgtccatccctgtgacgatgagggctattcggaggaaagctgaaaccattcaagccgacaccccagcactgtccctcattgcagagacagttgaagacatggtgaaaaagaacctgcccccggctagcagcccagggtatggcatgaccacaggcaacaacccaatgagtggtaccactacaccaaccaacacctttccggggggtcccattaccaccttgtttaatatgagcatgagcatcaaagatcggcatgagtcggtgggccatggggaggacttcagcaaggtgtctcagaacccaattcttaccagtttgttgcaaatcacagggaacggggggtctaccattggctcgagtccgacccctcctcatcacacgccgccacctgtctcttcgatggccggcaacaccaagaaccacccgatgctcatgaaccttcttaaagataatcctgcccaggatttctcaaccctttatggaagcagccctttagaaaggcagaactcctcttccggctcaccccgcatggaaatatgctcggggagcaacaagaccaagaaaaagaagtcatcaagattaccacctgagaaaccaaagcaccagactgaagatgactttcagagggagctattttcaatggatgttgactcacagaaccctatctttgatgtcaacatgacagctgacacgctggatacgccacacatcactccagctccaagccagtgtagcactcccccaacaacttacccacaaccagtacctcacccccaacccagtattcaaaggatggtccgactatccagttcagacagcattggcccagatgtaactgacatcctttcagacattgcagaagaagcttctaaacttcccagcactagtgatgattgcccagccattggcacccctcttcgagattcttcaagctctgggcattctcagagtaccctgtttgactctgatgtctttcaaactaacaataatgaaaatccatacactgatccagctgatcttattgcagatgctgctggaagccccagtagtgactctcctaccaatcatttttttcatgatggagtagatttcaatcctgatttattgaacagccagagccaaagtggttttggagaagaatattttgatgaaagcagccaaagtggggataatgatgatttcaaaggatttgcatctcaggcactaaatactttgggggtgccaatgcttggaggtgataatggggagaccaagtttaagggcaataaccaagccgacacagttgatttcagtattatttcagtagccggcaaagctttagctcctgcagatcttatggagcatcacagtggtagtcagggtcctttactgaccactggggacttagggaaagaaaagactcaaaagagggtaaaggaaggcaatggcaccagtaatagtactctctcggggcccggattagacagcaaaccagggaagcgcagtcggaccccttctaatgatgggaaaagcaaagataagcctccaaagcggaagaaggcagacactgagggaaagtctccatctcatagttcttctaacagaccttttaccccacctaccagtacaggtggatctaaatcgccaggcagtgcaggaagatctcagactcccccaggtgttgccacaccacccattcccaaaatcactattcagattcctaagggaacagtgatggtgggcaagccttcctctcacagtcagtataccagcagtggttctgtgtcttcctcaggcagcaaaagccaccatagccattcttcctcctcttcctcatctgcttccacctcagggaagatgaaaagcagtaaatcagaaggttcatcaagttccaagttaagtagcagtatgtattctagccaggggtcttctggatctagccagtccaaaaattcatcccagtctggggggaagccaggctcctctcccataaccaagcatggactgagcagtggctctagcagcaccaagatgaaacctcaaggaaagccatcatcacttatgaatccttctttaagtaaaccaaacatatccccttctcattcaaggccacctggaggctctgacaagcttgcctctccaatgaagcctgttcctggaactcctccatcctctaaagccaagtcccctatcagttcaggttctggtggttctcatatgtctggaactagttcaagctctggcatgaagtcatcttcagggttaggatcctcaggctcgttgtcccagaaaactcccccatcatctaattcctgtacggcatcttcctcctccttttcctcaagtggctcttccatgtcatcctctcagaaccagcatgggagttctaaaggaaaatctcccagcagaaacaagaagccgtccttgacagctgtcatagataaactgaagcatggggttgtcaccagtggccctgggggtgaagacccactggacggccagatgggggtgagcacaaattcttccagccatcctatgtcctccaaacataacatgtcaggaggagagtttcagggcaagcgtgagaaaagtgataaagacaaatcaaaggtttccacctccgggagttcagtggattcttctaagaagacctcagagtcaaaaaatgtggggagcacaggtgtggcaaaaattatcatcagtaagcatgatggaggctcccctagcattaaagccaaagtgactttgcagaaacctggggaaagtagtggagaagggcttaggcctcaaatggcttcttctaaaaactatggctctccactcatcagtggttccactccaaagcatgagcgtggctctcccagccatagtaagtcaccagcatataccccccagaatctggacagtgaaagtgagtcaggctcctccatagcagagaaatcttatcagaatagtcccagctcagacgatggtatccgaccacttccagaatacagcacagagaaacataagaagcacaaaaaggaaaagaagaaagtaaaagacaaagatagggaccgagaccgggacaaagaccgagacaagaaaaaatctcatagcatcaagccagagagttggtccaaatcacccatctcttcagaccagtccttgtctatgacaagtaacacaatcttatctgcagacagaccctcaaggctcagcccagactttatgattggggaggaagatgatgatcttatggatgtggccctgattgggaattaggaaccttatttcctaaaagaaacagggccagaggaaaaaaaactattgataagtttataggcaaaccaccataaggggtgagtcagacaggtctgatttggttaagaatcctaaatggcatggctttgacatcaagctgggtgaattagaaaggcatatccagaccctattaaagaaaccacagggtttgattctggttaccaggaagtcttctttgttcctgtgccagaaagaaagttaaaatacttgcttaagaaagggaggggggtgggaggggtgtagggagagggaagggagggaaacagttttgtgggaaatattcatatatattttcttctccctttttccatttttaggccatgttttaaactcattttagtgcatgtatatgaagggctgggcagaaaatgaaaaagcaatacattccttgatgcatttgcatgaaggttgttcaactttgtttgaggtagttgtccgtttgagtcatgggcaaatgaaggactttggtcattttggacacttaagtaatgtttggtgtctgtttcttaggagtgactgggggagggaagattattttagctatttatttgtaatattttaaccctttatctgtttgtttttatacagtgtttcgttctaaatctatgaggtttagggttcaaaatgatggaaggccgaagagcaaggcttatatggtggtagggagcttatagcttgtgctaatactgtagcatcaagcccaagcaaattagtcagagcccgcctttagagttaaatataatagaaaaaccaaaatgatatttttattttaggagggtttaaatagggttcagagatcataggaatattaggagttacctctctgtggaggtat

SEQ ID NO: 46 - DNA, 276 bp, Artificial Sequence
  Description of Artificial Sequence Synthetic DNA sequence
ttttttctttnggaaaagaatgcttctttattgatggttatatttatcagcaaggatcatgactcagtagccagttgaaggaatcagancactttgggtacatgctgctaaaagcccgtcagctcgtcatccttgtttttgtcaacctggtatccagtaagtaccaagtcctcattttgtccgggaagacttttgaatactttcaagtgcatatatttattatcacctgctcgtaccttaatcgtagtaatttgttccagcaacaacttgagtttt

SEQ ID NO: 47 - DNA, 265 bp, Artificial Sequence
  Description of Artificial Sequence Synthetic DNA sequence
aaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttcca

SEQ ID NO: 48 - DNA, 451 bp, Artificial Sequence
  Description of Artificial Sequence Synthetic DNA sequence
acttccctgttcactttggttccagcatcctgtccagcaaagaagcaatcagccaaaatgatacctggaggcttatctgaggccaaacccgccactccagaaatccaggagattgttgataaggttaaaccacagcttgaagaaaaaacaaatgagacttatggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttcca

SEQ ID NO: 49 - DNA, 410 bp, Artificial Sequence
  Description of Artificial Sequence Synthetic DNA sequence
cacgagcactgcttagaactcaagttcctgggatcaaacttgtatcacacaggctggagaaggctgtggagaaactgagtcctcccaggtctcaaggtgggtggagggagcctgcaggggtctccttccctcccctcttgcctgttctgcctggtcagagcctgcacacgagtgagagggctcccttagagagggccgggctagaggaagctgaagtttcagaataagcagcttattctgtggcctcctttccactacagactccttgaggaggagtaagaccccagaaggacaggtgagtctcacctaaggctgaccaaagtccagctcagccagcccgtgattcttatccaagacatccgccccacagcagtgaagaagcngatgccactcaaaagccattctcagtn

SEQ ID NO: 50 - DNA, 474 bp, Artificial Sequence
  Description of Artificial Sequence Synthetic DNA sequence
atttttatagataatacagatattttggtaaattgaacttggtttttctttcccagcatcgtggatgtagactgagaatggctttgagtggcatcagcttctcactgctgtgggcggatgtcttggatagatcacgggctggctgagctggactttggtcagcctaggtgagactcacctgtccttctggggtcttactcctcctcaaggagtctgtagtggaaaggaggccacagaataagctgcttattctgaaacttcagcttcctctagcccggccctctctaagggagccctctgcactcgtgtgaggctctgaccaggcagaacaggcaagaggggagggaaggagacccctgcaggctccctccanccaccttgaagacctggggaggactcagtttctcccacaagccttctccagcctgtgtgatacaagtttgatnccaggaacttgagttctaagcagtgctc

SEQ ID NO: 51 - DNA, 3737 bp, Artificial Sequence
  Description of Artificial Sequence Synthetic DNA sequence
ggcgtccgcgcacacctccccgcgccgccgccgccaccgcccgcactccgccgcctctgcccgcaaccgctgagccatccatgggggtcgcgggccgcaaccgtcccggggcggcctgggcggtgctgctgctgctgctgctgctgccgccactgctgctgctggcgggggccgtcccgccgggtcggggccgtgccgcggggccgcaggaggatgtagatgagtgtgcccaagggctagatgactgccatgccgacgccctgtgtcagaacacacccacctcctacaagtgctcctgcaagcctggctaccaaggggaaggcaggcagtgtgaggacatcgatgaatgtggaaatgagctcaatggaggctgtgtccatgactgtttgaatattccaggcaattatcgttgcacttgttttgatggcttcatgttggctcatgacggtcataattgtcttgatgtggacgagtgcctggagaacaatggcggctgccagcatacctgtgtcaacgtcatggggagctatgagtgctgctgcaaggaggggtttttcctgagtgacaatcagcacacctgcattcaccgctcggaagagggcctgagctgcatgaataaggatcacggctgtagtcacatctgcaaggaggccccaaggggcagcgtcgcctgtgagtgcaggcctggttttgagctggccaagaaccagagagactgcatcttgacctgtaaccatgggaacggtgggtgccagcactcctgtgacgatacagccgatggcccagagtgcagctgccatccacagtacaagatgcacacagatgggaggagctgccttgagcgagaggacactgtcctggaggtgacagagagcaacaccacatcagtggtggatggggataaacgggtgaaacggcggctgctcatggaaacgtgtgctgtcaacaatggaggctgtgaccgcacctgtaaggatacttcgacaggtgtccactgcagttgtcctgttggattcactctccagttggatgggaagacatgtaaagatattgatgagtgccagacccgcaatggaggttgtgatcatttctgcaaaaacatcgtgggcagttttgactgcggctgcaagaaaggatttaaattattaacagatgagaagtcttgccaagatgtggatgagtgctctttggataggacctgtgaccacagctgcatcaaccaccctggcacatttgcttgtgcttgcaaccgagggtacaccctgtatggcttcacccactgtggagacaccaatgagtgcagcatcaacaacggaggctgtcagcaggtctgtgtgaacacagtgggcagctatgaatgccagtgccaccctgggtacaagctccactggaataaaaaagactgtgtggaagtgaaggggctcctgcccacaagtgtgtcaccccgtgtgtccctgcactgcggtaagagtggtggaggagacgggtgcttcctcagatgtcactctggcattcacctctcttcagatgtcaccaccatcaggacaagtgtaacctttaagctaaatgaaggcaagtgtagtttgaaaaatgctgagctgtttcccgagggtctgcgaccagcactaccagagaagcacagctcagtaaaagagagcttccgctacgtaaaccttacatgcagctctggcaagcaagtcccaggagcccctggccgaccaagcacccctaaggaaatgtttatcactgttgagtttgagcttgaaactaaccaaaaggaggtgacagcttcttgtgacctgagctgcatcgtaaagcgaaccgagaagcggctccgtaaagccatccgcacgctcagaaaggccgtccacagggagcagtttcacctccagctctcaggcatgaacctcgacgtggctaaaaagcctcccagaacatctgaacgccaggcagagtcctgtggagtgggccagggtcatgcagaaaaccaatgtgtcagttgcagggctgggacctattatgatggagcacgagaacgctgcattttatgtccaaatggaaccttccaaaatgaggaaggacaaatgacttgtgaaccatgcccaagaccaggaaattctggggccctgaagaccccagaagcttggaatatgtctgaatgtggaggtctgtgtcaacctggtgaatattctgcagatggctttgcaccttgccagctctgtgccctgggcacgttccagcctgaagctggtcgaacttcctgcttcccctgtggaggaggccttgccaccaaacatcagggagctacttcctttcaggactgtgaaaccagagttcaatgttcacctggacatttctacaacaccaccactcaccgatgtattcgttgcccagtgggaacataccagcctgaatttggaaaaaataattgtgtttcttgcccaggaaatactacgactgactttgatggctccacaaacataacccagtgtaaaaacagaagatgtggaggggagctgggagatttcactgggtacattgaatccccaaactacccaggcaattacccagccaacaccgagtgtacgtggaccatcaacccaccccccaagcgccgcatcctgatcgtggtccctgagatcttcctgcccatagaggacgactgtggggactatctggtgatgcggaaaacctcttcatccaattctgtgacaacatatgaaacctgccagacctacgaacgccccatcgccttcacctccaggtcaaagaagctgtggattcagttcaagtccaatgaagggaacagcgctagagggttccaggtcccatacgtgacatatgatgaggactaccaggaactcattgaagacatagttcgagatggcaggctctatgcatctgagaaccatcaggaaatacttaaggataagaaacttatcaaggctctgtttgatgtcctggcccatccccagaactatttcaagtacacagcccaggagtcccgagagatgtttccaagatcgttcatccgattgctacgttccaaagtgtccaggtttttgagaccttacaaatgactcagcccacgtgccactcaatacaaatgttctgctatagggttggtgggacagagctgtcttccttctgcatgtcagcacagtcgggtattgctgcctcccgtatcagtgactcattagagttcaatttttatagataatacagatattttggtaaattgaacttggtttttctttcccagcatcgtggatgtagactgagaatggctttgagtggcatcagcttctcactgctgtgggcggatgtcttggatagatcacgggctggctgagctggactttggtcagcctaggtgagactcacctgtccttctggggtcttactcctcctcaaggagtctgtagtggaaaggaggccacagaataagctgcttattctgaaacttcagcttcctctagcccggccctctctaagggagccctctgcactcgtgtgcaggctctgaccaggcagaacaggcaagaggggagggaaggagacccctgcaggctccctccacccaccttgagacctgggaggactcagtttctccacagccttctccagcctgtgtgatacaagtttgatcccaggaacttgagttctaagcagtgctcgtgaaaaaaaaaagcagaaagaattagaaataaataaaaactaagcacttctggagacat

SEQ ID NO: 52 - DNA, 572 bp, Artificial Sequence
  Description of Artificial Sequence Synthetic DNA sequence
accagcatctcccagttcataatcacaacccttcagatttgccttattggcagctctactctggaggtttgtttagaagaagtgtgtcacccttaggccagcaccatctctttacctcctaattccacaccctcactcgctgtagacatttgctatgagctggggatgtctctcatgaccaaatgcttttcctcaaagggagagagtgctattgtagagccagaggtctggccctatgcttccggcctcctgtccctcatccatagcacctccacatacctggccctgagccttggtgtgctgtatccatccatggggctgattgtatgtaccttctacctcttggctgccttgtgaaggaattattcccatgagttggctgggaataagtgccaggatggaatgatgggtcagctgtatcagcacgtgtggcctgttcttctatgggttggacaacctcattgtaactcactctttaatctgagaggccacagcgcaattttattttatttttctcatgatgaggttttcttaacttaaaagaacatggatataaacatgctagcattata

SEQ ID NO: 53 - DNA, 3997 bp, Artificial Sequence
  Description of Artificial Sequence Synthetic DNA sequence
gcgggaggcggacgagatgcgagcgcggccgcggccccggccgctctgggcgactgtgctggcgctgggggcgctggcgggcgttggcgtaggagggcccaacatctgtaccacgcgaggtgtgagctcctgccagcagtgcctggctgtgagccccatgtgtgcctggtgctctgatgaggccctgcctctgggctcacctcgctgtgacctgaaggagaatctgctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccagatgattcgaagaatttctccatccaagtgcggcaggtggaggattaccctgtggacatctactacttgatggacctgtcttactccatgaaggatgatctgtggagcatccagaacctgggtaccaagctggccacccagatgcgaaagctcaccagtaacctgcggattggcttcggggcatttgtggacaagcctgtgtcaccatacatgtatatctccccaccagaggccctcgaaaacccctgctatgatatgaagaccacctgcttgcccatgtttggctacaaacacgtgctgacgctaactgaccaggtgacccgcttcaatgaggaagtgaagaagcagagtgtgtcacggaaccgagatgccccagagggtggctttgatgccatcatgcaggctacagtctgtgatgaaaagattggctggaggaatgatgcatcccacttgctggtgtttaccactgatgccaagactcatatagcattggacggaaggctggcaggcattgtccagcctaatgacgggcagtgtcatgttggtagtgacaatcattactctgcctccactaccatggattatccctctttggggctgatgactgagaagctatcccagaaaaacatcaatttgatctttgcagtgactgaaaatgtagtcaatctctatcagaactatagtgagctcatcccagggaccacagttggggttctgtccatggattccagcaatgtcctccagctcattgttgatgcttatgggaaaatccgttctaaagtagagctggaagtgcgtgacctccctgaagagttgtctctatccttcaatgccacctgcctcaacaatgaggtcatccctggcctcaagtcttgtatgggactcaagattggagacacggtgagcttcagcattgaggccaaggtgcgaggctgtccccaggagaaggagaagtcctttaccataaagcccgtgggcttcaaggacagcctgatcgtccaggtcacctttgattgtgactgtgcctgccaggcccaagctgaacctaatagccatcgctgcaacaatggcaatgggacctttgagtgtggggtatgccgttgtgggcctggctggctgggatcccagtgtgagtgctcagaggaggactatcgcccttcccagcaggacgaatgcagcccccgggagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccgggagccctacatgaccgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcctggtggtcctgctctcagtgatgggggccattctgctcattggccttgccgccctgctcatctggaaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaatgataagcagtcatcctcagatcattatcagcctgtgccacgattgcaggagtccctgccatcatgtttacagaggacagtatttgtggggagggatttggggctcagagtggggtaggttgggagaatgtcagtatgtggaagtgtgggtctgtgtgtgtgtatgtgggggtctgtgtgtttatgtgtgtgtgttgtgtgtgggagtgtgtaatttaaaattgtgatgtgtcctgataagctgagctccttagcctttgtcccagaatgcctcctgcagggattcttcctgcttagcttgagggtgactatggagctgagcaggtgttcttcattacctcagtgagaagccagctttcctcatcaggccattgtccctgaagagaagggcagggctgaggcctctcattccagaggaagggacaccaagccttggctctaccctgagttcataaatttatggttctcaggcctgactctcagcagctatggtaggaactgctgggcttggcagcccgggtcatctgtacctctgcctcctttcccctccctcaggccgaaggaggagtcagggagagctgaactattagagctgcctgtgccttttgccatcccctcaacccagctatggttctctcgcaagggaagtccttgcaagctaattctttgacctgttgggagtgaggatgtctgggccactcaggggtcattcatggcctgggggatgtaccagcatctcccagttcataatcacaacccttcagatttgccttattggcagctctactctggaggtttgtttagaagaagtgtgtcacccttaggccagcaccatctctttacctcctaattccacaccctcactgctgtagacatttgctatgagctggggatgtctctcatgaccaaatgcttttcctcaaagggagagagtgctattgtagagccagaggtctggccctatgcttccggcctcctgtccctcatccatagcacctccacatacctggccctgagccttggtgtgctgtatccatccatggggctgattgtatttaccttctacctcttggctgccttgtgaaggaattattcccatgagttggctgggaataagtgccaggatggaatgatgggtcagttgtatcagcacgtgtggcctgttcttctatgggttggacaacctcattttaactcagtctttaatctgagaggccacagtgcaattttattttatttttctcatgatgaggttttcttaacttaaaagaacatgtatataaacatgcttgcattatatttgtaaatttatgtgtatggcaaagaaggagagcataggaaaccacacagacttgggcagggtacagacactcccacttggcatcattcacagcaagtcactggccagtggctggatctgtgaggggctctctcatgatagaaggctatggggatagatgtgtggacacattggacctttcctgaggaagagggactgttcttttgtcccagaaaagcagtggctccattggtgttgacatacatccaacattaaaagccacccccaaatgcccaagaaaaaaagaaagacttatcaacatttgttccatgagg

SEQ ID NO: 54 - DNA, 591 bp, Artificial Sequence
  Description of Artificial Sequence Synthetic DNA sequence
cttaccaacttttaaaaacaagtaaagttttggagaatagtaccaagaactcaaatgatcctgcggtattcaaagacaaccccactgaagacgtcgaataccagtgtgttgcagataattgccattcccacgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctaacaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgt

SEQ ID NO: 55 - DNA, 3189 bp, Artificial Sequence
  Description of Artificial Sequence Synthetic DNA sequence
tttccagccatggctgccattacctgaccagcgccacagccggtctctctgcaggcgccgggagaagtgaccagagcaatttctgcttttcacagggcgggtttctcaacggtgacttgtgggcagtgccttctgctgagcgagtcatggcccgaaggcagaactaactgtgcctgcagtcttcactctcaggatgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccaggtgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccacgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctaacaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcggaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttagacagcaaggccagatgcacatccctggaaggacatccatgttccgagaagaacagatgatccctgtatttcaagacctctgtgcacttatttatgaacctgccctgctcccacagaacacagcaattcctcaggctaagctgccggttcttaaatccatcctgctaagttaatgttgggtagaaagagatacagaggggctgttgaatttcccacatacactccttccaccaagttggaacatccttggaaattggaagagcacaagaggagatccagggcaaggccattgggatattctgaaacttgaatattttgttttgtgcagagataaagaccttttccatgcaccctcatacacagaaaccaattttcttttttatactcaatcatttctagcgcatggcctggttagaggctggttttttctcttttcctttggtccttcaaaggcttgtagttttgggtagtccttgttctttggaaatacacagtgctgaccagacagcctccccctgtcccctctatgacctcgccctccacaaatgggaaaaccagactacttgggagcaccgcctgtgaaataccaacctgaagacacggttcattcaggcaacgcacaaaacagaaaatgaaggtggaacaagcacagatgttcttcaactgtttttgtctacactctttctcttttcctctaccatgctgaaggctgaaagacaggaagatggtgccatcagcaaatattattcttaattgaaaacttgaaaaaaaaaaaaaaaa

SEQ ID NO: 56 - DNA, 463 bp, Artificial Sequence
  Description of Artificial Sequence Synthetic DNA sequence
atcacaaaactaactttattatatttttcccagttcgatttttctgtcaaatatcttcactgtccctatgacttctggttcttattttcttgacacacacattgtcctcagccttctttggattttctgcacacctcttgacactccgcgttactctctgcacagattcgctctccaaagtgcttgctgcaggctggctttttgtctttcttgatctcaggcacatggagtctgaatttcctgcttctcctttccctttctgattctgcatgagaaccttcgcactcttctgccctccgctctcctctgccaccttaggctgggagctctcattctgtctagcagacctcaagcaccttttgttctcactggactttgtctctaggtatgggttttccgggctccatcatctggattctgaatggtccatctctggggaggtcttcatgggcttcttttcatt

SEQ ID NO: 57 - DNA, 478 bp, Artificial Sequence
  Description of Artificial Sequence Synthetic DNA sequence
nttcggcaccagcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcagtccctgaaaataagggaatatccctgcgctccagaccgccaaaataagactgaggcagaacagcaaataacttgaggtctttgtattagcagaaagaatagaaataaacagaatgaaaagaagcccatgaagacctccccagagatgggacattcagaatccagatgatggagcccggaaacccatacctagagacaaagtcagtgagaacaaaaggtgct

SEQ ID NO: 58 - DNA, 12515 bp, Artificial Sequence
  Description of Artificial Sequence Synthetic DNA sequence
ctaccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggaggagaaagagtggcaacctgccttcaaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgtaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggctggaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacctagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaatgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacagctgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccagtatgcactgacaagcccacgactcacgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacaccccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaacgtggacgcagaagatgtaattggcagcaggagacagccaagagcacctaaggaaaaggcccaacccctggaagacctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaagataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataacaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgttctccagggcacggtggcaggaacaactatcctcgtctgtcccaacactgagcaggcactcggtaaacacgaatgaatggataagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccagatcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgcgatctggtcccccagcctctcttgggctttcttacactaactctgtacctaccatctcctgcctcccttaggcaggcacctccaaccaccacacactccctgctgttttccctgcctggaactttcccaccagccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtgaaactaagccactaattgagtttctggctcccctcctggggttgtaagttttgttcattcatgagggccgactgtatttcctggttactgtatcccagtgaccagccacaggagatgtccaataaagtatgtgatgaaatggtcttaaaaaaaaaaaaaaaaaaaaaa

SEQ ID NO: 59 - DNA, 416 bp, Artificial Sequence
  Description of Artificial Sequence Synthetic DNA sequence
aaggccatgttttatttgctgattaatggacaaaaggcaatgtaatttattttcaagtattttcttgaaagtctgtgctcataaaaatcatgaaaagttggaaagactgttaaatcactgaaacttcaaatatatcttacacaatcttgtttgtacaaaaatacaagttaaatataaacataaagcaatcatggtaattttatgcaaatctgttttatgtgatcatcagttatatataaaagtttctcagttctgttatttgtgaaaagatcaataccagattgaatgactacctattggcaaagggccctaaaaagcttactttaagcactcatcttttacatggttaaatgcatttcctaatttgagatcacctaaacactggaaaagaaaaaaaatgaaagggcagtatgtcc

SEQ ID NO: 60 - DNA, 500 bp, Artificial Sequence
  Description of Artificial Sequence Synthetic DNA sequence
atcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatactatggacatttataatgcgtgtgagaaaatatggggtgaagatctaagacatttaatagtatcgagaagtacacaggcaccactaataatcagacctgattctggaaaccctcttgacactgtgttaaaggtttc

SEQ ID NO: 61 - DNA, 2376 bp, Artificial Sequence
  Description of Artificial Sequence Synthetic DNA sequence
cgcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattaggctttatgactgggtgtgtgttgtgtgtatgtaatacataatgtttattgtacagatgtgtggggtttgtgttttatgatacattacagccaaattatttgttggtttatggacatactgccctttcattttttttcttttccagtgtttaggtgatctcaaattaggaaatgcatttaaccatgtaaaagatgagtgctaaagtaagctttttagggccctttgccaataggtagtcattcaatctggtattgatcttttcacaaataacagaactgagaaacttttatatataactgatgatcacataaaacagatttgcataaaattaccatgattgctttatgtttatatttaacttgtatttttgtacaaacaagattgtgtaagatatatttgaagtttcagtgatttaacagtctttccaacttttcatgatttttatgagcacagactttcaagaaaatacttgaaaataaattacattgccttttgtccattaatcagcaaataaaacatggccttaacaaagttgtttgtgttattgtacaatttgaaaattatgtcgggacataccctatagaattactaaccttactgccccttgtagaatatgtattaatcattctacattaaagaaaataatggttcttactggaatgtctaggcactgtacagttattatatatcttggttgttgtattgtaccagtgaaatgccaaatttgaaaggcctgtactgcaattttatatgtcagagattgcctgtggctctaatatgcacctcaagattttaaggagataatgtttttagagagaatttctgcttccactatagaatatatacataaatgtaaaatacttacaaaagtgg

SEQ ID NO: 62 - DNA, 456 bp, Artificial Sequence
  Description of Artificial Sequence Synthetic DNA sequence
ttttttttgagaataaaattccttattttatttcaaaaaatgtaggggtggggaagtaacatgataaacattacgatcagctccctatgggttcattctgcctctgcgggggtcgggggcatacagtagctggggggcatgccattgccatggcaacccagatgcttagatgcaggtccctcctggctgcttagagctggggggactaggcgccctccccgaaagcccccattctgagttgttggtgcctgcccttcccctgaatctaagaactgattagtgggttagactgcaacagcagctcaggatcctcccagggactttccctccctcccctcttcacttggcccgtcccctcagcttaccagcacctccagcccccacctcctcctctttcttcagnttccaccctggggtccttcatgagggtaccccttccccagcccttcagggaag

SEQ ID NO: 63 - DNA, 523 bp, Artificial Sequence
  Description of Artificial Sequence Synthetic DNA sequence
ggacatcatcaggtccctgaagaagtctgggaagctgtggctggacgcctaccttcacaaatgaagccacagcccccgggacactgtggggaaggggtgcaggtggggtgatggccagaggaatgatgggcttttgttctgaggngtgtccgagaggctggtgtatgcactgctcacggaccccatgttggatctttctccctttctcctctcctttttctcttcacatctcccccatagcaccctgccctcatgggacctgccctccctcagccgtcagccatcagccatggccctcccagtgcctcctagccccttcttccaaggagcagagaggtggccaccgggggtgtnctngtcctacctccactctctgcccctaaagatgggaggagaccagcggtccatgggtctggcctgtgagtctncccttgcagctgggtcattagggatcaaccccgttttgttttttcaagatgnttttgggggttcataggggnaggtnctagttgggnaagggcct

SEQ ID NO: 64 - DNA, 3149 bp, Artificial Sequence
  Description of Artificial Sequence Synthetic DNA sequence
agggggcgcggtgggaggagtaggagaagacaaaagccgaaagcgaagagggcccgggctgcacacaccggctgggaggcagccgtctgtgcagcgagcagccggcgcggggaggccgcagtgcacggggcgtcacagtcggcaggcagcatggggaagggagggaaccagggcgagggggccgccgagcgcgaggtgtcggtgcccaccttcagctgggaggagattcagaagcataacctgcgcaccgacaggtggctggtcattgaccgcaaggtttacaacatcaccaaatggtccatccagcacccggggggccagcgggtcatcgggcactacgctggagaagatgcaacggatgccttccgcgccttccaccctgacctggaattcgtgggcaagttcttgaaacccctgctgattggtgaactggccccggaggagcccagccaggaccacggcaagaactcaaagatcactgaggacttccgggccctgaggaagacggctgaggacatgaacctgttcaagaccaaccacgtgttcttcctcctcctcctggcccacatcatcgccctggagagcattgcatggttcactgtcttttactttggcaatggctggattcctaccctcatcacggcctttgtccttgctacctctcaggcccaagctggatggctgcaacatgattatggccacctgtctgtctacagaaaacccaagtggaaccaccttgtccacaaattcgtcattggccacttaaagggtgcctctgccaactggtggaatcatcgccacttccagcaccacgccaagcctaacatcttccacaaggatcccgatgtgaacatgctgcacgtgtttgttctgggcgaatggcagcccatcgagtacggcaagaagaagctgaaatacctgccctacaatcaccagcacgaatacttcttcctgattgggccgccgctgctcatccccatgtatttccagtaccagatcatcatgaccatgatcgtccataagaactgggtggacctggcctgggccgtcagctactacatccggttcttcatcacctacatccctttctacggcatcctgggagccctccttttcctcaacttcatcaggttcctggagagccactggtttgtgtgggtcacacagatgaatcacatcgtcatggagattgaccaggaggcctaccgtgactggttcagtagccagctgacagccacctgcaacgtggagcagtccttcttcaacgactggttcagtggacaccttaacttccagattgagcaccacctcttccccaccatgccccggcacaacttacacaagatcgccccgctggtgaagtctctatgtgccaagcatggcattgaataccaggagaagccgctactgagggccctgctggacatcatcaggtccctgaagaagtctgggaagctgtggctggacgcctaccttcacaaatgaagccacagcccccgggacaccgtggggaaggggtgcaggtggggtgatggccagaggaatgatgggcttttgttctgaggggtgtccgagaggctggtgtatgcactgctcacggaccccatgttggatctttctccctttctcctctcctttttctcttcacatctcccccatagcaccctgccctcatgggacctgccctccctcagccgtcagccatcagccatggccctcccagtgcctcctagccccttcttccaaggagcagagaggtggccaccgggggtggctctgtcctacctccactctctgcccctaaagatgggaggagaccagcggtccatgggtctggcctgtgagtctccccttgcagcctggtcactaggcatcacccccgctttggttcttcagatgctcttggggttcataggggcaggtcctagtcgggcagggcccctgaccctcccggcctggcttcactctccctgacggctgccattggtccaccctttcatagagaggcctgctttgttacaaagctcgggtctccctcctgcagctcggttaagtacccgaggcctctcttaagatgtccagggccccaggcccgcgggcacagccagcccaaaccttgggccctggaagagtcctccaccccatcactagagtgctctgaccctgggctttcacgggccccattccaccgcctccccaacttgagcctgtgaccttgggaccaaagggggagtccctcgtctcttgtgactcagcagaggcagtggccacgttcagggaggggccggctggcctggaggctcagcccaccctccagcttttcctcagggtgtcctgaggtccaagattctggagcaatctgacccttctccaaaggctctgttatcagctgggcagtgccagccaatccctggccatttggccccaggggacgtgggccctgcaggctgcaggagggcactggagctgggaggtctcgtcccagccctccccatctcggggctgctgtgtggacggcgctgcctcaggcactctcctgtctgaacctgcccttactgtgtttaacctgttgctccaggatgcattctgataggagggggcggcagggctgggccttgtgacaatctgcctttcaccacatggccttgcctcggtggccctgactgtcagggagggccagggaggcagagcgggagggagtctcaggaggaggctgccctgaggggctggggagggggtacctcatgaggaccagggtggagctgagaagaggaggaggtgggggctggaggtgctggtagctgaggggacgggcaagtgagaggggagggagggaagtcctgggaggatcctgagctgctgttgcagtctaacccactaatcagttcttagattcaggggaagggcaggcaccaacaactcagaatgggggctttcggggagggcgcctagtccccccagctctaagcagccaggagggacctgcatctaagcatctgggttgccatggcaatggcatgccccccagctactgtatgcccccgacccccgcagaggcagaatgaacccatagggagctgatcgtaatgtttatcatgttacttccccacccctacattttttgaaataaaataaggaattttattctc

SEQ ID NO: 65 - DNA, 396 bp, Artificial Sequence
  Description of Artificial Sequence Synthetic DNA sequence
ttttttttttaaatatataactatatttattttgaatattaaatagtttttaaattacaagcaatttattgaatcacactatgcatcaatatacagtaaaaatcttacaatttaaaaatgtacacaatttaaactgaaagttcattgactattatattgccatgaacctcttctgcctgtgttaaagcacagcagggaggcctgggtgggtgtgacggccccctacgtcctcctctgcagacggaatccgacggtggatccatacaccagccccatgatcaagatcccgctcggcccacgtagggcctgagtgcttaaccagcacgtgtccacaccgggtcaaactgatcttcacagggctgttggggaattcaacactgttcaatggcatctttt

SEQ ID NO: 66 - DNA, 516 bp, Artificial Sequence
  Description of Artificial Sequence Synthetic DNA sequence
attgccaaaaaagcattttatacattgagttggggggtagtggatcttagtgtggtgttgcatggaggggcgagattttatatttataatcaacacgtgggttaacatgtttttttgaaatccaagcaatacacaggaaatttaagtagaataaaaattgcagcccatttttgaaatgtcagcatgtgctgtgttcagttcaggtttttgttgtttgttttgttattttttaactaataagttggttatcagtggtgggttttcaaaatgtacttgttctaataagttgtacaatgaactaaatcagtggcattctctagataatgtggggggaaggttagaatattttctggccttctatgggggtagccaccccaggaatccaatctggaattagtccctgttttgggtgggagtttgtaccattttaaaccccataaccaaaaataatcntagttttccattcccctactagcncaggtgcgganttgtcccttttgnattgaccccngtcca

SEQ ID NO: 67 - DNA, 490 bp, Artificial Sequence
  Description of Artificial Sequence Synthetic DNA sequence
tttttttttttttgacagcagagcataagtccttttaattatgtgtttgaaaaatgtcacaagtcaaaaaaggaacacaaggcaggctccggctccctccacccccgtgagagcccttgtccatttcagccttgcactcagaaagaccccgggggtcttgtagttccacgtgcttcatgtttcgtggtatctgtcagagccttaaaacaggcccacccactactgtgaaatttcaaggaaataactgattcagttaaataacagtcccaaggtagacctgggtctcacaggtgaccacccgttttaaatccagaggccttcttttctgtccaaagccactgaaatttgatctcctccttcacacattcccgggnccccaatatggccacccacctttntggacaggtgggctaacagggttttacattaatgggnaggtttggttaaaaaacatnttccaccnaacctttttgccaaagaggttagttgg

SEQ ID NO: 68 - DNA, 368 bp, Artificial Sequence
  Description of Artificial Sequence Synthetic DNA sequence
tctgcaaaagtacaagagcctctgcatgttcgaaatccccaaggagtagagtgaggctgacttccttagaaagagggggaagccaatggcctgtctccccactaccatccccaaacgctccttggggcgtggttcctgtggaccccagctcagcacgtcaagctgcaggggcggggctcctgtgctgctgcgcgcgcttcgnctgtgcgggancagcgcagagcttggctgcgcgggggttcctcgtgtagatccatatgtctagatgcataataactggagtgcctgctcgtggaagtcagaatgctcctgggaggctgcagagggngtggaggactcttccctgcctcttggggaaggggccatct

SEQ ID NO: 69 - DNA, 2222 bp, Artificial Sequence
  Description of Artificial Sequence Synthetic DNA sequence
gtagagtgcgcgacgcttttggcgacccgacctctggctaacctacccccggagccatggcctctgctggggtggcagccgggcgacaggcggaggatgtattgccgccaacgtccgaccagccgctgcctgacaccaagccgctgccgcctcctcagccgccgccggtccctgcgcctcaaccgcagcagtcgccggcgccacggcctcagtcacctgcccgcgcgagggaggaagagaactactcctttttacctttggttcacaacatcatcaaatgcatggacaaggacagcccggaggtccaccaggacctgaacgccctcaaaagcaagttccaggagatgcgcaagctcatcagcaccatgcccggcatccacctgagccccgaacagcagcagcagcagctgcagagcctccgggagcaagtcaggaccaagaatgagcttctgcaaaagtacaagagcctctgcatgttcgaaatccccaaggagtagagtgaggctgacttccttagaaagagggggaagccaatggcctgtctccccactaccatccccaaacgctccttggggcgtggttcctgtggaccccagctcagctcgtcaagctgcaggggcggggctcctgtgctgctgcgcgcgcttcgcctgtgcgggagccagcgcagagcttggctgcgccgggggttcctcgtgtagatccatatgtctagatgcataataactggagtgcctgctggtggaagtcagaatgctcctggaggctgcagagggggtggaggactctcccctgcctctggggagggggccatctgctgcgcccggccccactgacagatctgaagagcacagtaggaagggaggcggctcctctttgcttccttccctctctctcctcccacccccataggatcagtgtgtaccaggtacacattgttcctgttaacagcagcttcttgaaacatttgcatagaattcactggacgaattaagcctgcactcatatggcatagaattgtgagagaatgttttgaaaggccagagggtggcctttttccccaaacagtttggttccttttatgtttgagccagtgaagggaactacgctttgggggcttcagcctagagccctgccaggcagcccctggctccaggttccctgcctcctagcgctctcctcgccttcagctcttgctcccttcctcgttcatcaccctcagtcagtgcccaagagtggccaaaccgcttcacatctgcagtgcttccccagggttgacaaggggccgtcctttccacacaggccagaagaggtcttcaggcgaaccgaccttcccccttctggcatttcagattccccttgctctggttaaaaggtctttccctcgtggcctttgcacttgcggcagcaacgtgtactacactgcagaagggttcagtatgcaccttgtgttgagagagaggcaaccctgggggccagttcaggtggtccccaaccataagctaggtctgaaagttacacagccaagtttgagctcttaaaagttgatgaacagcctcatttccccagcttccctgatttcttccagatgggacgttttatttgtgtgctctcccttgactgtcagattgaagtaagagcagttctctccgttgcctctcgaggaggaggtgcgaagtcctggagtattgtttgggtctcggaatgggcgcataacctgcgctgaccagtttaggggcttagcagatgcctgccagctgacctcgttggcaggagggttgggtggagatgtttttagcagagcttccattagtgtagacctgtagccacctgtcagaaggtgggtggcatattggggacctgggaatgtgtgaaggaggagatcaaatttcagtggctttggacagaaaagaaggctctggatttaagcgggtggtcacctgtgagaccaggtctaccttgggactgttatttaactgaatcagttatttccttgaaatttcacagtagtgggtgggcctgttttaaggctctgacagataccacgaaacatgaagcacgtggaactacaagacccccggggtctttctgagtgcaaggctgaaatggacaagggctcctcacgggggtggagggagccggagcctgccttgtgttccttttttgacttgtgacatttttcaaacacataattaaaaggacttatgctctgctgtctcagg

SEQ ID NO: 70 - DNA, 214 bp, Artificial Sequence
  Description of Artificial Sequence Synthetic DNA sequence
tttagttgtaattctttatttgaacatcaaataggttgagaaaattgtttacaggtgctcgagcatcccgctggattctttttcaaagtgcaaaagaggtttacaagtgtgtttcattaaacaaagcaaagctgcgacaaaaccgagtcacatcagtaatagtatgcatcggcaaaagggcatattaatccatcaaacacaatttggcatttga

SEQ ID NO: 71 - DNA, 520 bp, Artificial Sequence
  Description of Artificial Sequence Synthetic DNA sequence
tgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgc

SEQ ID NO: 72 - DNA, 6450 bp, Artificial Sequence
  Description of Artificial Sequence Synthetic DNA sequence
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 73 - DNA, 205 bp, Artificial Sequence
  Description of Artificial Sequence Synthetic DNA sequence
ccccctgggtctttatttcatctttaaaaaaacaaaacaaaaaaagtaaaaactaaacagaaaagcactctgtacaaagcctggatactgacaccattgctgttccttcctcatggggggcagtactaggtttcagggacagtctctgaatgggtcgcttttgttcttagacactcccttagggccgcttcccctctcaggccag

SEQ ID NO: 74 - DNA, 238 bp, Artificial Sequence
  Description of Artificial Sequence Synthetic DNA sequence
cctcgtcgcctcgtgcgaagccttagggaagctggcctgagaggggaagcggccctaagggagtgtctaagaacaaaagcgacccattcagagaccgtccctgaaacctagtactgccccccatgaggaaggaacagcaatggtgtcagtatccaggcactgtacagagtgcttttctgtttagtttttactttttttgttttgtttttttaaagatgaaataaagacccagggggag

SEQ ID NO: 75 - DNA, 4530 bp, Artificial Sequence
  Description of Artificial Sequence Synthetic DNA sequence
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 76 - DNA, 535 bp, Artificial Sequence
  Description of Artificial Sequence Synthetic DNA sequence
ttttttttttttttttttttttttttttttatttcatctttaaaaaaacaaaacaaaaaaagtaaaaactaaacagaaaagcactctgtacaaagcctggatactgacaccattgctgttccttcctcatggggggcagtactaggtttcagggacagtctctgaatgggtcgcttttgttcttagacactcccttagggccgcttcccctctcaggccagcttccctaaggctttcagtacccaggatctggaaggaaagggccaagctgggctgtggcatccactggaccctagagtctcattgggcagggctcagaatccacaaagactccccagtgctgttcctcttccaacgaggctggaccccttccagccatctgggaactcaagcaggaaagaaggttccttaggacaggttcctggcatggcaggttcccctggaaattgtcggagggcccccccaactcttgatgccaacagaagtcaggcctttcctggtccctgaggacacataagggttcttgggatttggc

SEQ ID NO: 77 - DNA, 544 bp, Artificial Sequence
  Description of Artificial Sequence Synthetic DNA sequence
agagtacctgggtctggacgtgccagtgtgaaccagaaggccaagtccgcagaagccctgatgtgtcctcagggagccgggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtgtttttaaagatgaa

SEQ ID NO: 78 - DNA, 322 bp, Artificial Sequence
  Description of Artificial Sequence Synthetic DNA sequence
tttgaggaatacagataaatttattagttaaatactgattttccagccatttcaccttaagacaatgttaacaggtttgtgggttatggagggtatacgagggggcctttggaagaaaacaatgtaaatgatgattaaaacagaatcttggttcaaaggtattctctgctacagccagtaggattttggagtgaggggtctgggcgtgtggggaggcgtagtaatgccacagtcagctacagctctgctgagaaagaggaaggagtctccttgagctccagcatcaggggcagaaacagcaatgtgcagaggagaacgcggc

SEQ ID NO: 79 - DNA, 424 bp, Artificial Sequence
  Description of Artificial Sequence Synthetic DNA sequence
gcccgaatgacggcatctgtttgccatgtacctggatgtgacgggcccctggggacaggcccttgccccatccatccgcttgaggcatggcaccgccatgcatccctaataccaaatctgactccaaaactgtggggtgtgacacacaagtgactgaacacttcctggggagctacaggggcacttaacccaccacagcgcacctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttca

SEQ ID NO: 80 - DNA, 2226 bp, Artificial Sequence
  Description of Artificial Sequence Synthetic DNA sequence
accaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctaccaggcgggcggccccggccccttcatgcacggcgcgggcgccgcgtcctcgccagtctacctgcccacaccgcgggtgccctcctccgttctgggcctgtcctacctccagggcggaggcgcgggctctgcgtccggaggcccctcgggcggcagccccggtggggccgcgtctggtgcggggcccgggacccagcagggcagcccgggatggagccaggcgggagcgaccggagccgcttacaccccgccgccggtgtcgccgcgcttctccttcccggggaccaccgggtccctggcggcggcggcggcggctgccgccgcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgtggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtgcccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagtctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaaggatctgagaacaagcggagggccgggccctgggacccctgctccagcccgaatgacggcatctgtttgccatgtacctggatgtgacgggcccctggggacaggcccttgccccatccatccgcttgaggcatggcaccgccctgcatccctaataccaaatctgactccaaaactgtggggtgtgacacacaagtgactgagcacttcctggggagctacaggggcacttaacccaccacagcgcagcctcatcaaaatgcagctggcaacttctcccccaggtgccttccccctgctgccggcctttgctcc

SEQ ID NO: 81 - DNA, 513 bp, Artificial Sequence
  Description of Artificial Sequence Synthetic DNA sequence
gcgccgcagttactgagcgcaggaacttctcccggcgctgtctggttctccgcgcgcgaggcgagcttcgcggctctagatgtcgagtagccagcttggaaccagtgacgggcggtgggcctggggcggccagcggtgactccagatgagccggccgtccgcgttcgcccgcgcggtgcggttgtcgcggatcagcaggatcggagtgcggggctgctgggcggaggcgttggctgcaccagggacggcggcgcctgggtcccggcggcgctgaggctggtactgtgagcccaggctcagcaagctgaacacctgcccgttgttctcccattggatctgctggcgccaggccgcggagccgccgcgctcagcgcggggggctgctgttggccggcgcggtgaagggcccagtgcactagcgcgcaaactgcaaaggcccgagcaggagcacgggtcaggcgacgcattcattccttgttccagattgaccccgttcgaagaagacctggctca

SEQ ID NO: 82 - DNA, 1946 bp, Artificial Sequence
  Description of Artificial Sequence Synthetic DNA sequence
agacactgcccgctctccgggactccgcgccgctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcatcattacttgggaaatttttaatgtttattatttacatcactttgtgaattaacacagtgtttcaattctgtaattacatatttgactctttcaaagaaatccaaatttctcatgttccttttgaaattgtagtgcaaaatggtcagtattatctaaatgaatgagccaaaatgactttgaactgaaacttttctaaagtgctggaactttagtgaaacataataataatgggtttata

SEQ ID NO: 83 - DNA, 530 bp, Artificial Sequence
  Description of Artificial Sequence Synthetic DNA sequence
ttttttcagctgtgaactattggatttgagacaggaacagaacaaatcagagggccaggggagggttgtgggggagacagagtggtttaaataggggaggaggggaagttcggtgatgggggagggaggcaggtatttacaagaaggctcagggggccagagctcatcttggaatattttataacaatataaataagattctggtttgcttttccttttcgtctcgtaaaggagagagaagtgcagagttcgattctgtacaagggggcagcggcagaaggccgccgggcgggtcactgggcgtccacccggaaggacagcagcttctcggaatgcatgttgttcagggtccgcagtccggcagcttgagcagcagcaaggtgaagcgggaagtctccaagggccggttcttcagcaccagagcccgaagaagcccgcagcaggttctcctggagctgctccaccgaagcggaattttccatgcccgaagcggtctgccgagacaagcaacaccggcggtgaagaggccc

SEQ ID NO: 84 - DNA, 497 bp, Artificial Sequence
  Description of Artificial Sequence Synthetic DNA sequence
taccgaggaggagctgggcctcttcaccgcggtggtgcttgtctctgcagaccgctcgggcatggagaattccgcttcggtggagcagctccaggagacgctgctgcggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctgatccctgagccttcttgtaaatacctgcctacttgccccatcaccgaacttcccctcctcccctatttaaaccactctgtctcccccacaaccctcccctggccctctga

SEQ ID NO: 85 - DNA, 2768 bp, Artificial Sequence
  Description of Artificial Sequence Synthetic DNA sequence
ccgaggcgctccctgggatcacatggtacctgctccagtgccgcgtgcggcccgggaaccctgggctgctggcgcctgcgcagagccctctgtcccagggaaaggctcgggcaaaaggcggctgagattggcagagtgaaatattactgccgagggaacgtagcagggcacacgtctcgcctctttgcgactcggtgccccgtttctccccatcacctacttacttcctggttgcaacctctcttcctctgggacttttgcaccgggagctccagattcgctaccccgcagcgctgcggagccggcaggcagaggcaccccgtacactgcagagacccgaccctccttgctaccttctagccagaactactgcaggctgattccccctacacactctctctgctcttcccatgcaaagcagaactccgttgcctcaacgtccaacccttctgcagggctgcagtccggccaccccaagaccttgctgcagggtgcttcggatcctgatcgtgagtcgcggggtccactccccgcccttagccagtgcccagggggcaacagcggcgatcgcaacctctagtttgagtcaaggtccagtttgaatgaccgctctcagctggtgaagacatgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctggccccctgagccttcttgtaaatacctgcctccctcccccatcaccgaacttcccctcctcccctatttaaaccactctgtctcccccacaaccctcccctggccctctgatttgttctgttcctgtctcaaatccaatagttcacagctgagctggg

SEQ ID NO: 86 - DNA, 700 bp, Artificial Sequence
  Description of Artificial Sequence Synthetic DNA sequence
acgaggagcttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatatgtggatctgcatggagctcatggatacatcactagataaattctac

SEQ ID NO: 87 - DNA, 2924 bp, Artificial Sequence
  Description of Artificial Sequence Synthetic DNA sequence
ggcttctggttcggcccacctctgaaggttccagaatcgatagtgaattcgtggttccaagtttggagcttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaaaaagcagtggacttaatcggttgaccctactgtggattggtgggtttcggggtgaagcaagttcactacagcatcaatagaaagtcatctttgagataatttaaccctgcctctcagagggttttctctcccaattttctttttactccccctcttaagggggccttggaatctatagtatagaatgaactgtctagatggatgaattatgataaaggcttaggacttcaaaaggtgattaaatatttaatgatgtgtcatatgagtcctcaagcttctcagacttctcttattctttacaaaatgaatgcattggccctgacaaaaaggtgctacggtagtgatgaaattataagtagatttgtagtttgtcccatttattattttaatatttatgtttaagtgcttggttgaaaagattccattttatacaagaagggagattcaaaaaaaaaatataaggttgggttagcaatatttatagggcttttattttttaagttcaattgtgtctgtggtccagaagaaattatttaatatgcatctttgagaatattataaaaatatcaaaaaggagctcttcttgtgaaatgtctgttccagctgttgtgactgctgccatttttggaaacatctgcccaatcctgggtgatcaccacatcttttaggggaagtgacaagatgctctggtcatactctttttcccaactttggaaaacataaaaatcactcatataacagctcaaagagtaaaacatttggttcttctgacacttgtggtatagtattagtggaaagtgatttgtaatatgattttatatccacctacctattcatctacctgtgtgtatgtgtgtgtttgtgtgtctatttggcaattcacaagtcctgccaagtggtttctatgagcatctctgtttggtaaggaggacaattgtcagttttgagggggacatgtgttaaatcacagaaaaaaatggtgccttcttctgcgtttgtccctcctgccatgtgtaagttgtaaggattgcctttgtagttaatgtactctttggctttgtttgtttgttttcttcttcagtgaagcagccttactattcatagaagggctagaataggagaaaatgaaaggtagtgagtaattctttgataagatgaggaaataatgggaaaggttgaattaattcctgggcatggactaccagatgaccacaagttgcgttgaggccgcatctttcttcagcagcgtgcaatagctggctcctctataggagatgagcttcattgggagttcctagcaagttgactaaacagcaaaagttctttctcgtgggtaaatatacccacaggttctatgatttgtagctctaggtttcttgatgatcaaggagtgaagtaattgacagggaaaatatagacctatgataaataaccaggaagcattgcttttggacaaggaagaacagagggttttgattttaaaaagaagaaaaaaaaaccttattttttctttcttggcctcaagttcaatatggagaggattgcttccctgaatcctctcttccttccccttttagag

SEQ ID NO: 88 - DNA, 501 bp, Artificial Sequence
  Description of Artificial Sequence Synthetic DNA sequence
aaataaaattttgttggaatgaagcagcaaattctcacagctgttaaaagaattggactctggcatacctaactggagaataccatgctgttttttctataaaccgctggcattttatgccttttttgtttgttttgtgttgtcaactacaaagtaattctgtaaacagtacagatagccctggactgccaccacgggccaggccagggtagtcaaggttgtccatatgcacatgggcagataacctgttttcctttgacatttttttccaagtcaaattcttggaggattgtgtatttgtattataggaagtccagatcatagacttttaaaactaaaagcatcactgctgaactccagctcagtcttcccattttataatgaggactctgaagtttatagaggtcaaggacttgtccaaagctttagatatgtagtgtctgtgccttttcctcaagtttccctagagaatgtgggggctcagacagagaataaggtgca

SEQ ID NO: 89 - DNA, 648 bp, Artificial Sequence
  Description of Artificial Sequence Synthetic DNA sequence
aaactaggtaaaatttgttgttggttccttttagaccacggctgccccttccacaccccatcttgctctaatgatcaaaacatgcttgaataactgagcttagagtatacctcctatatgtccatttaagtcaggagagggggcgatatagagactaaggcacaaaattttgtttaaaactcagaatataacatgtaaaatcccatctgctagaagcccatcctgtgccagaggaaggaaaaggaggaaatttcctttctcttttaggaggcacaacagttctcttctaggatttgtttggctgactggcagtaacctagtgaatttttgaaagatgagtaatttctttggcaaccttcctcctcccttactgaaccactctcccacctcctggtggtaccattattatagaagccctctacagcctgactttctctccagcggtccaaagttatcccctcctttacccctcatccaaagttcgcactccttcaggacagctgctgtgcattagatattaggggggaaagtcatctgtntaatttacacacttgcatgaattactggatataactccttaactcagggagctatgtcatttagtgctaacaaagtagaaaaatagctcgagtgagttctaatggtgga

SEQ ID NO: 90 - DNA, 5361 bp, Artificial Sequence
  Description of Artificial Sequence Synthetic DNA sequence
ctgcaaacccagcgcaactacggtcccccggtcagacccaggatggggccagaacggacaggggccgcgccgctgccgctgctgctggtgttagcgctcagtcaaggcattttaaattgttgtttggcctacaatgttggtctcccagaagcaaaaatattttccggtccttcaagtgaacagtttgggtatgcagtgcagcagtttataaatccaaaaggcaactggttactggttggttcaccctggagtggctttcctgagaaccgaatgggagatgtgtataaatgtcctgttgacctatccactgccacatgtgaaaaactaaatttgcaaacttcaacaagcattccaaatgttactgagatgaaaaccaacatgagcctcggcttgatcctcaccaggaacatgggaactggaggttttctcacatgtggtcctctgtgggcacagcaatgtgggaatcagtattacacaacgggtgtgtgttctgacatcagtcctgattttcagctctcagccagcttctcacctgcaactcagccctgcccttccctcatagatgttgtggttgtgtgtgatgaatcaaatagtatttatccttgggatgcagtaaagaattttttggaaaaatttgtacaaggccttgatataggccccacaaagacacaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaagaaaatatgcctattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagttcttgggtacttaaacagaaacgcccttgatactaaaaatttaataaaagaaataaaagcgatcgctagtattccaacagaaagatactttttcaatgtgtctgatgaagcagctctactagaaaaggctgggacattaggagaacaaattttcagcattgaaggtactgttcaaggaggagacaactttcagatggaaatgtcacaagtgggattcagtgcagattactcttctcaaaatgatattctgatgctgggtgcagtgggagcttttggctggagtgggaccattgtccagaagacatctcatggccatttgatctttcctaaacaagcctttgaccaaattctgcaggacagaaatcacagttcatatttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccagattggctcctattttggtagtgtgctgtgttcagttgatgtggataaagacaccattacagacgtgctcttggtaggtgcaccaatgtacatgagtgacctaaagaaagaggaaggaagagtctacctgtttactatcaaaaagggcattttgggtcagcaccaatttcttgaaggccccgagggcattgaaaacactcgatttggttcagcaattgcagctctttcagacatcaacatggatggctttaatgatgtgattgttggttcaccactagaaaatcagaattctggagctgtatacatttacaatggtcatcagggcactatccgcacaaagtattcccagaaaatcttgggatccgatggagcctttaggagccatctccagtactttgggaggtccttggatggctatggagatttaaatggggattccatcaccgatgtgtctattggtgcctttggacaagtggttcaactctggtcacaaagtattgctgatgtagctatagaagcttcattcacaccagaaaaaatcactttggtcaacaagaatgctcagataattctcaaactctgcttcagtgcaaagttcagacctactaagcaaaacaatcaagtggccattgtatataacatcacacttgatgcagatggattttcatccagagtaacctccagggggttatttaaagaaaacaatgaaaggtgcctgcagaagaatatggtagtaaatcaagcacagagttgccccgagcacatcatttatatacaggagccctctgatgttgtcaactctttggatttgcgtgtggacatcagtctggaaaaccctggcactagccctgcccttgaagcctattctgagactgccaaggtcttcagtattcctttccacaaagactgtggtgaggatggactttgcatttctgatctagtcctagatgtccgacaaataccagctgctcaagaacaaccctttattgtcagcaaccaaaacaaaaggttaacattttcagtaacactgaaaaataaaagggaaagtgcatacaacactggaattgttgttgatttttcagaaaacttgttttttgcatcattctccctaccggttgatgggacagaagtaacatgccaggtggctgcatctcagaagtctgttgcctgcgatgtaggctaccctgctttaaagagagaacaacaggtgacttttactattaactttgacttcaatcttcaaaaccttcagaatcaggcgtctctcagtttccaagccttaagtgaaagccaagaagaaaacaaggctgataatttggtcaacctcaaaattcctctcctgtatgatgctgaaattcacttaacaagatctaccaacataaatttttatgaaatctcttcggatgggaatgttccttcaatcgtgcacagttttgaagatgttggtccaaaattcatcttctccctgaaggtaacaacaggaagtgttccagtaagcatggcaactgtaatcatccacatccctcagtataccaaagaaaagaacccactgatgtacctaactggggtgcaaacagacaaggctggtgacatcagttgtaatgcagatatcaatccactgaaaataggacaaacatcttcttctgtatctttcaaaagtgaaaatttcaggcacaccaaagaattgaactgcagaactgcttcctgtagtaatgttacctgctggttgaaagacgttcacatgaaaggagaatactttgttaatgtgactaccagaatttggaacgggactttcgcatcatcaacgttccagacagtacagctaacggcagctgcagaaatcaacacctataaccctgagatatatgtgattgaagataacactgttacgattcccctgatgataatgaaacctgatgagaaagccgaagtaccaacaggagttataataggaagtataattgctggaatccttttgctgttagctctggttgcaattttatggaagctcggcttcttcaaaagaaaatatgaaaagatgaccaaaaatccagatgagattgatgagaccacagagctcagtagctgaaccagcagacctacctgcagtgggaaccggcagcatcccagccagggtttgctgtttgcgtgcatggatttctttttaaatcccatattttttttatcatgtcgtaggtaaactaacctggtattttaagagaaaactgcaggtcagtttggatgaagaaattgtggggggtgggggaggtgcggggggcaggtagggaaataatagggaaaatacctattttatatgatgggggaaaaaaagtaatctttaaactggctggcccagagtttacattctaatttgcattgtgtcagaaacatgaaatgcttccaagcatgacaacttttaaagaaaaatatgatactctcagattttaagggggaaaactgttctctttaaaatatttgtctttaaacagcaactacagaagtggaagtgcttgatatgtaagtacttccacttgtgtatattttaatgaatattgatgttaacaagaggggaaaacaaaacacaggttttttcaatttatgctgctcatccaaagttgccacagatgatacttccaagtgataattttatttataaactaggtaaaatttgttgttggttccttttataccacggctgccccttccacaccccatcttgctctaatgatcaaaacatgcttgaataactgagcttagagtatacctcctatatgtccatttaagttaggagagggggcgatatagagactaaggcacaaaattttgtttaaaactcagaatataacatttatgtaaaatcccatctgctagaagcccatcctgtgccagaggaaggaaaaggaggaaatttcctttctcttttaggaggcacaacagttctcttctaggatttgtttggctgactggcagtaacctagtgaatttttgaaagatgagtaatttctttggcaaccttcctcctcccttactgaaccactctcccacctcctggtggtaccattattatagaagccctctacagcctgactttctctccagcggtccaaagttatcccctcctttacccctcatccaaagttcccactccttcaggacagctgctgtgcattagatattaggggggaaagtcatctgtttaatttacacacttgcatgaattactgtatataaactccttaacttcagggagctattttcatttagtgctaaacaagtaagaaaaataagctagagtgaatttctaaatgttggaatgttatgggatgtaaacaatgtaaagtaaaacactctcaggatttcaccagaagttacagatgaggcactggaaaccaccaccaaattagcaggtgcaccttctgtggctgtcttgtttctgaagtactttttcttccacaagagtgaatttgacctaggcaagtttgttcaaaaggtagatcctgagatgatttggtcagattgggataaggcccagcaatctgcattttaacaagcaccccagtcactaggatgcagatggaccacactttgagaaacaccacccatttctactttttgcaccttattttctctgttcctgagcccccacattctctaggagaaacttagattaaaattcacagacactacatatctaaagctttgacaagtccttgacctctataaacttcagagtcctcattataaaatgggaagactgagctggagttcagcagtgatgctttttagttttaaaagtctatgatctgatctggacttcctataatacaaatacacaatcctccaagaatttgacttggaaaag

SEQ ID NO: 91 - DNA, 416 bp, Artificial Sequence
  Description of Artificial Sequence Synthetic DNA sequence
catttataatttttagttttcttttcttttcttttcttttctttttttttttttttctgagacggagcttgctctgtcgcccaggctggagcgcagtggcaattctggactcactgcaagctctgccttccgggttcacgccattctcctgcctcagcctcccgagtagctgggactacaggcgcctgtcaccacgcccgganaagtttttttggtataatacttagttttctttgtagctaacttagtcttccaaagtccttcaagtcctctcagttgtgcttcccacccagccagtcatcagataaggctgttcttccctgttgctgctgccctgcctgctttctgggacctgcttcctgccttgggagttgggatccctccatttttgaactccaggggccttcggtttgttc

SEQ ID NO: 92 - DNA, 436 bp, Artificial Sequence
  Description of Artificial Sequence Synthetic DNA sequence
atcttgagtcgacctcctatctcattctgtaacctagaatgcctaactgtctgggaatgcagcccagtaggtttcagcctcattgtccccagcccctattcaagattcagttgctctagttcaaatgcctctgacagttgtgcttcaccaaacctagagaatgaacaaagcgaaggacctggagttcaagaatggagggatccaactccaaggcaggaagcaggtccagaaagcaggcagggcagcagcaacagggaagaacagcttatctgatgactggctgggtgggaagcacaactgagaggacttgaaggactttgggaagacttaagttagctacaaagaaaactaagtattattaccaaaaaaacttattccgggcgtggttgacagggcgcctgttagttcccagctattcggggaggctgaggcaggg

SEQ ID NO: 93 - DNA, 4122 bp, Artificial Sequence
  Description of Artificial Sequence Synthetic DNA sequence
aggaggcgacagctgccagccgaggaggcgcggcggagaggggactgcggtcagctgcgtccacttggggctgtgcggcggtcccgcgcccggcgatgttcccgggcactccctgagtagcggcagcttatcccccgcccgctagcccgccctggtccccggctcgctcgctggctggcgcggccccggccccgctctgcgtcggccccgccgcggtggaggcgcgcgagggggacgcggccggggatgagcggattgcgggtgaactcgccgccggggccccgcgaagccgtgagccgctgcttttctccgagtcgccgccctgcccttggatttgagatcatgtccatccacatcgtggcgctggggaacgagggggacacattccaccaggacaaccagccgtcggggcttatccgcacttacctggggagaagccctctggtctccggggacgagagcagcttgttgctgaacgcggccagcacggtcgcgcgtccggtgttcaccgagtatcaggccagtgcgtttgggaatgtcaagctggtggtccacgactgtcccgtctgggacatatttgacagtgattggtacacttctcgaaatctaattgggggcgctgacatcattgtgatcaaatacaacgttaatgacaagttttcattccatgaagtaaaggataattatattccagtgataaaaagagcattaaattcagttccagtaattattgctgctgttggtaccagacaaaatgaagagttaccttgtacatgcccactatgtacctcagacagagggagctgtgttagtacaactgaagggatccaacttgcaaaagaactaggagcaacctatcttgaactccacagccttgatgacttctacataggaaagtattttggaggagtgttggagtattttatgattcaagccttaaatcagaagacaagtgaaaaaatgaagaaaagaaaaatgagcaactcctttcatggaattagaccacctcaacttgaacaaccagaaaaaatgcctgtcttaaaggctgaagcgtcacattataactctgacttaaataacttgctgttctgctgccagtgtgtggacgtggtattttataaccccgatttaaagaaagttgtagaggcccacaagatcgttctctgcgctgtaagccatgttttcatgctgcttttcaatgtgaagagtcccactgacattcaggattccagtatcatccgaactacccaggatctttttgctataaacagagatactgcatttccaggtgctagccatgaatcttcaggcaacccaccattacgagtcattgttaaagacgccctcttctgttcttgtttatcagacatccttcgcttcatttattcaggtgcttttcagtgggaagaattggaagaagatatcaggaagaagttgaaagattctggggatgtttcaaatgtaatcgagaaagttaaatgcattttaaaaacaccaggaaagattaattgcctaaggaattgcaaaacctatcaagccagaaaacctttgtggttttataacacttccctcaagtttttccttaataagccgatgcttgccgatgttgtcttcgaaattcaaggtacgacagtgccagcccacagggccatcctggtggcccgttgtgaagtgatggcagccatgtttaatggtaattacatggaagcaaagagtgtcctgattcccgtttatggtgtttccaaagagactttcttgtcatttttagaatacctgtacacagactcctgctgcccagctggcatattccaggccatgtgtctcctgatctgtgccgagatgtaccaagtgtccagactgcagcacatctgtgagctgttcatcattacccagctgcagagcatgccaagcagggaactggcatccatgaaccttgatatagttgacctgcttaaaaaggccaagtttcaccactctgattgcctttcaacctggctacttcatttcattgctactaactacctcatcttcagtcaaaagcctgaatttcaggatctttcagtggaagaacgcagttttgttgaaaagcacagatggccgtcgaatatgtacttgaagcagcttgcggaatacaggaagtatattcactcccggaaatgtcgttgcttagtaatgtaacctggagcttttatacactacatttcttttttattattatgaagaatgggatacctccaggttccagtaaaattcttctgaccgaaaccaatgtgggtgttagaaaaattaccatatagcttaatatgtttattagttctctttggaaaaaaactaccactgtggtcttaaaagggaacaaaatataccataggctaaaactaaggctttcactctagaatgcaaagctgttttgcagctgttttcccttaaagatgtcctgttgctttagtgatatttagacccctctcagttaagaaatgcttagattaaaaaaaaaaaattacgtaggattaatacagaaatttaatcatgtctgattaattgctctattaaaataaggggcatttaaagacccagcataaccatttgtataatgagaaatctaggggaaaaccaatcagtccaacatgagattttaggaatagaaatttgccggccatttggaaagtgaaatgccacttagttctcaattgatgacagtgtttgaatcatcataaaaaaaatacctgcttttcatctggacaacccaattgagccactttatctccttttggcaatctgagtaggcggggaacctaggcagggctggctttcttagcgtgtaacttgtgtagcagcacagggcccacacttagaaggaccccacacttggttcaaggctctgctatagcggaaattcttaataatgtttgaagaagggccccatgatttcattttgtgctgagccctcaaaattatgtctgtttcgtggtgggaaatatcctatgttttcttgctcaaacacctttctctctgaaagcagaaaaaggcactgatataaagggaagagaaggaggctcaccggagggaagagaacatagtgaagattcccgcctttggggaggtctggaccacccagggcctccactgccaccttggctggcaagggagaaatgtgttgtgttgtcttagctttaaaacagtcacagttcttgctctatcatagatgaacaaatactttcttgatcattctgtaagaccaggaggttggtaagagtgactaaccagcctaactttaatacacatgtataaagatgttcacagagaaagatgctctgtagagaatttgctaccgaagttggctcaagaatttgtttttagtgttatttaccaagattaggacgtcagtggcttaaattctttgaattcttttcaaggactgcaagattatttgataaagagtagcatgaatcttgtgctctaatattacacagtaagttcaaagaaaggatgtaagtcaaagacttgttacatagagggaaaatggactgggatagaggacagactgatagtttctttctttcatatcacatgtatagagaaataattatatcagaaactcacaaacctagacatggaaaaacagattactgtctattgtcagcatcattttcatctgtaagtcactactggaatatatttttcttttaatttccagtgactttagaatacacacagtttttccgacttttcaaaaatttgattaaatggttttatagtataatattgggaccccataccgttagcccttgtatgtataccaacactgccaaagtaaaacattaggtcaggcatggtggctcaggcctgtaatcccagcattttgggaggctgaggcaagtggataacttgaggtcatgagttcgaaaccagcctggccaaaacagtgaaaccccgtctctactaaaaatacaaaattagccagatgtggtggcgcacacctgtaatcccagctactcaggaagctgaggcaggaaaatcgcttgaacctgggaggtggaagttgcagtgagccgagatcgcaccactgcactccagcctgggtgacaagagcgaaactccatcacaaaaaaaaaaaaaaaa

SEQ ID NO: 94 - DNA, 393 bp, Artificial Sequence
  Description of Artificial Sequence Synthetic DNA sequence
tccccaaggctgccccatcacaatgccngtgaagcttgactggcagacactgaggccngaagctgggggctgcagggggtcactggctcacccggtccccccgtaatctgtaaaacatactgggtgagggaggctnctggaggaccngaatctctcccttctccaggcagtagtgaggcatatgcctgttggccttgggccanttaaagatcattccagccccagtgctgttctctgaattcttggggaacacagggatgggggctcctaatgaggaccccagaaactctgagctctcacaactttcaaagacacttgcctncctcctttgcccanacctncaccattacagcatttgatcccanaagtaagganggggcggtnccattncac

SEQ ID NO: 95 - DNA, 2195 bp, Artificial Sequence
  Description of Artificial Sequence Synthetic DNA sequence
actctttcttcggctcgcgagctgagaggagcaggtagaggggcagaggcgggactgtcgtctgggggagccgcccaggaggctcctcaggccgaccccagaccctggctggccaggatgaagtatctccggcaccggcggcccaatgccaccctcattctggccatcggcgctttcaccctcctcctcttcagtctgctagtgtcaccacccacctgcaaggtccaggagcagccaccggcgatccccgaggccctggcctggcccactccacccacccgcccagccccggccccgtgccatgccaacacctctatggtcacccacccggacttcgccacgcagccgcagcacgttcagaacttcctcctgtacagacactgccgccactttcccctgctgcaggacgtgcccccctctaagtgcgcgcagccggtcttcctgctgctggtgatcaagtcctcccctagcaactatgtgcgccgcgagctgctgcggcgcacgtggggccgcgagcgcaaggtacggggtttgcagctgcgcctcctcttcctggtgggcacagcctccaacccgcacgaggcccgcaaggtcaaccggctgctggagctggaggcacagactcacggagacatcctgcagtgggacttccacgactccttcttcaacctcacgctcaagcaggtcctgttcttacagtggcaggagacaaggtgcgccaacgccagcttcgtgctcaacggggatgatgacgtctttgcacacacagacaacatggtcttctacctgcaggaccatgaccctggccgccacctcttcgtggggcaactgatccaaaacgtgggccccatccgggctttttggagcaagtactatgtgccagaggtggtgactcagaatgagcggtacccaccctattgtgggggtggtggcttcttgctgtcccgcttcacggccgctgccctgcgccgtgctgcccatgtcttggacatcttccccattgatgatgtcttcctgggtatgtgtctggagcttgagggactgaagcctgcctcccacagcggcatccgcacgtctggcgtgcgggctccatcgcaacacctgtcctcctttgacccctgcttctaccgagacctgctgctggtgcaccgcttcctaccttatgagatgctgctcatgtgggatgcgctgaaccagcccaacctcacctgcggcaatcagacacagatctactgagtcagcatcagggtccccagcctctgggctcctgtttccataggaaggggcgacaccttcctcccaggaagctgagacctttgtggtctgagcataagggagtgccagggaaggtttgaggtttgatgagtgaatattctggctggcgaactcctacacatccttcaaaacccacctggtactgttccagcatcttccctggatggctggaggaactccagaaaatatccatcttctttttgtggctgctaatggcagaagtgcctgtgctagagttccaactgtggatgcatccgtcccgtttgagtcaaagtcttacttccctgctctcacctactcacagacgggatgctaagcagtgcacctgcagtggtttaatggcagataagctccgtctgcagttccaggccagccagaaactcctgtgtccacatagagctgacgtgagaaatatctttcagcccaggagagaggggtcctgatcttaaccctttcctgggtctcagacaactcagaaggttggggggataccagagaggtggtggaataggaccgccccctccttacttgtgggatcaaatgctgtaatggtggaggtgtgggcagaggagggaggcaagtgtcctttgaaagttgtgagagctcagagtttctggggtcctcattaggagcccccatccctgtgttccccaagaattcagagaacagcactggggctggaatgatctttaatgggcccaaggccaacaggcatatgcctcactactgcctggagaagggagagattcaggtcctccagcagcctccctcacccagtatgttttacagattacggggggaccgggtgagccagtgaccccctgcagcccccagcttcaggcctcagtgtctgccagtcaagcttcacaggcattgtgatggggcagccttggggaatataaaattttgtgaagacttgg

SEQ ID NO: 96 - DNA, 306 bp, Artificial Sequence
  Description of Artificial Sequence Synthetic DNA sequence
attcaactcttctcggaggagcctggtgttggtgaagcagttccggccagctgtgtatgcgggtgaggtggagcgccgcttcccagggtccctagcagctgtagaccaggacgggcctcgggagctacagccagccctgcccggctcagcgggggttgacagttgagctgtgtgccggcctcgtggaccagcctgggctctcgctggaggaagtggcttgcaaggaggcttnggaggagtgtggctaccacttggccccctctgatctgcgccgggtcgccacatactngtcttgagttgggactt

SEQ ID NO: 97 - DNA, 148 bp, Artificial Sequence
  Description of Artificial Sequence Synthetic DNA sequence
ggagctatgcaagcctttattggggtccgcggggtntggggtgagtggccaagactggctctgtctagaaccctggagtctcactggagatccaggttgggggccacctggctgaggaaccatgagacaccaaagatgacgccgaggg

SEQ ID NO: 98 - DNA, 840 bp, Artificial Sequence
  Description of Artificial Sequence Synthetic DNA sequence
gtcacgcgcccgcccgaaggctcctgtcgggacagggcgccgccccgtgtcggcccccgcctgtccgggcgccgccatggagcgcatcgagggggcgtccgtgggccgctgcgccgcctcaccctacctgcggccgctcacgctgcattaccgccagaatggtgcccagaagtcctgggacttcatgaagacgcatgacagcgtgaccgttctcttattcaactcttctcggaggagcctggtgttggtgaagcagttccggccagctgtgtatgcgggtgaggtggagcgccgcttcccagggtccctagcagctgtagaccaggacgggcctcgggagctacagccagccctgcccggctcagcgggggtgacagttgagctgtgtgccggcctcgtggaccagcctgggctctcgctggaggaagtggcttgcaaggaggcttgggaggagtgtggctaccacttggccccctctgatctgcgccgggtcgccacatactggtctggagtgggactgactggctccagacagaccatgttctacacagaggtgacagatgcccagcgtagcggtccaggtgggggcctggtggaggagggtgagctcattgaggtggtgcacctgcccctggaaggcgcccaggcctttgcagacgacccggacatccccaagaccctcggcgtcatctttggtgtctcatggttcctcagccaggtggcccccaacctggatctccagtgagactccagggttctagacagaggccagtcttggccactcaccccacaccccgcggaccccaataaaggcttgcatagctccaaaaaaaaaaaaaa

SEQ ID NO: 99 - DNA, 308 bp, Artificial Sequence
  Description of Artificial Sequence Synthetic DNA sequence
ggctgcagcggggtgagcggcggcacggccggngngatcctggagccatggggctctgcgcgancgccantcctggatgcgctggagaacctgaccgccgaggagctcangaagttcaagctgaagctgctgtcggtgccgctgcgnaggggctacgggcgcatcccgcggggcgcgctgctgtccatggacgccttggacctcaccgacaagctggtnagcttctacctgganacctacggcgccaagttaaccgttaacntgttncgcaaaatgggccttaaggaattggcccggnaatttaaagg

SEQ ID NO: 100 - DNA, 459 bp, Artificial Sequence
  Description of Artificial Sequence Synthetic DNA sequence
ggctcggccgtgaacatcccgtctgatgagggccacatccccctgcacctggcggcccagcatggtcactatgatgtgtctgagatgctgctacagcaccagtctaacccgtgcatggtggacaactcggggaagacgcccctgggacctggcctgcgagttcggccgcgttggggtggtccagctgctcctcagcagcaatatgtgtgcggcgctgctgggagccccggccgggagacgccaccgaccccaacggcaccagccctttgcacctcgcagctaaaaacggccacatcgacatcatcaggtaggagccggtagcagggagggcntcagcctttaggggttccccaggggtttcagcagcagcctgggggttcaggggcaccagttttttggttttnggggataaggggcgggacaagggcagggtnccaggttccttggttcanggttttg

SEQ ID NO: 101 - DNA, 5761 bp, Artificial Sequence
  Description of Artificial Sequence Synthetic DNA sequence
ctgtgggaggcggccggtgccgcggggccgccgccgcctctgagccgcggccgagcttcacggagncgcagccgcgtcgctgcggccccggccgcgcaatggggaaggagcaggagctggtgcaggcggtgaaggcggaggacgtagggaccgcgcagaggctgctgcagaggccgcggcccgggaaggccaagctcctgggttccaccaagaagatcaatgtcaacttccaggacccggatgggttctcggctctgcaccatgcggccctgaacggcaacacggaattgatcagcctgctgctggaggcccaggccgctgtggacatcaaggacaacaaaggcatgcggccgctgcactatgcggcctggcagggccggaaggagcccatgaagctggtgctgaaggcgggctcggccgtgaacatcccgtctgatgagggccacatccccctgcacctggcggcccagcatggtcactatgatgtgtctgagatgctgctacagcaccagtctaacccgtgcatggtggacaactcggggaagacgcccctggacctggcctgcgagttcggccgcgttggggtggtccagctgctcctcagcagcaatatgtgtgcggcgctgctggagccccggccgggagacgccaccgaccccaacggcaccagccctttgcacctcgcagctaaaaacggccacatcgacatcatcaggctcctcctccaagccggcatcgacattaaccgccagaccaagtccggcacggccctgcacgaggctgcgctctgcggaaagacagaggtggtgcggctgctgctggatagcgggatcaatgcccacgtgaggaacacctacagccagacagccctggacatcgtgcaccagttcaccacgtcccaggccagcagggagatcaagcagctgttgcgagaggcctcagcggccctgcaggtccgggcgaccaaggattattgcaacaattacgacctgaccagcctcaacgtgaaggcaggggacatcatcacagtcctcgagcagcatccggatggccggtggaagggctgcatccatgacaaccggacgggcaatgaccgggtgggctacttcccgtcctccctgggcgaggccattgtcaagcgagcaggttcccgagcaggcactgaaccaagcctgccccagggaagcagctcatcgggaccctctgcacccccagaggagatctgggtgctgaggaagccttttgcaggtggggaccgaagcggcagcattagcggcatggctggcggccggggcagcgggggtcacgccctacacgcgggctctgaaggcgtcaagctcctggcaacggtgctttcccagaagtccgtctctgagtccggcccgggggacagccccgccaagcctccggaaggctctgcaggtgtggcccggtcccagcctccagtggcccacgccgggcaggtctatggggagcagccgcccaagaagctggagccagcatcggagggcaagagctctgaggccgtgagccagtggctcaccgcgttccagctgcagctctacgcccccaacttcatcagcgccggctacgacctgcccaccatcagccgcatgactcccgaggacctcacggccattggtgtcaccaagccgggccaccggaagaagatcgcggcagagatcagcggcctaagcatccctgactggctgcctgagcacaaacccgctaacctggccgtgtggctgtccatgatcggcctggcccagtactacaaggtgttggtggacaatggctacgagaacattgatttcatcaccgacatcacctgggaggacctgcaggagatcggcatcaccaagctggggcaccagaagaagctgatgctcgctgtgaggaagctggcagagctgcagaaggctgaatacgccaagtatgaggggggccccctgcgccggaaggcgccccagtctcttgaagtgatggccatcgagtcgccgcccccgcctgagcccacaccggccgactgccagtcccctaaaatgaccaccttccaggacagcgagctcagtgacgagctgcaggctgccatgactggcccggctgaggtggggcccaccactgagaagccctccagccacctgccacccaccccgagggccaccacgcggcaggactccagcctgggtggtcgggcacggcacatgagcagctcgcaggagctgctgggagatgggccccctgggcccagcagccccatgtctcgaagccaggagtacctcctggatgagggccccgcccccggcaccccgcccagggaggcccggcccggccgccacggccacagcatcaagagggccagcgtgccccccgtgcctggcaagccacggcaggtcctcccaccaggcactagccacttcacgcccccccagacgcccaccaaaacccgaccaggctctccccaggcccttgggggacctcatggtccagccccagctacggccaaggtgaagcccaccccgcagctgctgccgccgacagagcgccccatgtcaccccgctccctgcctcagtcaccgacgcaccgcggctttgcctacgtgctgccccagcccgtggagggcgaggtggggccggctgccccggggcctgcgcccccacccgtgccgacggctgtgcccacactgtgcctgccccctgaggccgacgcggagccggggcggcccaagaagcgggcccacagcctgaatcgctatgcggcgtccgacagcgagccggagcgggacgagctgctggtgcctgcggctgccggcccctatgccacggtccagcggcgcgtgggccgcagccactcagtgagggcgcccgcaggtgccgacaagaacgtcaaccgcagccagtcctttgccgtgcggccccgaaagaaggggcccccgccgcccccacccaagcgctccagctcggccctggctagtgccaacctggcggatgagccggtgcctgacgccgagcctgaggatggcctgctgggggtccgggcacagtgccggcgggccagtgacctggccggcagcgtggacacgggtagtgccggcagtgtgaagagcatcgcggccatgctggagctgtcctccattgggggtgggggccgggctgcccgcaggcctcctgagggccaccccactccccgccctgccagcccagagccgggccgggtggccaccgtgctggcctcagtgaaacacaaagaggccatcgggcctggcggggaggtggtgaaccggcgccgcacgctcagcgggccagtcaccggacttctggccactgcccgccgggggcctggggagtcggcagacccaggcccctttgtggaggatggcactggccggcagcggcctcggggtccctccaagggcgaggcgggtgtcgaaggcccgcccttggccaaggtggaagccagcgccacactcaagaggcgcatccgggccaagcagaaccagcaggagaacgtcaagttcatcctgaccgagtctgacacggtcaagcgcaggcccaaggccaaggagcgggaggccgggcctgagccaccaccgccactgtccgtgtaccataatggcactggcaccgtgcgccgccgaccggcctcggagcaggctgggcctccggagctgcctccaccgcccccgcctgccgaacccccgcccaccgacctggcgcacctacccccattgcccccgcccgagggcgaagcccggaagccggccaagccgcctgtctctcccaagcccgtcctgacgcagcctgtgcccaagctccagggctcgcccacacccacctccaagaaggtgccgctgccaggccctggcagcccagaggtgaagcgcgcccacggcacgccaccgcccgtgtctcccaagccgccgccgccgcccacagcgcccaagcccgtcaaggcggtcgcggggctgccttcgggcagcgccggcccttcacccgcaccctcgcccgcgcgacagccgcccgccgccctcgccaagccgcccggtacgccgccctcgctgggcgccagccccgccaagcccccgtcccccggcgcgcccgcgctgcacgtgcccgccaagcccccgcgagccgccgccgccgccgccgccgccgccgccgcgccccccgccccgcccgaaggcgcctcgccaggggacagcgcccggcagaaactggaggagacaagcgcgtgcctggccgcggcgctgcaggcggtggaggagaagatccggcaggaggacgcgcagggcccgcgcgactcggcggcggaaaagagcactggcagcatcctggacgacatcggcagcatgttcgacgacctggccgaccagctggatgccatgctggagtgaacgccgcctggccgggccctcccgcgccgcccgggccctccccgcacactgacctatacctcaggatgggcgcgtctgggcgcggcgcgagcggccgggcagggccctgcagaagcacaactccggcccctggacccgggccggggcgcccaccggggaccgcctggccgggggctccaagggctctaggcagaccctgcgcccgcgggtcctccccacctgctgctccccgtgcaatacctgctgggcctcctgcccgcgcgggaggggagggcgccccgggaccagggatggggcagcgcacaggcccgggcccagcacagaactgcccatcgggacgcccggccagccgctcgggcgcagcacaagggacaggggaccaaggtcagggcccccctgccccaccgcacccccagaatataagctatcaagagtattaatttattgggaatgagctgaggcggatttccccagagaaacaaaaaggtataactttaacaaatatatatttaaagaaagaaatattattgattctatagaaaaccatttaccaactgaaaggacacgaagtctagctccggacaaaagctgttagaggcccaggctgtctgcctgcggtccttcctccggccagagtggaggccccaggctgctagcacaggtggaaggctggggcccgggggcgggtgctctgccgggggctcctccgtgcttctctcctaagttctctgcgccccaagaactcgaggttgtgccttgctctcggcggccgtgtcctcctcctggcgtggctgcgtcccagggcgcgggtgggtttggtgaatgtgggtgtgtcagcgtggcagccacgtggaagcagggcatgtagcaggccaggccacgggcttgcagctcctctgagggtgtgcctggccccctctccaggggctgtgcaggcagcccatccaccccagtggagttcccacggacgcttctgctcatggtccaaggctgtgttgtgtggtcattctcgccctcccttttcctctctcctcctgccccctccaagaagtactgactggtctcctctatcgtgtttgctgatccacagtgttgggcacaactttgacgtttcttaaaaaaaaaatttaaaaaatgccgaaaaccagcgattgtggtgtggggcgggtggaggcggctggcggaggtggcgtcccggcgtgcagggccccttcctgctgtctcaccccgtgtttccctgcgagtggacgtgcccagctgtccccccaggcctctcctcagagggcggcacaggctgggctccagcaggtgggcaggggaggtctgggcaaggcgctgccgtcggctgtcaataaacagcaggaaac

SEQ ID NO: 102 - DNA, 438 bp, Artificial Sequence
  Description of Artificial Sequence Synthetic DNA sequence
caaagtttaattcaattttattttccacttttagtatttttcaaattatacaacatgcagtctgccagagtacccatacatcttcattttagaacctagaagattaccaaaattttccgtgggccagaggagggtgacttccagatcttttgttacatggactatagtacagcatcgttattgatataaaccaccattctcccctcaaaccccccggacaagtttgtccacaatttttttaatgtgaaagctactgtacagatacataaagcccagagaacacacatctgcagtacacaggacacactttacaaactagacatgtataattctacagaatgccctattccccttatatctggcacacaatgaacagtatttaaaactggatacacaatttttaaataagtaagcagacttcatggagcggcgccctcg

SEQ ID NO: 103 - DNA, 447 bp, Artificial Sequence
  Description of Artificial Sequence Synthetic DNA sequence
cgcgccgctccatgaagtctgcttacttatttaaaaattgtgtatcagttttaaatactgttcattgtgtgcagatataaggggaatagggcattctgtagaattatacatgtctagtttgtaaagtgtgtcctgtgtactgcagatgtgtgttctctgggctttatgtatctgtacagtagctttcacattaaaaaaattgtggacaaacttgtccggggggtttgaggggagaatggtggtttatatcaataacgatgctgtactatagtccatgtaacaaaagatctggaagtcaccctcctcctggcccacggaaaattttggtaatcttctaggttctaaaatgaagatgtatgggtactcctggcagactgcatgttgtaataattttgaaaaatactaaaagtggaaaattnaattgaatttaaccttnggaaaaaaaaa

SEQ ID NO: 104 - DNA, 494 bp, Artificial Sequence
  Description of Artificial Sequence Synthetic DNA sequence
acagtaagaaaagaactttattgtttattaatgtttctgtgtaaaacttaagcttttttttttttttttaaagaaacaccaccaaaaggggattagcttagtccatcccttcctcagtcatcttcccaccttcctccaaatgttatcccagaacattctggaggcagggagaaggggaggcagctaatcagagtctgagagcacgatgatctcttctggatcgcattgtgtggccacacttgtcttgcaagtaccaggccgaggaggatgtgaatggggggtttgggacagccgggctggagaagggatgcagagggagctggtcaccaggccatggctgggagagtcccaccctcgtngaaggacatcagcaactggggccaaggaagccaagggggaaggttgggccgggcagggtacatatctttttcccattcttctcatgcactgacctttgcctttccacatagctgtttccnaatggncctgatcct

SEQ ID NO: 105 - DNA, 660 bp, Artificial Sequence
  Description of Artificial Sequence Synthetic DNA sequence
ngcgaacagatgcaggagggtcaggaggatgatgaagangaggacgaagaggaagaagcagcagcaggtaaagatggagacaagagccccatgtcctcactacagatctccaatgaaaagaacctggaacctggcaaacagatcagcaaatcttcaggggagcagcaaaacaaaggacngcatagtgtcaccatcgttactgtcagaagaacccctggccccctccagcatagatgctgaaanaatcggagaacagcctgaggagctgaccctggaaggaagaaagccctgtgtctcaagctctttgagctaagagattgaagctttgcccctggataccccttcctcttgtggagacggacatttcctctnccaggaagcaatcangaggagccctttcaccactgtcttagagaatggagcaggcatggtctcttctacttccttcaatggaggcgtctctcctcacaactggggagattctggtccccctgcaaaaaatctcggaagggagaagaagcaaacaggatcagggcattaggaancagctatgttgaaaggcaaagtcagtgcatgagaagaatgggaaaaagatatgntaccttgccagccactttccccttggnttccttggcccagttgctgantcctccacgaggg

SEQ ID NO: 106 - DNA, 2549 bp, Artificial Sequence
  Description of Artificial Sequence Synthetic DNA sequence
gggaaccatgcgaggttctgagaattgcggcgagggtcgcctcgagagacggtttctgagcaggaattctgaaatccccaccacttcctccctccgggggatttgatcccctatggccaccgctaacagcatcatcgtgctggatgatgatgacgaagatgaagcagctgctcagccagggccctcccacccactccccaatgcggcctcacctggggcagaagcccctagctcctctgagcctcatggggccagaggaagcagtagttcgggcggcaagaaatgctacaagctggagaatgagaagctgttcgaagagttccttgaactttgtaagatgcagacagcagaccaccctgaggtggtcccattcctctataaccggcagcaacgtgcccactctctgtttttggcctcggcggagttctgcaacatcctctctagggtcctgtctcgggcccggagccggccagccaagctctatgtctacatcaatgagctctgcactgttctcaaggcccactcagccaaaaagaagctgaacttggcccctgccgccaccacctccaatgagccctctgggaataaccctcccacacacctctccttggaccccacaaatgctgaaaacactgcctctcagtctccaaggacccgtggttcccggcggcagatccagcgtttggagcagctgctggcgctctatgtggcagagatccggcggctgcaggaaaaggagttggatctctcagaattggatgacccagactccgcatacctgcaggaggcacggttgaagcgtaagctgatccgcctctttgggcgactatgtgagctgaaagactgctcttcactgaccggccgtgtcatagagcagcgcatcccctaccgtggcacccgctacccagaggttaacaggcgcattgagcggctcatcaacaagccagggcctgataccttccctgactatggggatgtgcttcgggctgtagagaaggcagctgcccgacacagccttggcctcccccgacagcagctccagctcatggctcaggatgccttccgagatgtgggcatcaggttacaggagcgacgtcacctcgatctcatctacaactttggctgccacctcacagatgactataggccaggcgttgaccccgcactatcagatcctgtgttggcccggcgccttcgggaaaaccggagtttggccatgagtcggctggatgaggtcatctccaaatatgcaatgttgcaagacaaaagtgaggagggcgagagaaaaaagagaagagctcggctccaaggcacctcttcccactctgcagacacccccgaagcctccttggattctggtgagggccctagtggaatggcatcccaggggtgcccttctgcctccagagctgagacagatgacgaagacgacgaggagagtgatgaggaagaggaggaggaggaggaagaagaagaggaggaggccacagattctgaagaggaggaggatctggaacagatgcaggagggtcaggaggatgatgaagaggaggacgaagaggaagaagcagcagcaggtaaagatggagacaagagccccatgtcctcactacagatctccaatgaaaagaacctggaacctggcaaacagatcagcagatcttcaggggagcagcaaaacaaaggacgcatagtgtcaccatcgttactgtcagaagaacccctggccccctccagcatagatgctgaaagcaatggagaacagcctgaggagctgaccctggaggaagaaagccctgtgtctcagctctttgagctagagattgaagctttgcccctggataccccttcctctgtggagacggacatttcctcttccaggaagcaatcagaggagcccttcaccactgtcttagagaatggagcaggcatggtctcttctacttccttcaatggaggcgtctctcctcacaactggggagattctggtcccccctgcaaaaaatctcggaaggagaagaagcaaacaggatcagggccattaggaaacagctatgtggaaaggcaaaggtcagtgcatgagaagaatgggaaaaagatatgtaccctgcccagcccaccttcccccttggcttccttggccccagttgctgattcctccacgagggtggactctcccagccatggcctggtgaccagctccctctgcatcccttctccagcccggctgtcccaaaccccccattcacagcctcctcggcctggtacttgcaagacaagtgtggccacacaatgcgatccagaagagatcatcgtgctctcagactctgattagctgcctccccttctccctgcctccagaatgttctgggataacatttggaggaaggtgggaagcagatgactgaggaagggatggactaagctaatccccttttggtggtgtttcctttaaaaaaaaaaaaaaaaaactccganaaagctttggacttcttccgccanaagttttggtcaatctcccaatcaaggttgttcggctttgtctacct

SEQ ID NO: 107 - DNA, 407 bp, Artificial Sequence
  Description of Artificial Sequence Synthetic DNA sequence
tcctggtttttttatttttatttttttaacatccttgaagaagtcataatacatttaaaacatttgtctttgtaagtaaactgaaacctcctgccacctgtgntantagggaaaactcgagctgggtaggaccagaaccatatgagtggtagaaggagaaagatgcctgggcctgggctgaacttaggttgtggtctcagacctaggatccaggggccaagtttccaagcttgcccatcaggaaggtggagcggttaagaactcatatgctaaatgccacccagtgaaggccaacagagacctcactgcccctgcttctgggcagcagctatagtgaccattggggccatcacaacgtttagacaggtttgtgcagcagcacattcctcccctgggcctttgactgg

SEQ ID NO: 108 - DNA, 2828 bp, Artificial Sequence
  Description of Artificial Sequence Synthetic DNA sequence
gcgctacggcggacccggctgggcagttccttccccagaaggagagattcctctgccatggagtcctacgatgtgatcgccaaccagcctgtcgtgatcgacaacggatccggtgtgattaaagctggttttgctggtgatcagatccccaaatactgctttccaaactatgtgggccgacccaagcacgttcgtgtcatggcaggagcccttgaaggcgacatcttcattggccccaaagctgaggagcaccgagggctgctttcaatccgctatcccatggagcatggcatcgtcaaggattggaacgacatggaacgcatttggcaatatgtctattctaaggaccagctgcagactttctcagaggagcatcctgtgctcctgactgaggcgcctttaaacccacgaaaaaaccgggaacgagctgccgaagttttcttcgagaccttcaatgtgcccgctcttttcatctccatgcaagctgtactcagcctttacgctacaggcaggaccacaggggtggtgctggattctggggatggagtcacccatgctgtgcccatctatgagggctttgccatgccccactccatcatgcgcatcgacatcgcgggccgggacgtctctcgcttcctgcgcctctacctgcgtaaggagggctacgacttccactcatcctctgagtttgagattgtcaaggccataaaagaaagagcctgttacctatccataaacccccaaaaggatgagacgctagagacagagaaagctcagtactacctgcctgatggcagcaccattgagattggtccttcccgattccgggcccctgagttgctcttcaggccagatttgattggagaggagagtgaaggcatccacgaggtcctggtgttcgccattcagaagtcagacatggacctgcggcgcacgcttttctctaacattgtcctctcaggaggctctaccctgttcaaaggttttggtgacaggctcctgagtgaagtgaagaaactagctccaaaagatgtgaagatcaggatatctgcacctcaggagagactgtattccacgtggattgggggctccatccttgcctccctggacacctttaagaagatgtgggtctccaaaaaggaatatgaggaagacggtgcccgatccatccacagaaaaaccttctaatgtcgggacatcatcttcacctctctctgaagttaactccactttaaaactcgctttcttgagtcggagtgtttgcgaggaactgcctgtgtgtgagtgcgtgtgtggatatgagtgtgtgcgcacatgcgagtgccgtgtggccctgggaccctgggcccagaaaggacgatgaactacccgcagtggtgatgcctgaggcctggggttgaccactaactggctcctgacagggaagagcgctggcagaggctgtgctccctcctcaggtggcctctggctggctgtgggggactccgtttactaccacagggagacagagggaggtaagccatcccccgggagaccttgctgctgaccatcctaggctgggctggcccaccctcacccccacccccagggtgccctgaggccccaggcagctgctgcctccactatcgatgcctcctgactgcacactgaggactgggactggggttgagttctgtctggttttgttgccattttggtttgggaggctggaaaagcaccccaagaagctattacagagactggagtcaggagagagcaggaggccctcatgttcaccagggaacaggaccacaccggccactgaaggagggcaggagcagtcctccctctgaatggctgcagagttaatgttcccagcccagtcccctttcgggggccttgggagagtttaaggcacctgctggttccaggacctcgctttccatctgttcttgttgcaatgccatcttcaaaccgttttatttattgaagtgtttgttcagttaggggctggagagagggagcttgctgcctcctgccttgctacactaatgtttacagcacctaagcttagcctccagggccccacctctcccagctgatggtgagctgacagtgtccacaggttccaggaccatttgagattggaagctacactcaaagacactcccaccaggctctttctcccttttcctcttctcactgccctggaatcaacaggctggttgctggttagattttctgaaacaggaggtaaaatttttctttggcagaggcccctaagcaagggaggggtgttggagagccagtgcccttaagactggagaaagctgcaatttaccaagttgccttttgccactgtagctgaccaggggactaggttgtagaggtgggaaggcccctctgggctgatcttgtgccattcttgaccttggacctgcttggttaaggagggagtgggccagaccagagtgccaggagctaatggagccaggcctgacacctaggagtggtccaaagccttcagcctagatggtgcaaagctggggccagcctgtcttcaccggcaccctcacctgtgacaccaagacccaccccaatccagacttcacacagtattctcccccacgccgtctatgaccaaaggcccctgccaggtgtgggtccacagcagcaggtatgtgtgaaagcaacgtagcgccccgcggactgcagtgcgcttaaccaactcacctcccttctcttagcccaagcctgtccctcgcacagcctcgcacaaaccacattgcctggtggggcccagtgtactgaaataaagtcgttccgatagacacgtcaaaaaaaaaaaa

SEQ ID NO: 109 - DNA, 528 bp, Artificial Sequence
  Description of Artificial Sequence Synthetic DNA sequence
cttngctctcccagcggcaaggagggggatgtctactctccagcacgtggcttcctctcccactcccacttcttgtgcttcctctcccctctgcccagcccctgcctcggcctcccccgtgggcctcccgccccaccccaacccccgtcacactcacacaaggttgacatcgtctgcctgtggtnccacgaacacaccaagtttcaaatcctttgttgctgccactgcctctgtgacacccccacaacaggggccagaggtggttggcacccccagtcccttgaaatcccccagaagcagctttcagagcctctccttccccctcttctacatggagggggaagaaaaaggattcaaangganttncctgaggaaatgttggatgtggccatgtttttgaatgtttttttttaaaatattttattactagcccacccatcaatttggaaagatgaaatttgctcttactcccataactgattttaangtccgaggcaaagccnagttaaaaaaggaggtaagtgtnac

SEQ ID NO: 110 - DNA, 472 bp, Artificial Sequence
  Description of Artificial Sequence Synthetic DNA sequence
aaccggaagacaccagtccgggaaggnggnanagggtgcaaatagttctacaaaccagttgnacctgagcaaggtgnacctccaagtgtgggctcattagggcaacatccattcataaaccaggaggtgggccaggtggaagtaaaatctgagaagcttgacttcaaggacagagtccagttcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactngccccagctngccacgctagctnacgaggtntctgccttcctnggcaagcagggtttntnatcaaggcccttggggggt

SEQ ID NO: 111 - DNA, 3747 bp, Artificial Sequence
  Description of Artificial Sequence Synthetic DNA sequence
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaactccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 112 - DNA, 418 bp, Artificial Sequence
  Description of Artificial Sequence Synthetic DNA sequence
ttttttttttttttgaagaggacagctaatatttattgagcactgataacacaagtatcatctgggtcaaaactccacaataattcaatgtgatactattactattcctatcttattgatacttgaagcatgaaaggcactaagttgtcagcatttacaatgatggcaagtgacagagcggcgtgaaggacggcagtgcagacctaagcctaatctgaattgccattctatgnaaatgactggtgatgttgtgtagtgtacccctgggcaanagatgggaaaaagtgantgctgggtggacatccaacaagtctgcatgacaatagcccccctgtctccagtctcctctctgatanatgacatcccccacaaaccacaaggagtggatctctctggcatganagcccaactntgtggg

SEQ ID NO: 113 - DNA, 667 bp, Artificial Sequence
  Description of Artificial Sequence Synthetic DNA sequence
gctatgacatgattacgaatttaatacgactcactatagggaatttggccctcgaggccaagaattcggcacgaggcttaccaacttttaaaaacaagtaaagttttggagaatagtaccaagaactcaaatgatcctgcggtattcaaagacaaccccactgaagacgtcgaataccagtgtgttgcagataattgccattcccacgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctaacaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgt

SEQ ID NO: 114 - DNA, 700 bp, Artificial Sequence
  Description of Artificial Sequence Synthetic DNA sequence
aaacagctatgacatgattacgaatttaatacgactcactatagggaatttggccctcgaggccaagaattcggcacgaggcctcgtgccgcctcgtgccgaagccttagggaagctggcctgagaggggaagcggccctaagggagtgtctaagaacaaaagcgacccattcagagactgtccctgaaacctagtactgccccccggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcc

SEQ ID NO: 115 - DNA, 658 bp, Artificial Sequence
  Description of Artificial Sequence Synthetic DNA sequence
cacaggaaacagctatgacatgattacgaatttaatacgactcactatagggaatttggccctcgaggccaagaattcggcacgagaccagcatctcccagttcataatcacaacccttcagatttgccttattggcagctctactctggaggtttgtttagaagaagtgtgtcacccttaggccagcaccatctctttacctcctaattccacaccctcactcgctgtagacatttgctatgagctggggatgtctctcatgaccaaatgcttttcctcaaagggagagagtgctattgtagagccagaggtctggccctatgcttccggcctcctgtccctcatccatagcacctccacatacctggccctgagccttggtgtgctgtatccatccatggggctgattgtatgtaccttctacctcttggctgccttgtgaaggaattattcccatgagttggctgggaataagtgccaggatggaatgatgggtcagctgtatcagcacgtgtggcctgttcttctatgggttggacaacctcattgtaactcactctttaatctgagaggccacagcgcaattttattttatttttctcatgatgaggttttcttaacttaaaagaacatggatataaacatgctagcattata

SEQ ID NO: 116 - DNA, 724 bp, Artificial Sequence
  Description of Artificial Sequence Synthetic DNA sequence
tgacctgattacgccaagcttggcacgaggagcttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatatgtggatctgcatggagctcatggatacatcactagataaattctac

SEQ ID NO: 117 - DNA, 1051 bp, Artificial Sequence
  Description of Artificial Sequence Synthetic DNA sequence
attancgactactangggaatttggccctcgaggccaagaattcggcacgagggcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtgactgcacaggactctgggttcctgctctgttctggggtccaaaccttggtctccctttggtcctgctgggagctccccctgcctctttcccctacttagctccttagcaaagagaccctggcctccactttgccctttgggtacaaagaaggaatagaagattccgtggccttgggggcaggagagagacactctccatgaacacttctccagccacctcatacccccttcccagggtaagtgcccacgaaagcccagtccactcttcgcctcggtaatacctgtctgatgccacagattttatttattctcccctaacccagggcaatgtcagctattggcagtaaagtggcgctacaaacactaaaaaaaaaaaaaaaaatttcntgggggccccnaaagtttattcctttttagggagggttanttttanttttggncactggnccntcttttttanaacgtcgggantgggaaaaaccctggggttacccaantanntcccccttgnaaannnnnnnnnnnnnnnnnnnnnnnnnnnnnnnannnnnaaannntttnanntntttcnaattttnnnnnnnntccntttnnggnaatttggccccnnngnnnaaaaanttnnnnnnnngnnnnnnnannnngggnnnaaant

SEQ ID NO: 118 - DNA, 781 bp, Artificial Sequence
  Description of Artificial Sequence Synthetic DNA sequence
gtcacacccggaagcaggggcccgagcggagccggccgcgatgagcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtgactgcacaggactctgggttcctgctctgttctggggtccaaaccttggtctccctttggtcctgctgggagctccccctgcctctttcccctacttagctccttagcaaagagaccctggcctccactttgccctttgggtacaaagaaggaatagaagattccgtggccttgggggcaggagagagacactctccatgaacacttctccagccacctcatacccccttcccagggtaagtgcccacgaaagcccagtccactcttcgcctcggtaatacctgtctgatgccacagattttatttattctcccctaacccagggcaatgtcagctattggcagtaaagtggcgctacaaacactaaaaaaaaaaaaaaaaaaaaaaaaaaaaa